(19) United States
(12) Patent Application Publication
(10) Pub. No.: US 2007/0037846 A1
(43) Pub. Date: Feb. 15, 2007

(54) Pyrazolidinedione derivatives

(75) Inventors: Heinz Fretz, Riehen (CH); Thomas Giller, Wintersingen (DE); Kurt Hilpert, Hofstetten (CH); Olivier Houille, Mulhouse (FR); Markus Riederer, Liestal (CH); Oliver Valdenaire, Allschwil (CH)

Correspondence Address: DICKSTEIN SHAPIRO LLP, 1177 AVENUE OF THE AMERICAS (6TH AVENUE), NEW YORK, NY, 10036-2714, US

(73) Assignee: ACTELION PHARMACEUTICALS, LTD., Allschwil (CH)

(21) Appl. No.: 10/562,623

(22) PCT Filed: Jun. 16, 2004
(86) PCT No.: PCT/EP04/06471

§ 371 Date: Jul. 5, 2006

(30) Foreign Application Priority Data
Jun. 24, 2003 (WO) PCT/EP03/06616

(51) Int. Cl. A61K 31/4152; A61K 31/4709; A61K 31/416

(52) U.S. Cl. 514/310; 514/404

(57) ABSTRACT

The present invention relates to compounds of alkylidene pyrazolidinedione derivatives, which are effective platelet ADP receptor antagonists that prevent platelet aggregation and thrombosis. Thus, the present invention also relates to pharmaceutical compositions that contain the compounds as well as methods of preventing or treating peripheral, visceral, hepatic, renal, cardio- and cerebro-vascular diseases and conditions that are associated with platelet aggregation, including thrombosis, in humans and other mammals. The present invention further provides a process for manufacturing the alkylidene pyrazolidinedione derivatives.

The present invention relates to alkylidene pyrazolidinedione derivatives which are effective platelet ADP receptor antagonists and can be used for the prevention and/or treatment of peripheral vascular, of visceral-, hepatic- and renal-vascular, of cardiovascular and of cerebrovascular diseases or conditions associated with platelet aggregation, including thrombosis in humans and other mammals.

Hemostasis is referred to as the cooperation of complex, interrelated events maintaining the fluidity of the blood in the vascular system while allowing the rapid formation of a solid blood clot to prevent excessive blood loss (hemorrhage) subsequent to blood vessel injury. Immediately after vascular damage, a cascade of processes is initiated, such as contraction of the vessels, platelet adhesion and aggregation, activation of the coagulation cascade and later also of the fibrinolytic system. Hemostasis is initiated by adhesion of blood platelets or thrombocytes to the exposed, highly thrombogenic, subendothelial connective tissue of the damaged vessels and aggregate to form a platelet plug to stop bleeding.

Pathological malfunction of hemostasis can result in developing of an unwanted, in some instances life-threatening, intravascular thrombus. Conditions such as turbulent blood flow in diseased vessels, disruption of underlying vessel wall, for example most commonly due to arteriosclerosis, exposure of damaged endothelial cells and release of mediators from circulating cells, activate platelet adhesion and aggregation resulting in arterial thrombus formation and hence block off arterial blood vessels causing serious disease. Thrombi also form as a consequence of stasis or slow blood flow in veins. Venous thrombi can easily embolize, that means portions of them detach and travel through the circulatory system eventually blocking other vessels. Venous thrombosis, thrombophlebitis, arterial embolism, coronary and cerebral arterial thrombosis, unstable angina, myocardial infarction, stroke, cerebral embolisms, kidney and pulmonary embolisms are serious conditions that are the common cause of death and disability in patients with vascular disease.

Initial stimuli, such as thrombin, collagen, von Willebrand factor (vWf), thromboxane A2 (TxA2) and ADP, activate platelets by binding to their respective cell surface receptors. Many of these receptors belong to the family of seven transmembrane helices containing G-protein-coupled receptors, and their importance in platelet activation has been demonstrated (Offermans S. et al., Nature 1998, 389 (11), 183-185). Upon activation, platelets change shape from a disc shape to a round form with pseudopodia, which enforces subsequent platelet adhesion and aggregation. The final common event of aggregation culminates in cross-linking the platelets by binding of fibrinogen to its receptor, glycoprotein IIb-IIIa (GPIIb-IIIa, integrin α_{IIb}β₃) receptor.

A series of antiplatelet agents have been developed over the past several years (see review, Dogne et al., Curr. Med. Chem. 2002, 9(5), 577-589). One of the first and so far most widely used agents in antiplatelet therapy is aspirin, which irreversibly inhibits the enzyme cyclooxygenase-1 and thereby affecting the TxA2 pathway. Although not optimally efficacious, treatment with aspirin remains the standard therapy against which new therapeutics are compared and judged. Following aspirin, the phosphodiesterase inhibitors dipyridamole and cilostazol have been introduced as antiplatelet agents. Antiplatelet efficacy was also obtained with antibodies against the GPIIb/IIIa receptor (The EPIC investigators, New Engl. J. Med. 1994, 330, 956-961). Currently, three intravenously applicable, potent GPIIb/IIIa receptor antagonists (abciximab, eptifibatide, and tirofiban) blocking platelet aggregation are available on the market. In addition, orally active GPIIb/IIIa antagonists like sibrafiban, xemilofiban, and orbofiban were under clinical evaluation but have not been successful so far. Indirect or direct thrombin inhibitors, e.g. unfractionated heparin, low molecular weight heparins, hirudin, have also been shown to act as highly effective antithrombotic agents (Wong G. C. et al., JAMA 2003 January 15, 289(3), 331-42; Antman E. M., Circulation 1994, 90, 1624-1630, (GUSTO) IIa Investigators, Circulation 1994, 90, 1631-1637, Neuhaus K. L. et al., Circulation 1994, 90, 1638-1642).

Adenosine 5′-diphosphate (ADP) was identified as a key mediator in platelet activation and aggregation acting on at least two platelet ADP receptors of the G-protein coupled P2 receptor family (Shaver S. R., Curr. Opin. Drug Dicovery & Development 2001, 4 (5), 665-670). The P2Y₁ receptor initiates aggregation through mobilization of calcium stores and is required for platelet shape change. The more recently identified P2Y₁₂ receptor, also denoted P2Y_{ADP}, P2Y_{AC}, P2Y_{cyc}, P_{2T}, P2T_{AC}, (see review, Barnard E. A. and Simon J., Trends Pharmacol. Sci. 2001, 22 (8), 388-391), mediates inhibition of adenylyl cyclase and is essential for full aggregation response to ADP and the stabilization of aggregates (Gachet Ch., Thromb Hemost. 2001, 86, 222-32; Turner N. A. et al., Blood 2001, 98 (12), 3340-3345; Remijin J. A. et al., Arterioscler. Thromb. Vasc. Biol. 2002, 22, 686-691).

A variety of antagonists of the platelet ADP receptor displaying inhibition of platelet aggregation and antithrombotic activity have been reported. So far, the most effective antagonists known are the thienopyridines ticlopidine, clopidogrel and CS-747, which have been used clinically as antithrombotic agents (Kam and Nethery, Anaesthesia 2003, 58, 28-35; CAPRIE Steering Committee, The Lancet 1996, 348, 1329-39; Doggrell S. A., Drugs of the Future 2001, 26 (9), 835-840). It has been demonstrated that these drugs irreversibly block the adenosine 5′-diphosphate (ADP) receptor subtype P2Y₁₂ via their reactive metabolites.

Some analogues of the endogenous antagonist ATP, for example AR-C (formerly FPL or ARL) 67085MX and AR-C69931MX (Cangrelor), reached phase II clinical studies. These inhibitors are selective platelet ADP receptor antagonists, which inhibit ADP-dependent platelet aggregation, and are effective in vivo (see review, Chattaraj S. C., Curr. Opin. Invest. Drugs, 2001, 2(2), 250-255).

Laibelman A. M. et al. (PCT application WO 99/36425, published Jul. 22, 1999) disclose fused heterotricyclic compounds, which are effective platelet ADP receptor inhibitors.

Hardern D. et al. (PCT application WO 01/36438, published May 25, 2001) disclose a series of triazolo[4,5-d]pyrimidines active as ADP receptor antagonists.

Scarborough and Marlowe (PCT application WO 01/85722, published Nov. 15, 2001) disclose tricyclic benzothiazolo[2,3-c]thiadiazine derivatives, which are effective inhibitors of the platelet ADP receptor (P2Y₁₂).

Boyer et al. (PCT application WO 02/16381, published Feb. 28, 2002, and US 2002/0052377, published May 2, 2002) disclose mononucleoside and dinucleoside polyphosphates as P2Y₁₂ receptor antagonists.

Bryant J. et al. (PCT application WO 02/098856, published Dec. 12, 2002) disclose quinoline derivatives, useful as antithrombotic agents via inhibition of the platelet ADP receptor.

Scarborough R. M. et al. (PCT application WO 03/011872, published Feb. 13, 2003) disclose sulfonylurea and sulfonamide derivatives, which are effective platelet ADP receptor inhibitors.

Alkylidene 3,5-pyrazolidinediones, the class to which the compounds of the present invention belong, have been known for a long time (Michaelis A. and Burmeister R. Ber. 1892, 1502-1513). However, the derivatives described herein display hitherto unknown biological effects, and parts of them are novel.

Bombrun A. et al. (PCT application WO 02/102359, published Dec. 27, 2002) disclose the use of alkylidene pyrazolidinedione derivatives for the treatment and/or prevention of diabetes type I and/or II, impaired glucose tolerance, insulin resistance, hyperglycemia, obesity, and polycystic ovary syndrome via inhibition of phosphotyrosine phosphatases (PTPs), in particular PTPLB, TC-PTP, SHP and GLEPP-1.

Hassan S. (Canadian patent application CA 2,012,634, published Sep. 20, 1991) claims alkylidene pyrazolidinedione derivatives blocking platelet activating factor (PAF) and leukotriene D4 (LTD4).

Krogdal T. G. (PCT application WO 00/54771, published Sep. 21, 2000) discloses 3,5-pyrazolidinedione derivatives to combat viral infections.

In conclusion, moderate oral efficacy and adverse effects like serious bleeding problems limit the use of the currently known anti-platelet and anticoagulant agents. There remains a pronounced medical need for more effective, orally active therapeutic modalities that can be used in the prevention and/or treatment of vascular diseases, particularly those related to thrombosis, with minimal side effects. In particular, there is a need for potent, selective, and orally active platelet ADP receptor (P2Y₁₂) receptor antagonists. The present invention provides compounds with such valuable pharmacological properties.

In one aspect the present invention relates to the use of pyrazolidinedione derivatives of the general formula 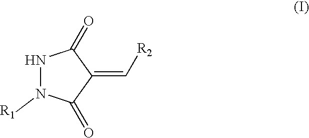
wherein
R₁ is hydrogen, optionally substituted alkyl, cycloalkyl, aryl, arylalkyl, heteroaryl, heteroarylalkyl or alkanoyl; and
R₂ is aryl or heteroaryl;
tautomers thereof;
geometric isomers thereof and tautomers of these geometric isomers, including mixtures of individual compounds of formula (I), or tautomers thereof, and their geometric isomers, or tautomers thereof;
pharmaceutically acceptable acid addition salts of compounds which are basic;
pharmaceutically acceptable salts of compounds containing acidic groups with bases;
pharmaceutically acceptable esters of compounds containing hydroxy or carboxy groups;
prodrugs of compounds in which a prodrug forming group is present; as well as hydrates or solvates thereof;
as platelet adenosine diphosphate receptor antagonists for the prevention and/or treatment of peripheral vascular, of visceral-, hepatic- and renal-vascular, of cardiovascular and of cerebrovascular diseases or conditions associated with platelet aggregation, including thrombosis, and, respectively, for the manufacture of corresponding medicaments.

The aforementioned geometric isomers of the compounds of formula (I) have the following formula (II) 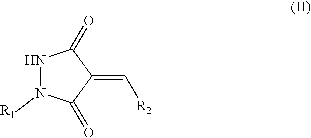
and the aforementioned tautomers of the two geometric isomers of formulae (I) and (II) have the following formulae (IA) and (IIA), respectively.

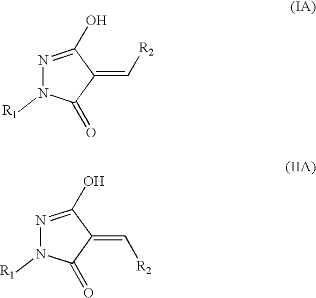

In the compounds of formula (I) R₁ is preferably hydrogen, alkyl, aryl, heteroaryl or alkanoyl, particularly hydrogen, alkyl, phenyl, bromophenyl, chlorophenyl, fluorophenyl, methylphenyl, methoxyphenyl, cyanophenyl, alkoxycarbonylphenyl, pyridinyl or alkanoyl, more particularly hydrogen, methyl, phenyl, 2-pyridinyl, 4-pyridinyl, 2-methylphenyl, 4-methylphenyl, 4-methoxyphenyl, 2-chlorophenyl, 4-fluorophenyl, 4-bromophenyl, 4-cyanophenyl, 4-ethoxycarbonylphenyl or acetyl.

R₂ is preferably naphthalenyl, thienyl or pyridyl, particularly naphthalen-2-yl, pyridin-3-yl or thiophen-3-yl.

Particularly preferred is the use of compounds of the general formula 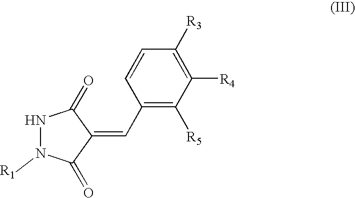
including their geometric isomers and tautomers and mixtures thereof as well as their salts, esters and prodrugs mentioned hereinabove, wherein
R₁ is as defined hereinabove;
R₃ is hydrogen, alkyl, alkenyl, alkynyl, alkoxy, hydroxyalkoxy, alkoxyalkoxy, alkenyloxy, cycloalkoxy, cycloalkylalkoxy or alkylsulfonyloxy;
R₄ is hydrogen, halogen, hydroxy, alkyl or alkoxy; and
R₅ is hydrogen, halogen, hydroxy, alkyl, alkoxy, alkoxyalkoxy, hydroxyalkoxy, dihydroxyalkoxy, alkanoyloxyalkoxy, carboxyalkoxy, carboxy-hydroxyalkoxy, carboxy-dihydroxyalkoxy, alkoxycarbonylalkoxy, alkoxycarbonyl-hydroxyalkoxy, alkoxycarbonyl-dihydroxyalkoxy, carbamoylalkoxy, N-alkylcarbamoylalkoxy, N,N-dialkyaminolalkoxy, morpholin-4-ylalkoxy, piperidin-1-ylalkoxy, morpholin-4-ylcarbonylalkoxy, 2,2-dialkyl[1,3]dioxolan-4-ylalkoxy or 2,2-dialkyl-4-carboxy[1,3]dioxolan-5-ylalkoxy; or
R₄ und R₅, together with the phenyl ring to which they are attached, form a fused, optionally substituted carbocyclic or heterocyclic ring system.

In one aspect R₃ in formula (III) may be alkyl, alkenyl, alkynyl, alkoxy, cycloalkoxy, cycloalkylalkoxy, hydroxyalkoxy or alkoxyalkoxy and R₄ and R₅ both may be hydrogen, or R₄ may be halogen, alkyl or alkoxy and R₅ may be hydrogen, or R₄ and R₅ each independently may be alkyl or alkoxy. In this aspect R₃ is preferably methyl, ethyl, propyl, iso-propyl, butyl, iso-butyl, tert-butyl, pentyl, hexyl, but-1-enyl, pent-1-enyl, but-1-ynyl, pent-1-ynyl, methoxy, ethoxy, propoxy, butoxy, iso-butoxy, 3-methyl-butoxy, pentyloxy, cyclopentyloxy, hexyloxy, cyclopropylmethoxy, cyclobutylmethoxy, 2-hydroxy-ethoxy, 2-methoxy-ethoxy, and preferably R₄ and R₅ both are hydrogen or R₄ is chloro, bromo, methyl or methoxy and R₅ is hydrogen, or R₄ and R₅ each independently are methyl or methoxy.

In a further aspect R₃ in formula (III) may be hydrogen or alkoxy and R₄ and R₅ together with the phenyl ring to which they are attached, may form an optionally substituted naphthalene, tetrahydronaphthalene, indane, 1H-indene, isoquinoline, dihydro-benzo[1,4]dioxine or benzo[1,3]dioxole moiety. In this aspect R₃ is preferably propoxy and R₄ and R₅ together with the phenyl ring to which they are attached, preferably form a naphthalene-1-yl, indan-4-yl, isoquinolin-5-yl, isoquinolin-8-yl, 1,2,3,4-tetrahydroisoquinolin-8-yl, 2-alkoxycarbonyl-1,2,3,4-tetrahydroisoquinolin-8-yl or 5,6,7,8-tetrahydronaphthalen-1-yl moiety.

In a preferred aspect R₃, R₄ and R₅ in formula (III) each are hydrogen; or

R₃ and R₄ each are hydrogen and R₄ is methoxy; or

R₃ and R₄ each are hydrogen and R₅ is methoxy; or

R₄ and R₅ each are hydrogen and R₃ is tert-butyl, ethoxy, propoxy or butoxy; or

R₃ is hydrogen, R₄ is methoxy, and R₅ is hydroxy; or

R₄ is hydrogen, R₃ is methoxy or propoxy and R₅ is methoxy or propoxy; or

R₅ is hydrogen, R₃ is methoxy, ethoxy, propoxy, butyloxy, iso-butyloxy, pentyloxy, hexyloxy, 3-methylbutoxy, 2-hydroxyethoxy, 2-methoxyethoxy, cyclopropylmethoxy or cyclobutylmethoxy and R₄ is methyl, methoxy, chloro or bromo; or

R₃ is methoxy, propoxy, cyclopentyloxy, pent-1-ynyl or ethanesulfonyloxy;

R₄ is methyl;

R₅ is hydroxy, methyl, pentyl, methoxy, propoxy, 2-methoxyethoxy, 2-hydroxyethoxy, 3-hydroxypropoxy, 4-hydroxybutoxy, 2,3-dihydroxypropoxy, 4-acetoxybutoxy, carboxymethoxy, 3-carboxypropoxy, 4-carboxybutoxy, 3-carboxy-2-hydroxypropoxy, 3-carboxy-2,3-dihydroxypropoxy, ethoxycarbonylmethoxy, 3-ethoxycarbonylpropoxy, 4-ethoxycarbonylbutoxy, 3-ethoxycarbonyl-2-hydroxypropoxy, 3-ethoxycarbonyl-2,3-dihydroxypropoxy, carbamoylmethoxy 3-N-ethylcarbamoylpropoxy, 4-N-ethylcarbamoylbutoxy, 2-N,N-dimethylaminoethoxy, 3-N,N-dmethylaminopropoxy, 2-(morpholin-4-yl)-ethyoxy, 2-(piperidin-1-yl)-ethoxy, 3-(morpholin-4-yl)-carbonylpropoxy, 4-(morpholin-4-yl)-carbonylbutoxy, 2,2-dimethyl[1,3]dioxolan-4-ylmethoxy or 2,2-dimethyl-4-carboxy[1,3]dioxolan-5-ylmethoxy; or

R₄ and R₅, together with the phenyl ring to which they are attached, form a naphthalen-1-yl, 5,6,7,8-tetrahydronaphthalen-1-yl, indan-4-yl, 1,2,3,4,-tetraisoquinolin-8-yl or 2-tert-butoxycarbonyl-1,2,3,4,-tetraisoquinolin-8-yl moiety.

Particularly preferred is the use of
4-(2,3-dimethyl-4-propoxy-benzylidene)-1-phenyl-pyrazolidine-3,5-dione;
4-(4-methoxy-3-methyl-benzylidene)-1-phenyl-pyrazolidine-3,5-dione;
4-(4-ethoxy-benzylidene)-1-phenyl-pyrazolidine-3,5-dione;
4-(4-ethoxy-3-methoxy-benzylidene)-1-phenyl-pyrazolidine-3,5-dione;
4-(2,4-dimethoxy-benzylidene)-1-phenyl-pyrazolidine-3,5-dione;
4-naphthalen-2-ylmethylene-1-phenyl-pyrazolidine-3,5-dione;
4-(4-tert-butyl-benzylidene)-1-phenyl-pyrazolidine-3,5-dione;
4-(2,3-dimethyl-4-methoxybenzylidene)-1-phenyl-pyrazolidine-3,5-dione;
4-(2,4-dimethoxy-3-methyl-benzylidene)-1-phenyl-pyrazolidine-3,5-dione;
4-(3-bromo-4-methoxy-benzylidene)-1-phenyl-pyrazolidine-3,5-dione;
1-phenyl-4-(4-propoxy-benzylidene)-pyrazolidine-3,5-dione;
4-(4-butoxy-benzylidene)-1-phenyl-pyrazolidine-3,5-dione;
4-(4-ethoxy-3-methyl-benzylidene)-1-phenyl-pyrazolidine-3,5-dione;
4-(3-methyl-4-propoxy-benzylidene)-1-phenyl-pyrazolidine-3,5-dione;
4-(4-butoxy-3-methyl-benzylidene)-1-phenyl-pyrazolidine-3,5-dione;
4-(4-hexyloxy-3-methyl-benzylidene)-1-phenyl-pyrazolidine-3,5-dione;
4-(3-methyl-4-pentyloxy-benzylidene)-1-phenyl-pyrazolidine-3,5-dione;
4-(4-cyclobutylmethoxy-3-methyl-benzylidene)-1-phenyl-pyrazolidine-3,5-dione;
4-[3-methyl-4-(3-methyl-butoxy)-benzylidene]-1-phenyl-pyrazolidine-3,5-dione;
4-(4-iso-butoxy-3-methyl-benzylidene)-1-phenyl-pyrazolidine-3,5-dione;
4-[4-(2-methoxy-ethoxy)-3-methyl-benzylidene]-1-phenyl-pyrazolidine-3,5-dione;
4-(3-chloro-4-propoxy-benzylidene)-1-phenyl-pyrazolidine-3,5-dione;
4-[4-(2-hydroxy-ethoxy)-3-methyl-benzylidene]-1-phenyl-pyrazolidine-3,5-dione;
4-(4-cyclopropylmethoxy-3-methyl-benzylidene)-1-phenyl-pyrazolidine-3,5-dione;
4-(4-cyclopentyloxy-2,3-dimethyl-benzylidene)-1-phenyl-pyrazolidine-3,5-dione;
1-phenyl-4-(4-propoxy-5,6,7,8-tetrahydro-naphthalen-1-ylmethylene)-pyrazolidine-3,5-dione;
4-(2,3-dimethyl-4-pent-1-ynyl-benzylidene)-1-phenyl-pyrazolidine-3,5-dione;
4-(2,3-dimethyl-4-pentyl-benzylidene)-1-phenyl-pyrazolidine-3,5-dione;
1-phenyl-4-(4-propoxy-naphthalen-1-ylmethylene)-pyrazolidine-3,5-dione;
1-phenyl-4-[1-(7-propoxy-indan-4-yl)-methylidene]-pyrazolidine-3,5-dione;
1-phenyl-4-[1-(5-propoxy-isoquinolin-8-yl)-methylidene]-pyrazolidine-3,5-dione;
1-phenyl-4-[1-(8-propoxy-isoquinolin-5-yl)-methylidene]-pyrazolidine-3,5-dione;
4-[1-(2-tert-butoxycarbonyl-5-propoxy-1,2,3,4-tetrahydro-isoquinolin-8-yl)-methylidene]-1-phenyl-pyrazolidine-3,5-dione;
4-(2,3-dimethyl-4-ethanesulfonyloxy-benzylidene)-1-phenyl-pyrazolidine-3,5-dione;
4-[1-(2,4-dipropoxy-phenyl)-methylidene]-1-phenyl-pyrazolidine-3,5-dione;
4-[1-(2,6-dipropoxy-pyridin-3-yl)-methylidene]-1-phenyl-pyrazolidine-3,5-dione;
4-[1-(2-hydroxy-3-methyl-4-propoxy-phenyl)-methylidene]-1-phenyl-pyrazolidine-3,5-dione;
4-[1-(2-methoxy-3-methyl-4-propoxy-phenyl)-methylidene]-1-phenyl-pyrazolidine-3,5-dione;
4-[1-(3-methyl-2,4-dipropoxy-phenyl)-methylidene]-1-phenyl-pyrazolidine-3,5-dione;
4-[1-[2-(2-methoxy-ethoxy)-3-methyl-4-propoxy-phenyl]-methylidene]-1-phenyl-pyrazolidine-3,5-dione;
4-[1-[2-(2-hydroxy-ethoxy)-3-methyl-4-propoxy-phenyl]-methylidene]-1-phenyl-pyrazolidine-3,5-dione;
4-[1-[2-(3-hydroxy-propoxy)-3-methyl-4-propoxy-phenyl]-methylidene]-1-phenyl-pyrazolidine-3,5-dione;
4-[1-[2-(4-acetoxy-butoxy)-3-methyl-4-propoxy-phenyl]-methylidene]-1-phenyl-pyrazolidine-3,5-dione;
4-[1-[2-(4-hydroxy-butoxy)-3-methyl-4-propoxy-phenyl]-methylidene]-1-phenyl-pyrazolidine-3,5-dione;
4-[1-[2-(ethoxycarbonyl-methoxy)-3-methyl-4-propoxy-phenyl]-methylidene]-1-phenyl-pyrazolidine-3,5-dione;
4-[1-[2-(carboxy-methoxy)-3-methyl-4-propoxy-phenyl]-methylidene]-1-phenyl-pyrazolidine-3,5-dione;
4-[1-[2-(2-amino-2-oxo-ethyloxy)-3-methyl-4-propoxy-phenyl]-methylidene]-1-phenyl-pyrazolidine-3,5-dione;
4-[1-[2-(3-ethoxycarbonyl-propoxy)-3-methyl-4-propoxy-phenyl]-methylidene]-1-phenyl-pyrazolidine-3,5-dione;
4-[1-[2-(3-carboxy-propoxy)-3-methyl-4-propoxy-phenyl]-methylidene]-1-phenyl-pyrazolidine-3,5-dione;
4-[1-[2-(4-ethylamino-4-oxo-butoxy)-3-methyl-4-propoxy-phenyl]-methylidene]-1-phenyl-pyrazolidine-3,5-dione;
4-[1-[3-methyl-2-(4-morpholin-4-yl-4-oxo-butoxy)-4-propoxy-phenyl]-methylidene]-1-phenyl-pyrazolidine-3,5-dione;
4-[1-[2-(4-ethoxycarbonyl-butoxy)-3-methyl-4-propoxy-phenyl]-methylidene]-1-phenyl-pyrazolidine-3,5-dione;
4-[1-[2-(4-carboxy-butyloxy)-3-methyl-4-propoxy-phenyl]-methylidene]-1-phenyl-pyrazolidine-3,5-dione;
4-[1-[2-(5-ethylamino-5-oxo-pentyloxy)-3-methyl-4-propoxy-phenyl]-methylidene]-1-phenyl-pyrazolidine-3,5-dione;
4-[1-[3-methyl-2-(5-morpholin-4-yl-s-oxo-pentyloxy)-4-propoxy-phenyl]-methylidene]-1-phenyl-pyrazolidine-3,5-dione;
4-[1-[2-(2-dimethylamino-ethoxy)-3-methyl-4-propoxy-phenyl]-methylidene]-1-phenyl-pyrazolidine-3,5-dione and its hydrochloride salt;
4-[1-[3-methyl-2-(2-morpholin-4-yl-ethoxy)-4-propoxy-phenyl]-methylidene]-1-phenyl-pyrazolidine-3,5-dione and its hydrochloride salt;
4-[1-[3-methyl-2-(2-piperidin-1-yl-ethoxy)-4-propoxy-phenyl]-methylidene]-1-phenyl-pyrazolidine-3,5-dione and its hydrochloride salt;
4-[1-[2-(3-dimethylamino-propoxy)-3-methyl-4-propoxy-phenyl]-methylidene]-1-phenyl-pyrazolidine-3,5-dione and its hydrochloride salt;
4-[1-[2-(2,2-dimethyl-[1,3]dioxolan-4-ylmethoxy)-3-methyl-4-propoxy-phenyl]-methylidene]-1-phenyl-pyrazolidine-3,5-dione;
4-[1-[2-((4R)-2,2-dimethyl-[1,3]dioxolan-4-ylmethoxy)-3-methyl-4-propoxy-phenyl]-methylidene]-1-phenyl-pyrazolidine-3,5-dione;
4-[1-[2-((4S)-2,2-dimethyl-[1,3]dioxolan-4-ylmethoxy)-3-methyl-4-propoxy-phenyl]-methylidene]-1-phenyl-pyrazolidine-3,5-dione;
4-[1-[2-((2R)-3-ethoxycarbonyl-2-hydroxy-propoxy)-3-methyl-4-propoxy-phenyl]-methylidene]-1-phenyl-pyrazolidine-3,5-dione;
4-[1-[2-((2R)-3-carboxy-2-hydroxy-propoxy)-3-methyl-4-propoxy-phenyl]-methylidene]-1-phenyl-pyrazolidine-3,5-dione;
4-[1-[2-((2S)-3-carboxy-2-hydroxy-propoxy)-3-methyl-4-propoxy-phenyl]-methylidene]-1-phenyl-pyrazolidine-3,5-dione;
4-[1-[2-((4R,5S)-4-carboxy-2,2-dimethyl-[1,3]dioxolan-5-ylmethoxy)-3-methyl-4-propoxy-phenyl]-methylidene]-1-phenyl-pyrazolidine-3,5-dione;
1-phenyl-4-[1-(5-propoxy-1,2,3,4-tetrahydro-isoquinolin-8-yl)-methylidene]-pyrazolidine-3,5-dione and its formate salt;
4-[1-[2-(2,3-dihydroxy-propoxy)-3-methyl-4-propoxy-phenyl]-methylidene]-1-phenyl-pyrazolidine-3,5-dione;
4-[1-[2-((2S)-2,3-dihydroxy-propoxy)-3-methyl-4-propoxy-phenyl]-methylidene]-1-phenyl-pyrazolidine-3,5-dione;
4-[1-[2-((2R)-2,3-dihydroxy-propoxy)-3-methyl-4-propoxy-phenyl]-methylidene]-1-phenyl-pyrazolidine-3,5-dione;
4-[1-[2-((2S,3R)-2,3-dihydroxy-3-ethoxycarbonyl-propoxy)-3-methyl-4-propoxy-phenyl]-methylidene]-1-phenyl-pyrazolidine-3,5-dione;
4-[1-[2-((2S,3R)-3-carboxy-2,3-dihydroxy-propoxy)-3-methyl-4-propoxy-phenyl]-methylidene]-1-phenyl-pyrazolidine-3,5-dione;
ethyl 4-(4-benzylidene-3,5-dioxo-pyrazolidin-1-yl)-benzoate;
ethyl 4-[4-(2-hydroxy-3-methoxy-benzylidene)-3,5-dioxo-pyrazolidin-1-yl]-benzoate;
ethyl 4-[4-(2-methoxy-benzylidene)-3,5-dioxo-pyrazolidin-1-yl]-benzoate;
ethyl 4-[4-(3-methoxy-benzylidene)-3,5-dioxo-pyrazolidin-1-yl]-benzoate;
ethyl 4-(3,5-dioxo-4-pyridin-3-ylmethylene-pyrazolidin-1-yl)-benzoate;
ethyl 4-(3,5-dioxo-4-thiophen-3-ylmethylene-pyrazolidin-1-yl)-benzoate;
ethyl 4-[4-(2,3-dimethyl-4-propoxy-benzylidene)-3,5-dioxo-pyrazolidin-1-yl]-benzoate;
4-(3-methyl-4-propoxy-benzylidene)-1-pyridin-2-yl-pyrazolidine-3,5-dione;
4-(2,3-dimethyl-4-propoxy-benzylidene)-1-pyridin-2-yl-pyrazolidine-3,5-dione;
1-(4-bromo-phenyl)-4-(2,3-dimethyl-4-propoxy-benzylidene)-pyrazolidine-3,5-dione;
4-(2,3-dimethyl-4-propoxy-benzylidene)-1-(4-methoxy-phenyl)-pyrazolidine-3,5-dione;
4-[4-(2,3-dimethyl-4-propoxy-benzylidene)-3,5-dioxo-pyrazolidin-1-yl]-benzonitrile;
4-(2,3-dimethyl-4-propoxy-benzylidene)-1-(4-fluoro-phenyl)-pyrazolidine-3,5-dione;
4-(2,3-dimethyl-4-propoxy-benzylidene)-1-(4-methyl-phenyl)-pyrazolidine-3,5-dione;
1-(2-chloro-phenyl)-4-(2,3-dimethyl-4-propoxy-benzylidene)-pyrazolidine-3,5-dione;
4-(2,3-dimethyl-4-propoxy-benzylidene)-1-(2-methyl-phenyl)-pyrazolidine-3,5-dione;
4-(2,3-dimethyl-4-propoxy-benzylidene)-pyrazolidine-3,5-dione;
4-(4-cyclopentyloxy-2,3-dimethyl-benzylidene)-pyrazolidine-3,5-dione;
4-(4-propoxy-5,6,7,8-tetrahydro-naphthalen-1-ylmethylene)-pyrazolidine-3,5-dione;
4-(2,3-dimethyl-4-pent-1-ynyl-benzylidene)-pyrazolidine-3,5-dione;
4-(2,3-dimethyl-4-pentyl-benzylidene)-pyrazolidine-3,5-dione;
4-(4-propoxy-naphthalen-1-ylmethylene)-pyrazolidine-3,5-dione;
4-(7-propoxy-indan-4-ylmethylene)-pyrazolidine-3,5-dione;
4-(2-methoxy-3-methyl-4-propoxy-benzylidene)-pyrazolidine-3,5-dione;
4-(3-methyl-2,4-dipropoxy-benzylidene)-pyrazolidine-3,5-dione;
4-(2,3-dimethyl-4-propoxy-benzylidene)-1-methyl-pyrazolidine-3,5-dione;
4-(2,3-dimethyl-4-propoxy-benzylidene)-1-pyridin-4-yl-pyrazolidine-3,5-dione; and
1-acetyl-4-[1-(2,3-dimethyl-4-propoxy-phenyl)-methylidene]-pyrazolidine-3,5-dione.

Compounds of the above general formula (III) wherein R₃, R₄ and R₅ are other than hydrogen are novel, with the exception of 1-phenyl-4-(2,3,4-trimethoxy-benzylidene)-pyrazolidine-3,5-dione. In a particular aspect the present invention thus relates to these novel compounds per se as well as for use as pharmaceutically active ingredients; to pharmaceutical compositions containing one or several of these novel compounds; to the use of these novel compounds as platelet adenosine diphosphate receptor antagonists for the prevention and/or treatment of peripheral vascular, of visceral-, hepatic- and renal-vascular, of cardiovascular and of cerebrovascular diseases and conditions associated with platelet aggregation, including thrombosis, and, respectively, for the manufacture of corresponding medicaments; and to the manufacture of these novel compounds.

The novel compounds provided by the present invention are compounds of the general formula 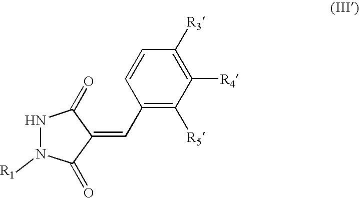
including their geometric isomers and tautomers and mixtures thereof as well as their salts, esters and prodrugs mentioned hereinabove, wherein
R₁ is as defined hereinabove;
R₃′ is alkyl, alkenyl, alkynyl, alkoxy, hydroxyalkoxy, alkoxyalkoxy, alkenyloxy, cycloalkoxy, cycloalkylalkoxy or alkylsulfonyloxy;
R₄′ is halogen, hydroxy, alkyl or alkoxy; and
R₅′ is halogen, hydroxy, alkyl, alkoxy, alkoxyalkoxy, hydroxyalkoxy, dihydroxyalkoxy, alkanoyloxyalkoxy, carboxyalkoxy, carboxy-hydroxyalkoxy, carboxy-dihydroxyalkoxy, alkoxycarbonylalkoxy, alkoxycarbonyl-hydroxyalkoxy, alkoxycarbonyl-dihydroxyalkoxy, carbamoylalkoxy, N-alkylcarbamoylalkoxy, N,N-dialkyaminolalkoxy, morpholin-4-ylalkoxy, piperidin-1-ylalkoxy, morpholin-4-ylcarbonylalkoxy, 2,2-dialkyl[1,3]dioxolan-4-ylalkoxy or 2,2-dialkyl-4-carboxy[1,3]dioxolan-5-ylalkoxy; or
R₄′ and R₅′, together with the phenyl ring to which they are attached, form a fused, optionally substituted carbocyclic or heterocyclic ring system;
with the proviso that if R₁ is phenyl and R₃′ is methoxy, R₄′ and R₅′ may not both be methoxy.

In one sub-group of these novel compounds R₃′ may be alkyl, alkenyl, alkynyl, alkoxy, cycloalkoxy, cycloalkylalkoxy, hydroxyalkoxy or alkoxyalkoxy, R₄′ may be halogen, alkyl or alkoxy and R₅′ may be alkyl or alkoxy. Preferably R₃′ is methyl, ethyl, propyl, iso-propyl, butyl, iso-butyl, tert-butyl, pentyl, hexyl, but-1-enyl, pent-1-enyl, but-1-ynyl, pent-1-ynyl, methoxy, ethoxy, propoxy, butoxy, iso-butoxy, 3-methyl-butoxy, pentyloxy, cyclopentyloxy, hexyloxy, cyclopropylmethoxy, cyclobutylmethoxy, 2-hydroxy-ethoxy, 2-methoxy-ethoxy, R₄′ is chloro, bromo, methyl or methoxy, and R₅′ is methyl or methoxy.

In another sub-group of these novel compounds R₃′ may be alkoxy and R₄′ and R₅′ together with the phenyl ring to which they are attached, may form an optionally substituted naphthalene, tetrahydronaphthalene, indane, 1H-indene, isoquinoline, dihydro-benzo[1,4]dioxine or benzo[1,3]dioxole ring system. Preferably R₃′ is propoxy and R₄′ and R₅′ together with the phenyl ring to which they are attached, form a naphthalene-1-yl, indan-4-yl, isoquinolin-5-yl, isoquinolin-8-yl, 1,2,3,4-tetrahydroisoquinolin-8-yl, 2-alkoxycarbonyl-1,2,3,4-tetrahydroisoquinolin-8-yl or 5,6,7,8-tetrahydronaphthalen-1-yl moiety.

In a preferred sub-group of these novel compounds R₃′ is methoxy, propoxy, cyclopentyloxy, pent-1-ynyl or ethanesulfonyloxy;

R₄′ is methyl;

R₅′ is hydroxy, methyl, pentyl, methoxy, propoxy, 2-methoxyethoxy, 2-hydroxyethoxy, 3-hydroxypropoxy, 4-hydroxybutoxy, 2,3-dihydroxypropoxy, 4-acetoxybutoxy, carboxymethoxy, 3-carboxypropoxy, 4-carboxybutoxy, 3-carboxy-2-hydroxypropoxy, 3-carboxy-2,3-dihydroxypropoxy, ethoxycarbonylmethoxy, 3-ethoxycarbonylpropoxy, 4-ethoxycarbonylbutoxy, 3-ethoxycarbonyl-2-hydroxypropoxy, 3-ethoxycarbonyl-2,3-dihydroxypropoxy, carbamoylmethoxy 3-N-ethylcarbamoylpropoxy, 4-N-ethylcarbamoylbutoxy, 2-N,N-dimethylaminoethoxy, 3-N,N-dmethylaminopropoxy, 2-(morpholin-4-yl)-ethyoxy, 2-(piperidin-1-yl)-ethoxy, 3-(morpholin-4-yl)-carbonylpropoxy, 4-(morpholin-4-yl)-carbonylbutoxy, 2,2-dimethyl[1,3]dioxolan-4-ylmethoxy or 2,2-dimethyl-4-carboxy[1,3]dioxolan-5-ylmethoxy; or

R₄′ and R₅′ together with the phenyl ring to which they are attached, form a naphthalen-1-yl, 5,6,7,8-tetrahydronaphthalen-1-yl, indan-4-yl, 1,2,3,4,-tetraisoquinolin-8-yl or 2-tert-butoxycarbonyl-1,2,3,4,-tetraisoquinolin-8-yl moiety.

Preferred novel compounds include
1-phenyl-4-(4-propoxy-naphthalen-1-ylmethylene)-pyrazolidine-3,5-dione;
4-[1-[3-methyl-2-(2-piperidin-1-yl-ethoxy)-4-propoxy-phenyl]-methylidene]-1-phenyl-pyrazolidine-3,5-dione and its hydrochloride salt;
4-(2,4-dimethoxy-3-methyl-benzylidene)-1-phenyl-pyrazolidine-3,5-dione;
4-(2,3-dimethyl-4-ethanesulfonyloxy-benzylidene)-1-phenyl-pyrazolidine-3,5-dione;
4-(2,3-dimethyl-4-propoxy-benzylidene)-1-pyridin-2-yl-pyrazolidine-3,5-dione;
4-(2,3-dimethyl-4-propoxy-benzylidene)-pyrazolidine-3,5-dione;
4-(2,3-dimethyl-4-methoxybenzylidene)-1-phenyl-pyrazolidine-3,5-dione;
4-(2,3-dimethyl-4-pent-1-ynyl-benzylidene)-1-phenyl-pyrazolidine-3,5-dione;
4-(4-propoxy-naphthalen-1-ylmethylene)-pyrazolidine-3,5-dione;
4-[4-(2,3-dimethyl-4-propoxy-benzylidene)-3,5-dioxo-pyrazolidin-1-yl]-benzonitrile;
4-[1-(2-hydroxy-3-methyl-4-propoxy-phenyl)-methylidene]-1-phenyl-pyrazolidine-3,5-dione;
4-(2,3-dimethyl-4-pentyl-benzylidene)-1-phenyl-pyrazolidine-3,5-dione;
4-(2,3-dimethyl-4-pentyl-benzylidene)-pyrazolidine-3,5-dione;
1-(2-chloro-phenyl)-4-(2,3-dimethyl-4-propoxy-benzylidene)-pyrazolidine-3,5-dione;
4-(2,3-dimethyl-4-pent-1-ynyl-benzylidene)-pyrazolidine-3,5-dione;
4-(2,3-dimethyl-4-propoxy-benzylidene)-1-pyridin-4-yl-pyrazolidine-3,5-dione;
4-(2,3-dimethyl-4-propoxy-benzylidene)-1-(4-methyl-phenyl)-pyrazolidine-3,5-dione;
4-(4-cyclopentyloxy-2,3-dimethyl-benzylidene)-pyrazolidine-3,5-dione;
4-(4-cyclopentyloxy-2,3-dimethyl-benzylidene)-1-phenyl-pyrazolidine-3,5-dione;
4-(2,3-dimethyl-4-propoxy-benzylidene)-1-(4-methoxy-phenyl)-pyrazolidine-3,5-dione;
1-(4-bromo-phenyl)-4-(2,3-dimethyl-4-propoxy-benzylidene)-pyrazolidine-3,5-dione;
4-(2,3-dimethyl-4-propoxy-benzylidene)-1-(2-methyl-phenyl)-pyrazolidine-3,5-dione;
4-(2,3-dimethyl-4-propoxy-benzylidene)-1-methyl-pyrazolidine-3,5-dione;
ethyl 4-[4-(2,3-dimethyl-4-propoxy-benzylidene)-3,5-dioxo-pyrazolidin-1-yl)-benzoate; and
1-acetyl-4-[1-(2,3-dimethyl-4-propoxy-phenyl)-methylidene]-pyrazolidine-3,5-dione.

Particularly preferred novel compounds include
1-phenyl-4-[1-(5-propoxy-isoquinolin-8-yl)-methylidene]-pyrazolidine-3,5-dione;
4-[1-[2-(4-ethylamino-4-oxo-butoxy)-3-methyl-4-propoxy-phenyl]-methylidene]-1-phenyl-pyrazolidine-3,5-dione;
4-[1-[2-(4-ethoxycarbonyl-butoxy)-3-methyl-4-propoxy-phenyl]-methylidene]-1-phenyl-pyrazolidine-3,5-dione;
4-[1-[2-((2R)-3-ethoxycarbonyl-2-hydroxy-propoxy)-3-methyl-4-propoxy-phenyl]-methylidene]-1-phenyl-pyrazolidine-3,5-dione;
4-[1-[2-(4-acetoxy-butoxy)-3-methyl-4-propoxy-phenyl]-methylidene]-1-phenyl-pyrazolidine-3,5-dione;
4-[1-[2-(3-ethoxycarbonyl-propoxy)-3-methyl-4-propoxy-phenyl]-methylidene]-1-phenyl-pyrazolidine-3,5-dione;
4-[1-[2-(2-methoxy-ethoxy)-3-methyl-4-propoxy-phenyl]-methylidene]-1-phenyl-pyrazolidine-3,5-dione;
4-[1-[3-methyl-2-(5-morpholin-4-yl-5-oxo-pentyloxy)-4-propoxy-phenyl]-methylidene]-1-phenyl-pyrazolidine-3,5-dione;
1-phenyl-4-[1-(8-propoxy-isoquinolin-5-yl)-methylidene]-pyrazolidine-3,5-dione;
4-[1-[2-(2-amino-2-oxo-ethyloxy)-3-methyl-4-propoxy-phenyl]-methylidene]-1-phenyl-pyrazolidine-3,5-dione;
4-[1-[3-methyl-2-(2-morpholin-4-yl-ethoxy)-4-propoxy-phenyl]-methylidene]-1-phenyl-pyrazolidine-3,5-dione and its hydrochloride salt;
4-[1-[2-(2,2-dimethyl-[1,3]dioxolan-4-ylmethoxy)-3-methyl-4-propoxy-phenyl]-methylidene]-1-phenyl-pyrazolidine-3,5-dione;
4-(2,3-dimethyl-4-propoxy-benzylidene)-1-phenyl-pyrazolidine-3,5-dione;
4-[1-[2-((4R,5S)-4-carboxy-2,2-dimethyl-[1,3]dioxolan-5-ylmethoxy)-3-methyl-4-propoxy-phenyl]-methylidene]-1-phenyl-pyrazolidine-3,5-dione
4-[1-[2-((4R)-2,2-dimethyl-[1,3]dioxolan-4-ylmethoxy)-3-methyl-4-propoxy-phenyl]-methylidene]-1-phenyl-pyrazolidine-3,5-dione;
4-[1-[2-(3-dimethylamino-propoxy)-3-methyl-4-propoxy-phenyl]-methylidene]-1-phenyl-pyrazolidine-3,5-dione and its hydrochloride salt;
1-phenyl-4-[1-(7-propoxy-indan-4-yl)-methylidene]-pyrazolidine-3,5-dione;
1-phenyl-4-(4-propoxy-5,6,7,8-tetrahydro-naphthalen-1-ylmethylene)-pyrazolidine-3,5-dione;
1-phenyl-4-[1-(5-propoxy-1,2,3,4-tetrahydro-isoquinolin-8-yl)-methylidene]-pyrazolidine-3,5-dione and its formate salt;
4-[1-[2-(2-dimethylamino-ethoxy)-3-methyl-4-propoxy-phenyl]-methylidene]-1-phenyl-pyrazolidine-3,5-dione and its hydrochloride salt;
4-[1-[2-(carboxy-methoxy)-3-methyl-4-propoxy-phenyl]-methylidene]-1-phenyl-pyrazolidine-3,5-dione;
4-[1-[2-((4S)-2,2-dimethyl-[1,3]dioxolan-4-ylmethoxy)-3-methyl-4-propoxy-phenyl]-methylidene]-1-phenyl-pyrazolidine-3,5-dione;
4-(3-methyl-2,4-dipropoxy-benzylidene)-pyrazolidine-3,5-dione;
4-[1-(3-methyl-2,4-dipropoxy-phenyl)-methylidene]-1-phenyl-pyrazolidine-3,5-dione;
4-(4-propoxy-5,6,7,8-tetrahydro-naphthalen-1-ylmethylene)-pyrazolidine-3,5-dione;
4-[1-(2-methoxy-3-methyl-4-propoxy-phenyl)-methylidene]-1-phenyl-pyrazolidine-3,5-dione;
4-(2-methoxy-3-methyl-4-propoxy-benzylidene)-pyrazolidine-3,5-dione;
4-(7-propoxy-indan-4-ylmethylene)-pyrazolidine-3,5-dione;
4-[1-(2-tert-butoxycarbonyl-5-propoxy-1,2,3,4-tetrahydro-isoquinolin-8-yl)-methylidene]-1-phenyl-pyrazolidine-3,5-dione; and
4-(2,3-dimethyl-4-propoxy-benzylidene)-1-(4-fluoro-phenyl)-pyrazolidine-3,5-dione.

Most preferred novel compounds include
4-[1-[2-((2S)-3-carboxy-2-hydroxy-propoxy)-3-methyl-4-propoxy-phenyl]-methylidene)-1-phenyl-pyrazolidine-3,5-dione;
4-[1-[2-((2R)-3-carboxy-2-hydroxy-propoxy)-3-methyl-4-propoxy-phenyl]-methylidene]-1-phenyl-pyrazolidine-3,5-dione;
4-[1-(2-(3-carboxy-propoxy)-3-methyl-4-propoxy-phenyl]-methylidene]-1-phenyl-pyrazolidine-3,5-dione;
4-[1-[2-(4-carboxy-butyloxy)-3-methyl-4-propoxy-phenyl]-methylidene]-1-phenyl-pyrazolidine-3,5-dione;
4-[1-[2-(2,3-dihydroxy-propoxy)-3-methyl-4-propoxy-phenyl]-methylidene]-1-phenyl-pyrazolidine-3,5-dione;
4-(1-[2-((2S)-2,3-dihydroxy-propoxy)-3-methyl-4-propoxy-phenyl]-methylidene]-1-phenyl-pyrazolidine-3,5-dione;
4-[1-[2-((2R)-2,3-dihydroxy-propoxy)-3-methyl-4-propoxy-phenyl]-methylidene]-1-phenyl-pyrazolidine-3,5-dione;
4-[1-[2-((2S,3R)-3-carboxy-2,3-dihydroxy-propoxy)-3-methyl-4-propoxy-phenyl]-methylidene]-1-phenyl-pyrazolidine-3,5-dione;
4-[1-[2-(5-ethylamino-5-oxo-pentyloxy)-3-methyl-4-propoxy-phenyl]-methylidene]-1-phenyl-pyrazolidine-3,5-dione;
4-[1-[2-(3-hydroxy-propoxy)-3-methyl-4-propoxy-phenyl]-methylidene]-1-phenyl-pyrazolidine-3,5-dione;
4-[1-[2-(4-hydroxy-butoxy)-3-methyl-4-propoxy-phenyl]-methylidene]-1-phenyl-pyrazolidine-3,5-dione;
4-[1-[2-(2-hydroxy-ethoxy)-3-methyl-4-propoxy-phenyl]-methylidene]-1-phenyl-pyrazolidine-3,5-dione;
4-[1-[2-((2S,3R)-2,3-dihydroxy-3-ethoxycarbonyl-propoxy)-3-methyl-4-propoxy-phenyl]-methylidene]-1-phenyl-pyrazolidine-3,5-dione;
4-[1-[2-(ethoxycarbonyl-methoxy)-3-methyl-4-propoxy-phenyl]-methylidene]-1-phenyl-pyrazolidine-3,5-dione; and
4-[1-[3-methyl-2-(4-morpholin-4-yl-4-oxo-butoxy)-4-propoxy-phenyl]-methylidene]-1-phenyl-pyrazolidine-3,5-dione.

In accordance with the present invention the aforementioned novel compounds can be manufactured by condensing a pyrazolidinedione of the general formula (IV), as shown in the following reaction scheme, 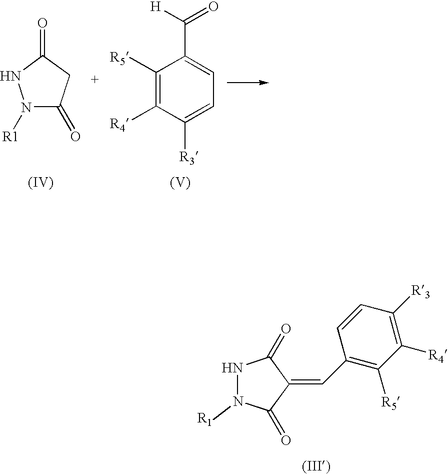
with an aldehyde of the above general formula (V), any reactive group which may be present in the compounds of formulae IV and V being appropriately protected, and, if necessary, splitting off from the condensation product any protecting group(s) which may be undesired.

Some of the starting materials of the above general formulae (IV) and (V) are novel and also form part of the present invention.

These novel starting materials include the following pyrazolidinediones of the general formula (IV):
1-pyridin-2-yl-pyrazolidine-3,5-dione;
4-(3,5-dioxo-pyrazolidin-1-yl)-benzonitrile; and
1-pyridin-4-yl-pyrazolidine-3,5-dione.
   and the following aldehydes of the general formula (V):
4-cyclopentyloxy-2,3-dimethyl-benzaldehyde;
4-propoxy-5,6,7,8-tetrahydro-naphthalene-1-carbaldehyde;
2,3-dimethyl-4-pent-1-ynyl-benzaldehyde;
2,3-dimethyl-4-pentyl-benzaldehyde;
7-propoxy-indan-4-carbaldehyde;
5-propoxy-isoquinoline-8-carbaldehyde;
8-propoxy-isoquinoline-5-carbaldehyde;
2-tert-butyloxycarbonyl-8-formyl-5-propoxy-1,2,3,4-tetrahydroisoquinoline;
2,3-dimethyl-4-ethanesulfonyloxybenzaldehyde;
2-hydroxy-3-methyl-4-propoxy-benzaldehyde;
2-methoxy-3-methyl-4-propoxy-benzaldehyde;
2-(2-methoxy-ethoxy)-3-methyl-4-propoxy-benzaldehyde;
2-(2-hydroxy-ethoxy)-3-methyl-4-propoxy-benzaldehyde;
2-(3-hydroxy-propoxy)-3-methyl-4-propoxy-benzaldehyde;
2-(4-acetoxy-butoxy)-3-methyl-4-propoxy-benzaldehyde;
2-(4-hydroxy-butoxy)-3-methyl-4-propoxy-benzaldehyde;
ethyl (6-formyl-2-methyl-3-propoxy-phenoxy)-acetate;
(6-formyl-2-methyl-3-propoxy-phenoxy)-acetic acid;
2-(6-formyl-2-methyl-3-propoxy-phenoxy)-acetamide;
ethyl 4-(6-formyl-2-methyl-3-propoxy-phenoxy)-butanoate;
4-(6-formyl-2-methyl-3-propoxy-phenoxy)-butanoic acid;
4-(6-formyl-2-methyl-3-propoxy-phenoxy)-butanoic acid ethylamide;
3-methyl-2-(4-morpholin-4-yl-4-oxo-butoxy)-4-propoxy-benzaldehyde;
ethyl 5-(6-formyl-2-methyl-3-propoxy-phenoxy)-pentanoate;
5-(6-formyl-2-methyl-3-propoxy-phenoxy)-pentanoic acid;
5-(6-formyl-2-methyl-3-propoxy-phenoxy)-pentanoic acid ethylamide;
3-methyl-2-(5-morpholin-4-yl-5-oxo-pentyloxy)-4-propoxy-benzaldehyde;
2-(2-dimethylamino-ethoxy)-3-methyl-4-propoxy-benzaldehyde and its hydrochloride;
3-methyl-2-(2-morpholin-4-yl-ethoxy)-4-propoxy-benzaldehyde and its hydrochloride salt;
3-methyl-2-(2-piperidin-1-yl-ethoxy)-4-propoxy-benzaldehyde and its hydrochloride salt;
2-(3-dimethylamino-propoxy)-3-methyl-4-propoxy-benzaldehyde and its hydrochloride salt;
2-(2,2-dimethyl-[1,3]dioxolan-4-ylmethoxy)-3-methyl-4-propoxy-benzaldehyde;
2-((4R)-2,2-dimethyl-[1,3]dioxolan-4-ylmethoxy)-3-methyl-4-propoxy-benzaldehyde;
2-((4S)-2,2-dimethyl-[1,3]dioxolan-4-ylmethoxy)-3-methyl-4-propoxy-benzaldehyde;
ethyl (3R)-4-(6-formyl-2-methyl-3-propoxy-phenoxy)-3-hydroxy-butanoate;
(3R)-4-(6-formyl-2-methyl-3-propoxy-phenoxy)-3-hydroxy-butanoic acid;
ethyl (3S)-4-(6-formyl-2-methyl-3-propoxy-phenoxy)-3-hydroxy-butanoate;
(3S)-4-(6-formyl-2-methyl-3-propoxy-phenoxy)-3-hydroxy-butanoic acid;
methyl (4R,5S)-5-(6-formyl-2-methyl-3-propoxy-phenoxymethyl)-2,2-dimethyl-[1,3]dioxolane-4-carboxylate;
(4R,5S)-5-(6-formyl-2-methyl-3-propoxy-phenoxymethyl)-2,2-dimethyl-[1,3]dioxolane-4-carboxylic acid.

Unless explicitly stated otherwise, the general terms and names used hereinbefore and hereinafter preferably have within the context of this disclosure the following meanings:

The term “alkyl”, as used herein, alone or in any combination, refers to a saturated aliphatic group including a straight or branched hydrocarbon chain containing 1-8 carbon atoms. Representative examples of alkyl include, but are not limited to, methyl, ethyl, n-propyl, iso-propyl, n-butyl, tert-butyl, iso-butyl (or 2-methylpropyl), cyclopropylmethyl, n-pentyl, iso-pentyl, iso-amyl, n-amyl, n-hexyl, n-heptyl, n-octyl and the like. The alkyl group can be optionally substituted with one or more substituents, each independently selected from alkenyl, alkoxy, alkoxycarbonyl, alkylcarbonyl, alkylcarbonyloxy, alkylendioxy, alkylsulfinyl, alkylsulfonyl, alkylthio, alkynyl, amino, aminocarbonyl, aryl, arylalkenyl, arylalkyloxy, aryloxy, aryloxycarbonyl, arylsulfinyl, arylsulfonyl, arylthio, carboxy, cyano, formyl, halogen, haloalkoxy, heterocyclyl, hydroxy, mercapto, nitro, and the like, appended to any carbon atom of the alkyl moiety.

The term “lower alkyl”, as used herein, alone or in any combination, refers to alkyl groups with 1-4 carbon atoms. Representative examples of lower alkyl include, but are not limited to, methyl, ethyl, n-propyl, iso-propyl, n-butyl, iso-butyl, tert-butyl and the like.

The term “alkenyl”, as used herein, alone or in any combination, refers to a straight or branched hydrocarbon chain containing 2-8 carbon atoms with at least one carbon-carbon double bond (RₐR_{b}C═CR_{c}R_{d}). Rₐ-R_{d} refer to substituents, each individually and independently selected from hydrogen and alkyl, alkoxy, alkoxyalkyl and the like. Representative examples of alkenyl include, but are not limited to, ethenyl, 2-propenyl, 2-methyl-2-propenyl, 3-butenyl, 4-pentenyl, 5-hexenyl and the like.

The term “alkylenedioxy”, as used herein, alone or in any combination, refers to a —O(CH₂)ₙO— group, wherein n is preferably 1 or 2, and wherein the oxygen atoms are appended to two adjacent carbon atoms of the parent molecular moiety. Representative examples of alkylenedioxy include, but are not limited to, methylenedioxy, ethylenedioxy, and the like.

The term “alkynyl”, as used herein, alone or in any combination, refers to a straight or branched hydrocarbon chain containing 2-8 carbon atoms with at least one carbon-carbon triple bond (Rₐ—C≡C—R_{b}, Rₐ and R_{b} referring to substituents, each individually and independently selected from hydrogen and alkyl, alkenyl, alkoxy, alkoxyalkyl, and the like). Representative examples of alkynyl include, but are not limited to, acetylenyl, 1-propynyl, 2-propynyl, 1-butynyl, 3-butynyl, 2-pentynyl, and the like.

The term “alkoxy”, as used herein, alone or in any combination, refers to an alkyl group appended to the parent molecular moiety through an oxygen bridge. Representative examples of alkoxy include, but are not limited to, methoxy, ethoxy, propoxy, 2-propoxy, butoxy, tert-butoxy, pentyloxy, hexyloxy, and the like.

The term “alkoxyalkyl”, as used herein, alone or in any combination, refers to an alkoxy group appended to the parent molecular moiety through an alkyl group. Representative examples of alkoxyalkyl include, but are not limited to, tert-butoxymethyl, 2-ethoxyethyl, 2-methoxyethyl, methoxymethyl, and the like.

The term “alkoxycarbonyl”, as used herein, alone or in any combination, refers to an alkoxy group appended to the parent molecular moiety through a carbonyl group. Representative examples of alkoxycarbonyl include, but are not limited to, methoxycarbonyl, ethoxycarbonyl, tert-butoxycarbonyl, and the like.

The term “alkoxycarbonylalkyl”, as used herein, alone or in any combination, refers to an alkoxycarbonyl group appended to the parent molecular moiety through an alkyl group. Representative examples of alkoxycarbonylalkyl include, but are not limited to, methoxycarbonylpropyl, ethoxycarbonylbutyl, 2-tert-butoxycarbonylethyl, and the like.

The term “alkylcarbonyl” or “acyl”, as used herein, alone or in any combination, refers to an alkyl group appended to the parent molecular moiety through a carbonyl group. Representative examples of alkylcarbonyl include, but are not limited to, acetyl, 1-oxopropyl, 2,2-dimethyl-1-oxopropyl, 1-oxobutyl, 1-oxopentyl, and the like.

The term “alkylcarbonylalkyl”, as used herein, alone or in any combination, refers to an alkylcarbonyl group appended to the parent molecular moiety through an alkyl group. Representative examples of alkylcarbonylalkyl include, but are not limited to, 2-oxopropyl, 3,3-dimethyl-2-oxopropyl, 3-oxobutyl, 3-oxopentyl and the like.

The term “alkylcarbonyloxy”, as used herein, alone or in any combination, refers to an alkylcarbonyl group appended to the parent molecular moiety through an oxygen bridge. Representative examples of alkylcarbonyloxy include, but are not limited to, acetyloxy, ethylcarbonyloxy, tert-butylcarbonyloxy and the like.

The term “alkylsulfinyl”, as used herein, alone or in any combination, refers to an alkyl group appended to the parent molecular moiety through a sulfinyl group. Representative examples of alkylsulfinyl include, but are not limited to, methylsulfinyl, ethylsulfinyl and the like.

The term “alkylsulfinylalkyl”, as used herein, alone or in any combination, refers to an alkylsulfinyl group appended to the parent molecular moiety through an alkyl group. Representative examples of alkylsulfinylalkyl include, but are not limited to, methylsulfinylmethyl, ethylsulfinylmethyl and the like.

The term “alkylsulfonyl”, as used herein, alone or in any combination, refers to an alkyl group appended to the parent molecular moiety through a sulfonyl group. Representative examples of alkylsulfonyl include, but are not limited to, methylsulfonyl, ethylsulfonyl, and the like.

The term “alkylsulfonylalkyl”, as used herein, alone or in any combination, refers to an alkylsulfonyl group appended to the parent molecular moiety through an alkyl group. Representative examples of alkylsulfonylalkyl include, but are not limited to, methylsulfonylmethyl, ethylsulfonylmethyl and the like.

The term “alkylthio”, as used herein, alone or in any combination, refers to an alkyl group appended to the parent molecular moiety through a thio group. Representative examples of alkylthio include, but are not limited to, methylthio, ethylthio, tert-butylthio, hexylthio and the like.

The term “alkylthioalkyl”, as used herein, alone or in any combination, refers to an alkylthio group appended to the parent molecular moiety through an alkyl group. Representative examples of alkylthioalkyl include, but are not limited to, methylthiomethyl, 2-(ethylthio)ethyl, and the like.

The term “amino”, as used herein, alone or in any combination, refers to a —NRₑR_{f} group, wherein Rₑ and R_{f} are substituents, each individually and independently selected from hydrogen, alkyl, aryl, arylalkyl, acyl, alkylcarbonyl, arylcarbonyl, carbamoyl, ureido, formyl, alkylsulfonyl, arylsulfonyl, and the like. Representative examples of amino include, but are not limited to, dimethylamino, ethylamino, benzyl-(methyl)amino, and the like.

The term “aminoalkyl”, as used herein, alone or in any combination, refers to an amino group appended to the parent molecular moiety through an alkyl group. Representative examples of aminoalkyl include, but are not limited to, aminomethyl, 2-(amino)ethyl, benzyl-(methyl)aminomethyl, dimethylaminomethyl, and the like.

The term “aminocarbonyl” or “amido”, as used herein, alone or in any combination, refers to an amino group appended to the parent molecular moiety through a carbonyl group. Representative examples of aminocarbonyl include, but are not limited to, dimethylaminocarbonyl, benzyl-aminocarbonyl, ethylaminocarbonyl, and the like.

The term “aminocarbonylalkyl”, as used herein, alone or in any combination, refers to an aminocarbonyl group appended to the parent molecular moiety through an alkyl group. Representative examples of aminocarbonylalkyl include, but are not limited to, 2-amino-2-oxoethyl, 2-(benzylamino)-2-oxoethyl, 2-(methylamino)-2-oxoethyl, 4-amino-4-oxobutyl, 4-(dimethylamino)-4-oxobutyl, and the like.

The term “aryl”, as used herein, alone or in any combination, refers to an carbocyclic group having at least one aromatic ring, e.g. phenyl or biphenyl, or multiple condensed ring systems, in which at least one ring is aromatic, e.g. 1,2,3,4-tetrahydronaphthyl, naphthyl, anthryl, phenanthryl, fluorenyl, and the like. The aryl group may be optionally substituted with one or more functional groups individually and independently selected from alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylendioxy, alkylsulfinyl, alkylsulfinylalkyl, alkylsulfonyl, alkylsulfonylalkyl, alkylthio, alkylthioalkyl, alkynyl, amino, aminoalkyl, aminocarbonyl, aminocarbonylalkyl, aryl, arylalkenyl, arylalkyloxy, arylalkyl, aryloxy, aryloxycarbonyl, aryloxycarbonylalkyl, arylsulfinyl, arylsulfinylalkyl, arylsulfonyl, arylsulfonylalkyl, arylthio, arylthioalkyl, carboxy, carboxyalkyl, cyano, cyanoalkyl, formyl, formylalkyl, halogen, haloalkoxy, haloalkyl, heteroaryl, heterocyclyl, hydroxy, hydroxyalkyl, mercapto, nitro, and the like.

The term “arylalkenyl”, as used herein, alone or in any combination, refers to an aryl group appended to the parent molecular moiety through an alkenyl group. The aryl group may be unsubstituted or substituted. Representative examples of arylalkenyl include, but are not limited to, 2-phenylethenyl, 3-phenylpropen-2-yl, 2-naphth-2-ylethenyl, and the like.

The term “arylalkoxy”, as used herein, alone or in any combination, refers to an aryl group appended to the parent molecular moiety through an alkoxy group. The aryl group may be unsubstituted or substituted. Representative examples of arylalkoxy include, but are not limited to, 2-phenylethoxy, 5-phenylpentyloxy, 3-naphth-2-ylpropoxy, and the like.

The term “arylalkyl”, as used herein, alone or in any combination, refers to an aryl group appended to the parent molecular moiety through an alkyl group. The aryl group may be unsubstituted or substituted. Representative examples of arylalkyl include, but are not limited to, benzyl, 2-phenylethyl, 3-phenylpropyl, 2-naphth-2-ylethyl, and the like.

The term “aryloxy”, as used herein, alone or in any combination, refers to an aryl group appended to the parent molecular moiety through an oxygen bridge. The aryl group can be unsubstituted or substituted. Representative examples of aryloxy include, but are not limited to, phenoxy, naphthyloxy, 3-bromophenoxy, 4-chlorophenoxy, 4-methylphenoxy, 3,4-dimethoxyphenoxy, and the like.

The term “carbamoyl”, as used herein, alone or in any combination, refers to a —C(O)NRₑR_{f} group. Rₑ and R_{f} are substituents, each individually and independently selected from hydrogen, alkyl, arylalkyl, and the like.

Similarly, the term “thiocarbamoyl”, as used herein, alone or in any combination, refers to a —C(S)NRₑR_{f} group.

The term “carbonyl”, as used herein, alone or in any combination, refers to a —C(O) group.

The term “carboxy”, as used herein, alone or in any combination, refers to a —CO₂H group.

The term “carboxyalkyl”, as used herein, alone or in any combination, refers to a carboxy group appended to the parent molecular moiety through an alkyl group. Representative examples of carboxyalkyl include, but are not limited to, carboxymethyl, 2-carboxyethyl, 3-carboxypropyl, and the like.

The term “cyano”, as used herein, alone or in any combination, refers to a —C≡N group.

The term “cyanoalkyl”, as used herein, alone or in any combination, refers to a cyano group appended to the parent molecular moiety through an alkyl group. Representative examples of cyanoalkyl include, but are not limited to, cyanomethyl, 2-cyanoethyl, 3-cyanopropyl, and the like.

The term “cycloalkyl”, as used herein, alone or in any combination, refers to a saturated cyclic hydrocarbon moiety containing 3-15 carbon atoms, optionally substituted with one or more groups, each individually and independently selected from alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylendioxy, alkylsulfinyl, alkylsulfinylalkyl, alkylsulfonyl, alkylsulfonylalkyl, alkylthio, alkylthioalkyl, alkynyl, amino, aminoalkyl, aminocarbonyl, aminocarbonylalkyl, aryl, arylalkenyl, arylalkyloxy, arylalkyl, aryloxy, aryloxycarbonyl, aryloxycarbonylalkyl, arylsulfinyl, arylsulfinylalkyl, arylsulfonyl, arylsulfonylalkyl, arylthio, arylthioalkyl, carboxy, carboxyalkyl, cyano, cyanoalkyl, formyl, formylalkyl, halogen, haloalkoxy, haloalkyl, heterocyclyl, hydroxy, hydroxyalkyl, mercapto, nitro, and the like. Representative examples of cycloalkyl include, but are not limited to, cyclopropyl, cyclobutyl, cyclopentyl, cyclohexyl, cycloheptyl, cyclooctyl and the like. In polycyclic cycloalkyl groups one of the distal rings may be aromatic, e.g., 1-indanyl, 2-indanyl, tetrahydronaphthalene, and the like.

The terms “cycloalkenyl” and “cycloalkynyl”, as used herein, alone or in any combination, refer to unsaturated cyclic hydrocarbon moieties containing at least one carbon-carbon double or carbon-carbon triple bond, respectively. Such moieties may be optionally substituted with one or more groups as discussed hereinabove for the cycloalkyl groups.

The term “formyl”, as used herein, alone or in any combination, refers to a —C(O)H group.

The term “formylalkyl”, as used herein, alone or in any combination, refers to a formyl group, appended to the parent molecular moiety through an alkyl group. Representative examples of formylalkyl include, but are not limited to, formylmethyl, 2-formylethyl, and the like.

The term “halo” or “halogen”, as used herein, alone or in any combination, refers to fluorine, bromine, chlorine, and iodine.

The term “haloalkyl”, as used herein, alone or in any combination, refers to an alkyl group having at least one hydrogen atom replaced with a halogen atom. Representative examples of haloalkyl include, but are not limited to, chloromethyl, 2-fluoroethyl, trifluoromethyl, pentafluoroethyl, 2-chloro-3-fluoropentyl, and the like.

The term “haloalkoxy”, as used herein, alone or in any combination, refers to an alkoxy group having at least one hydrogen atom replaced with a halogen atom. Representative examples of haloalkoxy include, but are not limited to, chloromethoxy, 2-fluoroethoxy, trifluoromethoxy, pentafluoroethoxy, and the like.

The term “heterocyclyl”, as used herein, alone or in any combination, refers to a monocyclic, bicyclic or polycyclic ring system containing up to 15 ring atoms, at least one of these being a hetero atom independently selected from nitrogen, oxygen or sulfur. The ring system may be saturated, partially unsaturated, unsaturated or aromatic. Representative examples of heterocyclyl include, but are not limited to, furyl, imidazolyl, imidazolinyl, imidazolidinyl, isothiazolyl, isoxazolyl, morpholinyl, oxadiazolyl, oxazolyl, oxazolinyl, oxazolidinyl, piperazinyl, piperidinyl, pyranyl, pyrazinyl, pyrazolyl, pyridyl, pyrimidinyl, pyridazinyl, pyrrolyl, pyrrrolinyl, pyrrolidinyl, tetrahydrofuranyl, tetrahydrofuranyl, tetrahydrothienyl, thiadiazolyl, thiazolyl, thiazolinyl, thiazolidinyl, thienyl, thiomorpholinyl, 1,1-dioxothiomorpholinyl, benzimidazolyl, benzothiazolyl, benzothienyl, benzoxazolyl, benzofuranyl, indolyl, indolinyl, isobenzofuranyl, isobenzothienyl, isoindolyl, isoindolinyl, isoquinolinyl, quinolinyl, and the like. Defined heterocyclyl moieties may be optionally substituted with one or more groups, each individually and independently selected from alkenyl, alkoxy, alkoxyalkyl, alkoxycarbonyl, alkoxycarbonylalkyl, alkyl, alkylcarbonyl, alkylcarbonylalkyl, alkylcarbonyloxy, alkylendioxy, alkylsulfinyl, alkylsulfinylalkyl, alkylsulfonyl, alkylsulfonylalkyl, alkylthio, alkylthioalkyl, alkynyl, amino, aminoalkyl, aminocarbonyl, aminocarbonylalkyl, aryl, arylalkenyl, arylalkyloxy, arylalkyl, aryloxy, aryloxycarbonyl, aryloxycarbonylalkyl, arylsulfinyl, arylsulfinylalkyl, arylsulfonyl, arylsulfonylalkyl, arylthio, arylthioalkyl, carboxy, carboxyalkyl, cyano, cyanoalkyl, cycloalkyl, formyl, formylalkyl, halogen, haloalkoxy, haloalkyl, heterocyclyl, heteroaryl, hydroxy, hydroxyalkyl, mercapto, nitro, and the like.

The term “heteroaryl”, as used herein, alone or in any combination, is a special case of heterocyclyl and refers to a mono- or bicyclic or polycyclic aromatic ring system, in which at least one heterocyclic ring is aromatic.

The term “heterocyclylalkenyl”, as used herein, alone or in any combination, refers to a heterocyclyl group appended to the parent molecular moiety through an alkenyl group. Representative examples of heterocyclylalkenyl include, but are not limited to, 2-pyrid-3-ylethenyl, 3-quinolin-3-ylpropen-2-yl, 5-pyrid-4-ylpenten-4-yl, and the like.

The term “heterocyclylalkoxy”, as used herein, alone or in any combination, refers to a heterocyclyl group appended to the parent molecular moiety through an alkoxy group. Representative examples of heterocyclylalkoxy include, but are not limited to, 2-pyrid-3-ylethoxy, 3-quinolin-3-ylpropoxy, 5-pyrid-4-ylpentyloxy, and the like.

The term “heterocyclylalkyl”, as used herein, alone or in any combination, refers to a heterocyclyl group appended to the parent molecular moiety through an alkyl group. Representative examples of heterocyclylalkyl include, but are not limited to, 2-pyrid-3-ylmethyl, 2-pyrimidin-2-ylpropyl, and the like.

The term “heterocyclyloxy”, as used herein, alone or in any combination, refers to a heterocyclyl group appended to the parent molecular moiety through an oxy group. Representative examples of heterocyclyloxy include, but are not limited to, pyrid-3-yloxy, quinolin-3-yloxy, and the like.

The term “hydroxy” or “hydroxyl” as used herein, alone or in any combination, refers to an —OH group

The term “hydroxyalkyl”, as used herein, alone or in any combination, refers to an alkyl group having at least one hydrogen atom replaced with a hydroxy group. Representative examples of hydroxyalkyl include, but are not limited to, hydroxymethyl, 2-hydroxyethyl, 3-hydroxypropyl, 2-ethyl-4-hydroxyheptyl, and the like.

The term “nitro”, as used herein, alone or in any combination, refers to a —NO₂ group.

The term “oxo”, as used herein, alone or in any combination, refers to a ═O group.

The term “oxy”, as used herein, alone or in any combination, refers to a —O— group.

The terms “mercapto” and “thiol”, as used herein, alone or in any combination, refer to a —SH group.

The terms “thio”, “sulfinyl”, and “sulfonyl”, as used herein, alone or in any combination, refer to a —S(O)ₙ group with n=0, 1 and 2, respectively.

Within the scope of the present invention, unless indicated otherwise, compounds of formulae (I) and (III), the latter including formula (III′), or pharmaceutically acceptable salts thereof are included that may exist in, and be isolated in, isomeric forms, including cis- or trans isomers or mixtures thereof, and tautomers. Other compounds of this invention may contain one or more stereogenic or asymmetric centers, such as one or more asymmetric carbon atoms, and thus may give rise to optically pure enantiomers, mixtures of enantiomers, racemates, enantiomer-pure diastereomers, mixtures of diastereomers, epimers, and other stereoisomeric forms that may be defined, in terms of absolute stereochemistry, as (R)-, (S)- or (R,S)-configured, preferably in the (R)- or (S)-configuration. Such isomers can be obtained by methods within the knowledge of one skilled in the art, e.g. by stereochemically controlled synthesis using chiral synthons or chiral reagents, or by means of classical separation techniques, such as chromatographic or crystallization methods, or by other methods known in the art, such as through formation of diastereomeric salts, for example by salt formation with an enantiomerically pure chiral acid, or by means of chromatography, for example by using chromatographic materials modified with chiral ligands. Furthermore, the present invention refers to compounds containing centers of any geometric asymmetry, like, for example, unsymmetrically substituted olefinic double bond, including E or Z geometric isomers and mixtures thereof. Generally, pure isomers of compounds of formulae (I) and (III) are preferred over isomeric mixtures.

In the present invention, the compounds of formulae (I) and (III) may be used in the form of pharmaceutically acceptable salts. The term “pharmaceutically acceptable salts” refers to relatively nontoxic, inorganic or organic acid and base addition salts, which retain the biological effectiveness and properties of the parent compound, and which are not biologically or otherwise undesirable (see, e.g., Berge et al., J. Pharm. Sci. 1977, 66, 1-19).

Certain compounds of the present invention can contain one or more basic functional groups, such as amino, alkylamino, or arylamino, and, thus, be capable of forming pharmaceutically acceptable acid addition salts. These acid addition salts may be prepared by standard procedures in a suitable solvent from the parent compound of formula (I) or (III), with an appropriate amount of an inorganic acid, including, but not limited to, for example, hydrochloric acid, hydrobromic acid, sulfuric acid, or phosphoric acid; or of an organic acid, including, but not limited to, acetic acid, propionic acid, octanoic acid, decanoic acid, glycolic acid, pyruvic acid, oxalic acid, maleic acid, malonic acid, succinic acid, fumaric acid, tartaric acid, citric acid, ascorbic acid, amino acids, such as glutamic acid or aspartic acid, benzoic acid, cinnamic acid, salicylic acid, mandelic acid, methanesulfonic acid, ethanesulfonic acid, p-toluenesulfonic acid, or other acidic organic compounds.

Certain compounds of the present invention may, on the other hand, contain one or more acidic functional groups and, thus, be capable of forming pharmaceutically acceptable base addition salts. These salts can be prepared by addition of an appropriate amount, usually in stoichiometric ratio, of an alkaline reagent, such as hydroxide, carbonate or alkoxide, containing the appropriate cation, to the free acid in a suitable solvent. Preferred inorganic salts include, but are not limited to, ammonium, sodium, potassium, calcium or magnesium, also zinc salts and the like. Preferred salts derived from organic bases include, but are not limited to, salts of primary, secondary, and tertiary amines, substituted amines, cyclic amines, and basic ion exchange resins, such as isopropylamine, trimethylamine, diethylamine, triethylamine, tripropylamine, ethanolamine, lysine, arginine, N-ethylpiperidine, piperidine, polyamine resins, and the like.

Compounds of the present invention containing both acidic and basic groups can also form internal salts (zwitter ions).

For isolation or purification purposes, it is also possible to use pharmaceutically unacceptable salts, for example perchlorates, picolinates, picrates, or the like. For therapeutic use, only pharmaceutically acceptable salts or free compounds are employed, where applicable in the form of pharmaceutical preparations, and these are therefore preferred.

Certain compounds of formulae (I) and (III), including their salts, may exist in solvated as well unsolvated forms, such as, for example, hydrated forms, or their crystals may, for example, include the solvent used for crystallization. Different crystalline forms may be present. The present invention encompasses all such solvated and unsolvated forms.

The present invention also relates to prodrug derivatives of the parent compounds of formulae (I) and (III). The term “prodrug” refers to pharmacologically inactive precursors of a drug that may be converted into its therapeutically active form under physiological conditions in vivo, for example, when they undergo solvolysis, or enzymatic degradation in blood, or in cells, (Bundgard H., “Design of Prodrugs”, pp. 7-9, 21-24, Elsevier, Amsterdam (1985); Silverman R. B., “The Organic Chemistry of Drug Design and Drug Action”, pp. 352-401, Academic Press, San Diego, Calif. (1992); Higuchi T. et al., “Pro-drug as Novel Delivery Systems”, A.C.S. Symposium Series, Vol. 14). The term “prodrug” also includes any covalently bonded carriers, which release the active parent compound in vivo when administered to a mammal. Prodrug modifications of a compound often offer advantages of solubility, bioavailability, absorption, tissue compatibility, tissue distribution, or delayed release in the mammalian organism. Prodrugs are variations or derivatives of the compounds of formulae (I) and (III), which have groups cleavable under metabolic conditions, for example, pharmaceutically acceptable esters, or amides. Such groups can be cleaved enzymatically or non-enzymatically, or hydrolytically to the free hydroxy, carboxy, or amino group of the active parent compound. In another embodiment, the prodrug is a reduced form, which is oxidized in vivo to the therapeutic compound, for example, a thiol, which is oxidized to a sulfonate or sulfate, an alcohol to a carboxylic acid.

Further included within the scope of the present invention are pharmaceutically acceptable esters of the compounds of formulae (I) and (III). The term “pharmaceutically acceptable esters” refers to relatively non-toxic, esterified products of the parent compound. These esters can be prepared in situ during the final isolation and purification of the compounds, or by separately reacting the purified compounds in its free acid or hydroxyl form with a suitable esterifying agent. Carboxylic acids can be converted into esters via treatment with an alcohol in the presence of a catalyst. Hydroxyl containing derivatives can be converted into esters via treatment with an esterifying agent such as alkanoyl halides. The term further includes lower hydrocarbon groups capable of being solvated under physiological conditions, for example, alkyl esters, preferred methyl, ethyl, and propyl ester, methoxymethyl ester, methylthiomethyl ester, pivaloyloxymethyl ester and the like (see, e.g., Berge et al., J. Pharm. Sci. 1977, 66, 1-19).

The compounds of the present invention have useful, in particular pharmacologically useful, properties. They are able to specifically antagonize the effect of endogenous ADP on its platelet ADP receptor, the P2Y₁₂ receptor. The platelet ADP receptor P2Y₁₂ upon activation with ADP is responsible for blood platelet aggregation. The compounds of formulae (I) and (III) are therefore useful in treatment or prevention of vascular diseases that respond to the blockade of the P2Y₁₂ receptor.

A compound or a pharmaceutical composition of the invention may be used as a drug (medicine) or therapeutic agent for prevention and/or treatment of peripheral vascular, cardiovascular and cerebrovascular diseases or conditions, associated with platelet aggregation, particularly related to thrombosis in humans and other mammals. Such compounds may be useful as inhibitors of platelet activation, aggregation and degranulation, as anti-thrombotic agents or in the treatment and/or prevention of, for example any thrombosis, particularly platelet-dependent thrombotic indications, including, but not limited to, acute myocardial infarction, unstable angina, coronary angioplasty (PTCA), perithrombolysis, primary arterial thrombotic complications of atherosclerosis such as thrombotic or embolic stroke, chronic stable angina, transient ischemic attacks, strokes, peripheral vascular disease, myocardial infarction with or without thrombolysis, pre-eclampsia/eclampsia, venous thrombosis such as deep venous thrombosis, venoocclusive disease, embolism, disseminated intravascular coagulation, thrombotic thrombocytopenic purpura, heparin-induced thrombocytopenia, hemolytic uremic syndrome, thrombotic complications of septicernia, adult respiratory distress syndrome, anti-phospholipid syndrome, hematological conditions such as myeloproliferative disease, including thrombocythemia; thrombotic and restenoic complications following invasive procedures, for example angioplasty, carotid endarterectomy, post coronary bypass graft surgery, vascular graft surgery, stent placements and insertion of endovascular devices and protheses, thrombotic complications of surgical or mechanical damage such as tissue salvage following accidental or surgical trauma, reconstructive surgery including skin and muscle flaps.

The compounds of the invention may also be used for the prevention of mechanically-induced platelet activation in vivo, for example to prevent microthromboembolism in cardiopulmonary bypass; or in vitro in the preservation of blood products, for example platelet concentrates; or shunt occlusion such as in renal dialysis and plasmapheresis, thrombosis secondary to vascular damage/inflammation such as vasculitis, arteritis, glomerulonephritis, inflammatory bowel disease and organ graft rejection, conditions such as migraine, Raynaud's phenomenon, atheromatous plaque formation/progression, vascular stenosis/restenosis and asthma, in which platelet-derived factors play a role in the disease process.

In another aspect, the compounds of formulae (I) and (III) may be used as standard or reference compounds in tests or assays involving the inhibition of the platelet ADP receptor, P2Y₁₂. Such compounds could be made commercially available for use as a reference, quality standard or control, for example in pharmaceutical research when developing new assays or protocols related to P2Y₁₂ activity.

As mentioned earlier, compounds of formulae (I) and (III), or salts, or prodrugs thereof, antagonize the ADP dependent activation of the platelet ADP receptor P2Y₁₂. The biological effect of such compounds may be tested in a variety of in vitro, ex vivo and in vivo assays.

The ability of the compounds of formulae (I) and (III) to bind to the P2Y₁₂ receptor may be measured by methods similar to those described in Gachet C. et al., Br. J. Hematol. 1995, 91, 434-444 and by the method described below in Example 16.

With this type of assay, IC₅₀ values (i.e. the concentrations where half-maximal inhibition of the interaction is found) in the range of 0.001 to 10 μM, preferably values below 1 μM, in particular values below 0.05 μM, are found with test compounds of formulae (I) and (III). Exemplary IC₅₀ values determined in this test are given below in Example 17.

The ability of the compounds of the invention to inhibit ADP-induced aggregation of platelets may be measured by methods similar to those described in Born G. V. R., and Cross M. J., J. Physiol. 1963, 168, 178-195 and the method described below in Example 18.

With this type of assay, ED₅₀ (i.e. the effective dose where half-maximal inhibition of the aggregation is found) in the range of 0.05 to 5 μM, preferably values below 1 μM, in particular values below 0.1 μM, are found with test compounds of formulae (I) and (III).

A functional assay with cells expressing the human P2Y₁₂ receptor may be used to detect changes in the levels of intracellular calcium concentration following compound treatment. After addition of the compound the cells are challenged with ADP. In a Fluorescent Imaging Plate Reader (FLIPR™, Molecular Devices, Sunnyvale, Calif.) fluorescence emission is recorded during both additions, emission peak values above base level after ADP addition were exported, normalized to low controls (no ADP) and high controls (no active compound). The relative values of the remaining activity were used to determine IC₅₀ values by curve fitting them to a four-parameter logistic sigmoid curve.
Calculation

$$\% remaining activity = \frac{( \% value with compound - 0 \% value )}{( 100 \% value - 0 \% value )} \times 100 \%$$

The ability of the compounds to inhibit ADP induced change of intracellular calcium levels via P2Y₁₂ activation may be measured by methods known of one skilled in the art or by the method described below in Example 19.

With this assay, IC₅₀ values (i.e. the concentration of a compound at which the remaining activity is 50%) in the range of 0.001 and 10 μM, preferably below 0.5 μM, are obtained with test compounds of formulae (I) and (III).

The results of these assays clearly demonstrate, that the present invention provides functional antagonists of the P2Y₁₂ receptor inhibiting platelet aggregation, and therefore may be useful for the treatment of vascular diseases, particularly thrombosis.

On the basis of the biological studies discussed hereinabove, a compound of formula (I) or (III) according to the invention may show therapeutic efficacy against vascular disorders mentioned herein, especially against thrombotic diseases.

A compound of formula (I) or (III), a pharmaceutically acceptable salt or a prodrug thereof, can be administered alone in pure form or in combination with one or more other therapeutic agents, possible combination therapy taking the form of fixed combinations or the administration of a compound of the invention and one or more other therapeutic agents being staggered or given independently of one another, or the combined administration of fixed combinations and one or more other therapeutic agents. A compound of formula (I) or (III) can besides or in addition be administered especially for anti-thrombotic therapy in combination with other vascular diseases. Long-term therapy is equally possible as is adjuvant therapy in the context of other treatment strategies, as described above. Other possible treatments are preventive therapies, for example in patients at risk.

The invention relates also to pharmaceutical compositions comprising compounds of formula (III), to their use in therapeutic, in a broader aspect of the invention also prophylactic treatment or a method of treatment of the diseases mentioned above, to the compounds for said use and to the preparation of pharmaceutical formulations (medicines).

The pharmaceutically acceptable compounds of the present invention may be used, for example, for the preparation of pharmaceutical compositions that comprise an effective amount of the active ingredient together or in admixture with a significant amount of one or more inorganic, organic, solid or liquid, pharmaceutically acceptable carriers.

The invention relates also to a pharmaceutical composition that is suitable for administration to a warm-blooded animal, especially a human (or to cells or cell lines derived from a warm-blooded animal, especially a human, e.g. blood platelets), for the treatment or, in a broader aspect of the invention, prevention of (i.e. prophylaxis against) a disease that responds to blockade of the interaction of the platelet adenosine diphosphate (ADP) receptor with ADP, comprising an amount of a compound of formula (I) or (III) or a pharmaceutically acceptable salt or a prodrug thereof, which is effective for said inhibition, together with at least one pharmaceutically acceptable carrier.

The pharmaceutical compositions according to the invention are those for enteral administration, such as nasal, buccal, rectal, dermal or, especially oral administration, and for parenteral administration, such as intramuscular, intravenous or subcutaneous, intrasternal, intravitreal, injection or infusion, to warm-blooded animals, especially humans. Such compositions comprise an effective dose of the pharmaceutically active ingredient, alone or together with a significant amount of a pharmaceutically acceptable carrier. The dosage of the active ingredient depends on the species of warm-blooded animal, the body weight, the age and the individual conditions, individual pharmacokinetic data, the disease to be treated and the mode of administration.

The invention relates also to a process or a method for the treatment of a pathological condition mentioned hereinabove, especially a disease, which responds to blockade of the interaction of the platelet adenosine diphosphate (ADP) receptor with ADP, especially thrombosis. The compounds of formulae (I) and (III) or salts or a prodrugs thereof can be administered as such or especially in the form of pharmaceutical compositions.

The dose to be administered to warm-blooded animals, for example humans of approximatively 70 kg body weight, is preferably from approximatively 3 mg to approximatively 30 g, more preferably from approximatively 10 mg to approximatively 1000 mg per person per day, divided preferably into 1 to 3 single doses which may, for example, be of the same size. The amount of the compound actually administered will typically be determined by a physician, in the light of the relevant circumstances, including the condition to be treated, the chosen route of administration, the actual compound administered, the age, the weight, and response of the individual patient, the severity of the patient's symptoms, and the like, for example, children usually receive half of the adults dose.

The pharmaceutical compositions comprise from approximately 1% to approximately 95%, preferably from approximately 20% to approximately 90%, active ingredient. Pharmaceutical compositions according to the invention may be, for example, in unit dosage forms such as coated and uncoated tablets, pills, ampoules, vials, suppositories, dragées, or capsules. Further dosage forms are, for example, ointments, creams, pastes, emulsions, foams, chewable gums, tinctures, lip-sticks, drops, sprays or aerosols, syrups or elixirs, dispersions, transdermal patches or pads, or via an intravitreal device that releases the compound in a sustained capacity, and the like. Examples are capsules containing from about 0.05 g to about 1.0 g active ingredient.

The pharmaceutical compositions of the present invention are prepared in a manner known, per se, for example by means of conventional mixing, granulating, coating, dissolving, lyophilizing or confectioning processes.

Solutions of the active ingredient, and also suspensions, and especially isotonic aqueous solutions or suspensions, are preferably used, it being possible, for example in the case of lyophilized compositions, that comprise the active ingredient alone or together with a carrier, for example mannitol, for such solutions or suspensions to be produced prior to use. The pharmaceutical compositions may be sterilized and/or may comprise excipients, for example preservatives, stabilizers, wetting agents and/or emulsifiers, solubilizers, salts for regulating osmotic pressure and/or buffers and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chain fatty acid having from 8 to 22, especially from 12 to 22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, β-carotene or 3,5-di-tert-butyl-4-hydroxytoluene. The alcohol component of those fatty acid esters has a maximum of 6 carbon atoms and is mono- or poly-hydroxy, for example a mono-, di- or trihydroxy, alcohol, for example methanol, ethanol, propanol, butanol, or pentanol or the isomers thereof, but especially glycol and glycerol. The following examples of fatty acid esters are therefore to be mentioned: ethyl oleate, iso-propyl myristate, iso-propyl palmitate, “Labrafil M2375” (polyoxyethylene glycerol trioleate, Gattefosse, Paris), “Miglyol 812” (triglyceride of saturated fatty acids with chain length of C8 to C12, Hüls AG, Germany), but especially vegetable oils, such as cottonseed oil, almond oil, olive oil, castor oil, sesame oil, soybean oil and more especially groundnut oil.

The injection or infusion compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice, or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum Arabic, talc, polyvinylpyrrolidone, polyethylene glycol, and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and of soft sealed capsules made of gelatine and a plasticiser, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oil excipients, such as fatty oils, paraffin oil or liquid polyethylene glycols, it being possible also for stabilizers and/or antibacterial agents to be added. Dyes or pigments may be added to the tablets or dragée coatings or the capsule casings, for example for identification purposes or to indicate different doses of active ingredient.

For parenteral administration, aqueous solutions of an active ingredient in water-soluble form, for example of a water-soluble salt, or aqueous injection suspensions that contain viscosity-increasing substances and stabilizers, are especially suitable. The active ingredient, optionally together with excipients, can also be in the form of a lyophilizate and be made into a solution before parenteral administration by the addition of solvents.

Compounds of the invention may be manufactured by the application or adaptation of known methods, by which is meant methods used heretofore or described in the literature, for example those described by Larock R. C. in “Comprehensive organic transformations: a guide to functional group preparations”, VCH publishers, 1999.

In the reactions described hereinafter, it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for example see Greene T. W. and Wuts P. G. M. in “Protective groups in organic synthesis” Wiley-Interscience, 1999.

Generally, alkylidene pyrazolidinediones of formula (I) may be prepared by the condensation of pyrazolidinedione of formula (IV) with an aldehyde of formula (V), optionally in the presence of a suitable base such as pyridine, piperidine, diisopropylethylamine, triethylamine, in a suitable solvent, such as ethanol, methanol, 1-butanol or acetic acid as shown in Scheme 1, hereinbelow. The preferred conditions are heating the pyrazolidinedione of formula (IV) with an aldehyde of formula (V) in ethanol between 60 to 80° C.

Certain R₂ moieties in a structure of formula (I), obtained in step a, such as moieties containing a reactive substituent which is protected, might allow further synthetic modifications (step b), such as a deprotection step, to give a compound of formula (I′).

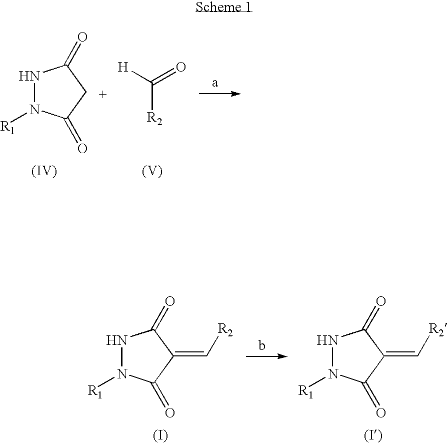

Thus, for example, the tert-butoxycarbonyl group present in 4-[1-(2-tert-butoxycarbonyl-5-propoxy-1,2,3,4-tetrahydro-isoquinolin-8-yl)-methylidene]-1-phenyl-pyrazolidine-3,5-dione can be removed by means of HCl to give 1-phenyl-4-[1-(5-propoxy-1,2,3,4-tetrahydro-isoquinolin-8-yl)-methylidene]-pyrazolidine-3,5-dione; or the ketal group present in 4-[1-[2-(2,2-dimethyl-[1,3]dioxolan-4-ylmethoxy)-3-methyl-4-propoxy-phenyl]-methylidene]-1-phenyl-pyrazolidine-3,5-dione can be removed by means of HCl to give 4-[1-[2-(2,3-dihydroxy-propoxy)-3-methyl-4-propoxy-phenyl]-methylidene]-1-phenyl-pyrazolidine-3,5-dione.

Pyrazolidinediones of formula (IV) can be prepared by a method which is analogous to the method described by Conrad M. and Zart A., Ber. 1906, 2282-2288, and shown in Scheme 2, hereinbelow, in a cyclocondensation of a hydrazine of formula 1 with a malonic acid derivative of formula 2, whereby LG₁ and LG₂ each represent an appropriate leaving group such as halogen, preferably chloro; or alkoxy, preferably methoxy or ethoxy; or aryloxy; in a suitable solvent, such as methanol or ethanol; in the presence or absence of a base such as sodium methoxide or sodium ethoxide.

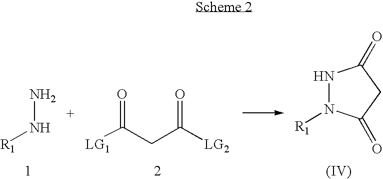

In another preferred method, a compound of formula (IV) can be prepared by cyclizing a compound of formula 3 by a method, which is analogous to the method described by Michaelis A. and Burmeister R., Ber. 1892, 1502-1513, as shown in Scheme 3, hereinbelow; in the presence of an appropriate base such as sodium hydroxide, potassium hydroxide, sodium methoxide, sodium ethoxide, potassium carbonate, or sodium hydride; in a suitable solvent like an alcohol, such as ethanol, or methanol; tetrahydrofuran; or N,N-dimethylformamide.

Under preferred conditions, a compound of formula 3 cyclizes by dissolving such a compound in a solvent such as ethanol and stirring the solution at room temperature in the presence of sodium hydroxide.

Preferably, compounds of formula 3 are prepared by the condensation of hydrazines of formula 1 with malonic acid derivatives of formula 2,
whereby LG₁ is a leaving group such as OH; and LG₂ represents an alkoxy, preferably methoxy or ethoxy; or an aryloxy group in the presence of a coupling reagent, such as 1,3-dicyclohexylcarbodiimide, O-(7-azabenzotriazol-1-yl)-N,N,N′,N′-tetramethyluronium hexafluorophosphate; or a halogen, preferably chloro; in the presence of a base such as triethylamine, N,N-diisopropylethylamine or pyridine; in a suitable solvent such as tetrahydrofuran, dioxane or N,N-dimethylformamide; at low temperatures, preferably below room temperature.

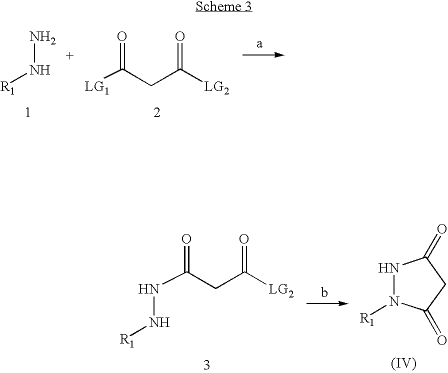

Alternative methods for the preparation of pyrazolidinediones of formula (IV) are:
Condensation of hydrazines of formula 1 with malonyl dichloride in a suitable solvent such as THF, by a method, which is analogous to the method described in WO 02/102359.
Condensation of N′-acetylhydrazines with malonic acid (i.e. compound of formula 2, whereby LG₁ and LG₂, both represent hydroxy), in the presence of phosphorous oxychloride, by a method, which is analogous to the method described by Michaelis A. and Schenk K., Ber. 1907, 3568-3569.
Condensation of hydrazines of formula 1 with 1,2-propadiene-1,3-dione, by a method, which is analogous to the method described by van Alphen J., Recl. Trav. Chim. Pays-Bas 1924, 823-866.
Condensation of hydrazines of formula 1 with ethyl cyanoacetate followed by acid hydrolysis, by a method, which is analogous to the method described by Weissberger A. and Porter H. D., J. Am. Chem. Soc. 1943, 52-54.

Aldehydes of formula (V) are commercially available or may be prepared according to methods known to those skilled in the art or by methods described hereinafter.

A preferred embodiment of the present invention provides the preparation of tri-substituted benzaldehydes of formula (V) as shown in Scheme 4, hereinbelow, wherein R₃ represents alkoxy, preferably propoxy; and
R₄ and R₅ are as defined hereinabove. Such tri-substituted benzaldehyde can be obtained as depicted in Scheme 4, starting from the corresponding tri-substituted benzene of formula 4b, which is brominated by means of N-bromosuccinimide in acetonitrile at room temperature to yield the bromobenzene derivative of formula 5 (step a), by a method which is analogous to the method described by Carreno M. C. et al., J. Org. Chem. 1995, 5328-5331. In a second step (step b), the bromobenzene derivative of formula 5 is first transformed into the corresponding aryl-lithium intermediate by using an alkyl lithium reagent, preferably butyllithium, in a solvent like tetrahydrofuran, at a temperature below 0° C., preferably at −78° C., and then quenching with a dialkylformamide, such as N,N-dimethylformamide, or an alkyl formate, such as methyl formate, to yield the aldehyde of formula (V).

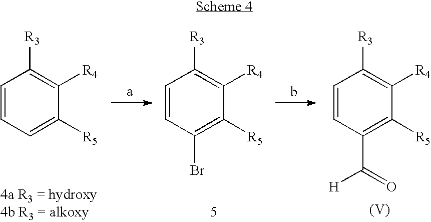

Another embodiment of the present invention provides the preparation of tri-substituted benzene derivatives of formula 4b, wherein R₃ is alkoxy, preferably propoxy; and R₄ and R₅, together with the phenyl ring to which they are attached, form a bicyclic, optionally substituted carbocyclic or heterocyclic ring system; by alkylating the corresponding phenol of formula 4a, wherein R₃ represents hydroxy; with an alkylhalide, preferably 1-bromopropane; in a solvent such as N,N-dimethylformamide; in the presence of a base, such as potassium carbonate, or sodium hydride; at elevated temperatures between 30° C. and 100° C.

Particularly preferred examples of bicyclic compounds of formula 4b, are depicted hereinbelow:

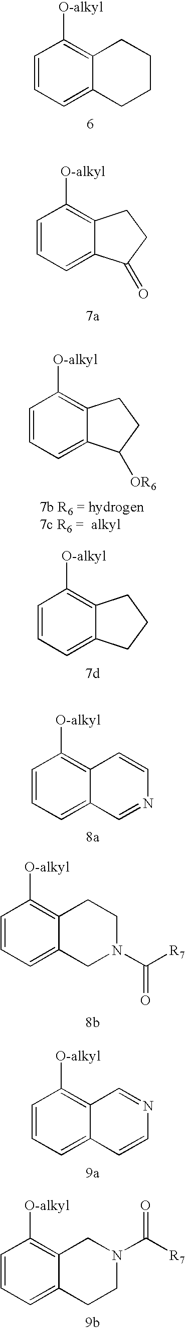

A benzyl alcohol of formula 7b, wherein R₆ represents hydrogen; can be obtained by reducing a ketone of formula 7a by means of a reducing agent, such as sodium borohydride, in a solvent such as an alcohol, preferably methanol.

A benzyl alcohol of formula 7b, wherein R₆ represents hydrogen, can be reduced, e.g. by hydrogenation in the presence of a metal catalyst, such as palladium on charcoal, to give a compound of formula 7d, which is a representative member of the aforementioned class of tri-subsubstituted benzenes of formula 4b.

A benzyl alcohol of formula 7b (R₆ representing hydrogen) may be further modified by treating with an alkylhalide in the presence of a base to give a compound of formula 7c, wherein R₆ represents alkyl, representing an example of the aforementioned tri-subsubstituted benzenes of formula 4b.

Reduction of the pyridine ring in isoquinoline derivatives of formulae 8a and 9a, e.g. by hydrogenation in the presence of a metal catalyst, such as platinium oxide, yields the corresponding 5-alkoxy-1,2,3,4-tetrahydro-isoquinoline and, respectively, 8-alkoxy-1,2,3,4-tetrahydro-isoquinoline, which after treatment with a —CO—R₇ transferring reagent, such as e.g. carboxylic acid halides or anhydrides, or carboxylic acids in the presence of a coupling reagent, or chloroformates in the presence of a base, leads to tetrahydro-isoquinoline derivatives of formulae 8b and 9b, respectively, wherein R₇ represents preferably alkoxycarbonyl or alkylcarbonyl.

Treatment of the aforesaid 5-alkoxy-1,2,3,4-tetrahydro-isoquinoline or 8-alkoxy-1,2,3,4-tetrahydro-isoquinoline with an alkyl halide or with an alkylsulfonyl halide yields compounds corresponding to formula 8b and, respectively, 9b but wherein the nitrogen atom carries an alkyl or alkylsulfonyl group instead of the group —CO—R₇.

An isoquinoline derivative of formula 9a, which is a representative of the aforementioned tri-subsubstituted benzenes of formula 4b, may be prepared from 2-alkoxy-benzaldehyde by a method which is analogous to the procedure described by Hendrickson J. B. et al., J. Org. Chem. 1983, 3346-3347.

Representatives of a subgroup of benzaldehydes of formula (V) are represented by formulae (VI) and (VI′), as shown in Scheme 6, hereinbelow, wherein R₈ and R₉ each represent alkyl and R₄ represents methyl. Such compounds can be prepared as depicted in Scheme 6.

A phenol of formula 10 is converted to a benzaldehyde of formula 11 in a well-known formylation reaction such as the Vilsmeier formylation (step a), according to Nielsen S. F. et al., J. Med. Chem. 1998, 4819-4832. Subsequent alkylation of the hydroxy group in para-position to the formyl group can be achieved by reacting an aldehyde of formula 11 with an R₈-transferring reagent, such as an alkylhalide, alkylmesylate or alkyltosylate, preferably 1-bromopropane, in a solvent like acetonitrile, in the presence of a base, such as potassium carbonate, at a temperature ranging between room and reflux temperature, preferably around 50° C. (step b). The hydroxy group ortho-positioned to the formyl group in an aldehyde of formula 12 can be alkylated (step c) by treating said aldehyde of formula 12 with an R₉-transferring reagent, such as an alkylhalide, alkylmesylate or alkyltosylate, in solvents like N,N-dimethylformamide, acetone or butan-2-one, in the presence of a weak base like cesium or potassium carbonate, optionally in the presence of sodium or potassium iodide, at a temperature ranging from room temperature to 140° C., yielding a tri-substituted benzaldehyde of formula (VI), which after further chemical modifications at R₉, e.g. removal of any protecting group(s) (step d), gives a benzaldehyde of formula (VI′).

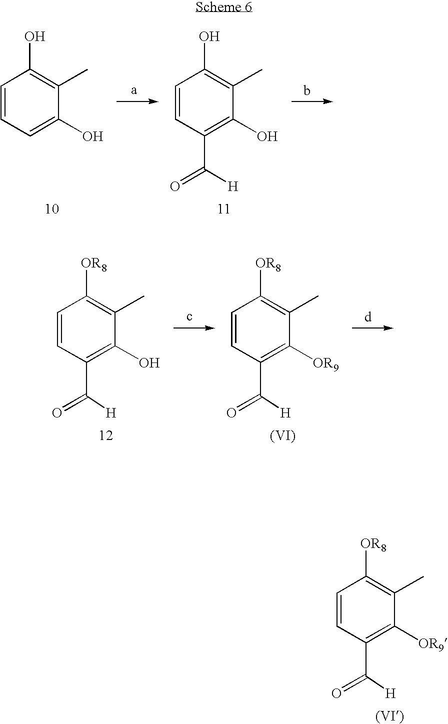

Particular embodiments of the invention are described in the following Examples, which serve to illustrate the invention in more detail without limiting its scope in any way.

### EXAMPLES

Temperatures are indicated in degrees Celsius (° C.). Unless otherwise indicated, the reactions take place at room temperature.

In mixtures, relations of parts of solvent or eluent or reagent mixtures in liquid form are given as volume relations (v/v), unless indicated otherwise.

Abbreviations and Acronyms Used:

AcOH: acetic acid, br: broad (spectral), BSA: bovine serum albumin, n-BuLi: n-butyllithium, CH₂Cl₂: dichloromethane, d: doublet (spectral), DIPEA: N,N-diisopropylethylamine, DMF: N,N-dimethylformamide, DMSO: dimethyl sulfoxide, EDTA: ethylenediaminetetraacetic acid, ESI: electrospray ionisation, Et₃N: triethylamine, EtOAc: ethyl acetate, EtOH: ethanol, g: gram, h: hour, HATU: O-(7-Azabenzotriazol-1-yl)-N,N,N′,N′-tetramethyluronium hexafluorophosphate, H₂O: water, HCl: hydrochloric acid, HPLC: high-performance liquid chromatography, k: kilo, K₂CO₃: potassium carbonate, l: liter, LiOH: lithium hydroxide, R: micro, m: milli, mol: mole, M: molar, MeOH: methanol, Me: methyl, min: minute, MS: mass spectrometry, N: normality of solution, NMR: nuclear magnetic resonance, NaCl: sodium chloride, NaHCO₃: sodium hydrogencarbonate, Na₂CO₃: sodium carbonate, NaOH: sodium hydroxyde, Na₂SO₄: sodium sulfate, 10% Pd/C: palladium, 10 weight % on activated carbon, Pd(PPh₃)₂Cl₂: dichlorobis(triphenylphosphine)palladium(II), ppm: part(s) per million, q: quartet (spectral), s: singlet (spectral), SDS: sodium dodecylsulfate, t: triplet (spectral), THF: tetrahydrofuran, TBAF: tetrabutylammonium fluoride, t_{R}: retention time.

Instruments and Analyses

HPLC/MS analyses were performed on a Waters Alliance HPLC, equipped with a Photodiode Array Detector Waters 996 and a Micromass ZQ™ Waters mass spectrometer (ESI)

Analytical HPLC conditions:

LC-A

Analytical HPLC on a Xterra™ MS C₁₈ column (2.1×50 mm, 5 μm, Waters).

Linear gradient of water/0.06% formic acid (A) and acetonitrile/0.06% formic acid (B) from 5% to 95% B over 6 min then final conditions held for 1 min; flow rate 0.25 ml/min.

LC-B

Analytical HPLC on a Xterra™ MS C₁₈ column (2.1×50 mm, 5 μm, Waters).

Linear gradient of water/0.06% formic acid (A) and acetonitrile/0.06% formic acid (B) from 5% to 95% B over 2 min then final conditions held for 0.5 min; flow rate 0.75 ml/min.

LC-C

Analytical HPLC on a Zorbax SB-Aq (4.6×50 mm, 5 μm, Agilent). Linear gradient of water/0.06% formic acid (A) and acetonitrile/0.06% formic acid (B) from 5% to 95% B over 1 min then final conditions held for 0.25 min; flow rate 3 ml/min.

Preparative HPLC conditions:

Xterra™ Prep MS C₁₈ column (19×50 mm, 5 μm, Waters).

¹H NMR spectra were recorded on a Varian Mercury 300VX FT-NMR spectrometer. Chemical shifts (δ) are reported in parts per million (ppm) downfield by reference to proton resonances resulting from incomplete deuteration of the NMR solvent, e.g. for dimetylsulfoxide δ(H) 2.49 ppm, for chloroform δ(H) 7.24 ppm.

### Example 1

### R₁ is Phenyl

### 4-(2,3-Dimethyl-4-propoxy-benzylidene)-1-phenyl-pyrazolidine-3,5-dione

A mixture of 1-phenyl-pyrazolidine-3,5-dione (53 mg, 0.30 mmol, prepared according to Conrad M. and Zart A., Ber. 1906, 2282-2288) and 2,3-dimethyl-4-propoxybenzaldehyde (Example 2b1b, 87 mg, 0.45 mmol), in absolute ethanol (4 ml) was heated at reflux for 16 h under inert atmosphere. After cooling to room temperature, the formed precipitate was collected by filtration. The solid was washed with absolute ethanol (3×2 ml) and dried in vacuo to give the title compound (80 mg, 76%) as a dark red solid: t_{R}=7.45 min (LC-A); MS (positive-ion mode): m/z 351.3 [M+H]⁺; MS (negative-ion mode): m/z 349.5 [M−H]⁻.

### Example 2

### R₁ is Phenyl

2a) The following products 2a1-2a65 were prepared by proceeding in a similar manner to the method described in Example 1, but using the respective aldehydes in place of 2,3-dimethyl-4-propoxybenzaldehyde:

2a1) 4-(4-Methoxy-3-methyl-benzylidene)-1-phenyl-pyrazolidine-3,5-dione, from 4-methoxy-3-methylbenzaldehyde (Fluka): t_{R}=6.48 min (LC-A); MS (pos.): m/z 309.3 [M+H]⁺; MS (neg.): m/z 307.5 [M−H]⁻.

2a2) 4-(4-Ethoxy-benzylidene)-1-phenyl-pyrazolidine-3,5-dione, from 4-ethoxybenzaldehyde (Aldrich): t_{R}=6.47 min (LC-A); MS (pos.): m/z 309.3 [M+H]⁺; MS (neg.): m/z 307.5 [M−H]⁻.

2a3) 4-(4-Ethoxy-3-methoxy-benzylidene)-1-phenyl-pyrazolidine-3,5-dione, from 4-ethoxy-3-methoxybenzaldehyde (Aldrich): t_{R}=6.16 min (LC-A); MS (pos.): [M+H]⁺ 339.3; MS (neg.): [M−H]⁻ 337.5.

2a4) 4-(2,4-Dimethoxy-benzylidene)-1-phenyl-pyrazolidine-3,5-dione, from 2,4-dimethoxybenzaldehyde (Aldrich): t_{R}=6.21 min (LC-A); MS (pos.): m/z 325.3 [M+H]⁺; MS (neg.): m/z 323.5 [M−H]⁻.

2a5) 4-Naphthalene-2-ylmethylene-1-phenyl-pyrazolidine-3,5-dione, from naphthalene-2-carbaldehyde (Aldrich): t_{R}=6.91 min (LC-A); MS (pos.): m/z 315.1 [M+H]⁺; MS (neg.): m/z 313.3 [M−H]⁻.

2a6) 4-(4-tert-Butyl-benzylidene)-1-phenyl-pyrazolidine-3,5-dione, from 4-tert-butyl-benzaldeyde (Fluka): t_{R}=7.35 min (LC-A); MS (pos.): m/z 321.4 [M+H]⁺; MS (neg.): m/z 319.6 [M−H]⁻.

2a7) 4-(2,3-Dimethyl-4-methoxy-benzylidene)-1-phenyl-pyrazolidine-3,5-dione, from 2,3-dimethyl-4-methoxybenzaldehyde (Aldrich): t_{R}=6.78 min (LC-A); MS (pos.): m/z 323.1 [M+H]⁺; MS (neg.): m/z 321.3 [M−H]⁻.

2a8) 4-(2,4-Dimethoxy-3-methyl-benzylidene)-1-phenyl-pyrazolidine-3,5-dione, from 2,4-dimethoxy-3-methylbenzaldeyde (Aldrich): t_{R}=6.67 min (LC-A); MS (pos.): m/z 339.1 [M+H]⁺; MS (neg.): m/z 337.3 [M−H]⁻.

2a9) 4-(3-Bromo-4-methoxy-benzylidene)-1-phenyl-pyrazolidine-3,5-dione, from 3-bromo-4-methoxybenzaldehyde (Acros): t_{R}=6.48 min (LC-A); MS (pos.): m/z 373.3, 375.2 [M+H]⁺; MS (neg.): m/z 371.3, 373.4 [M−H]⁻.

2a10) 1-Phenyl-4-(4-propoxy-benzylidene)-pyrazolidine-3,5-dione, from 4-propoxybenzaldehyde (Aldrich): t_{R}=6.86 min (LC-A); MS (pos.): m/z 323.3 [M+H]⁺; MS (neg.): m/z 321.5 [M−H]⁻.

2a11) 4-(4-Butoxy-benzylidene)-1-phenyl-pyrazolidine-3,5-dione, from 4-butoxybenzaldehyde (Acros): t_{R}=7.21 min (LC-A); MS (pos.): m/z 337.4 [M+H]⁺; MS (neg.): m/z 335.6 [M−H]⁻.

2a12) 4-(4-Ethoxy-3-methyl-benzylidene)-1-phenyl-pyrazolidine-3,5-dione, from 4-ethoxy-3-methylbenzaldehyde (Example 2b2): t_{R}=6.86 min (LC-A); MS (pos.): m/z 323.4 [M+H]⁺; MS (neg.): m/z 321.5 [M−H]⁻.

2a13) 4-(3-Methyl-4-propoxy-benzylidene)-1-phenyl-pyrazolidine-3,5-dione, from 3-methyl-4-propoxybenzaldehyde (Example 2b3): t_{R}==7.35 min (LC-A); MS (pos.): m/z 337.4 [M+H]⁺; MS (neg.): m/z 335.6 [M−H]⁻.

2a14) 4-(4-Butoxy-3-methyl-benzylidene)-1-phenyl-pyrazolidine-3,5-dione, from 4-butoxy-3-methylbenzaldehyde (Example 2b4): t_{R}=7.82 min (LC-A); MS (pos.): m/z 351.2 [M+H]⁺; MS (neg.): m/z 349.3 [M−H]⁻.

2a15) 4-(4-Hexyloxy-3-methyl-benzylidene)-1-phenyl-pyrazolidine-3,5-dione, from 4-hexyloxy-3-methylbenzaldehyde (Example 2b5): t_{R}=8.53 min (LC-A); MS (pos.): m/z 379.2 [M+H]⁺; MS (neg.): m/z 377.3 [M−H]⁻.

2a16) 4-(3-Methyl-4-pentyloxy-benzylidene)-1-phenyl-pyrazolidine-3,5-dione, from 3-methyl-4-pentyloxybenzaldehyde (Example 2b6): t_{R}=7.98 min (LC-A); MS (pos.): m/z 365.4 [M+H]⁺; MS (neg.): m/z 363.6 [M−H]⁻.

2a17) 4-(4-Cyclobutylmethoxy-3-methyl-benzylidene)-1-phenyl-pyrazolidine-3,5-dione, from 4-cyclobutylmethoxy-3-methylbenzaldehyde (Example 2b7): t_{R}=8.02 min (LC-A); MS (pos.): m/z 363.1 [M+H]⁺; MS (neg.): m/z 361.3 [M−H]⁻.

2a18) 4-[3-Methyl-4-(3-methyl-butoxy)-benzylidene]-1-phenyl-pyrazolidine-3,5-dione, from 3-methyl-4-(3-methylbutoxy)benzaldehyde (Example 2b8): t_{R}=8.14 min (LC-A); MS (pos.): m/z 365.1 [M+H]⁺; MS (neg.): m/z 363.3 [M−H]⁺.

2a19) 4-(4-iso-Butoxy-3-methyl-benzylidene)-1-phenyl-pyrazolidine-3,5-dione, from 4-iso-butoxy-3-methylbenzaldehyde (Example 2b9): MS (pos.): t_{R}=7.86 min (LC-A); m/z 351.1 [M+H]⁺; MS (neg.): m/z 349.3 [M−H]⁻.

2a20) 4-[4-(2-Methoxy-ethoxy)-3-methyl-benzylidene]-1-phenyl-pyrazolidine-3,5-dione, from 4-(2-methoxyethoxy)-3-methylbenzaldehyde (Example 2b10): t_{R}=6.31 min (LC-A); MS (pos.): m/z 353.4 [M+H]⁺; MS (neg.): m/z 351.5 [M−H]⁻.

2a21) 4-(3-Chloro-4-propoxy-benzylidene)-1-phenyl-pyrazolidine-3,5-dione, from 3-chloro-4-propoxybenzaldehyde (Example 2b11): t_{R}=7.18 min (LC-A); MS (pos.): m/z 357.3 [M+H]⁺; MS (neg.): m/z 355.5 [M−H]⁻.

2a22) 4-[4-(2-Hydroxy-ethoxy)-3-methyl-benzylidene]-1-phenyl-pyrazolidine-3,5-dione, from 4-(2-hydroxyethoxy)-3-methylbenzaldehyde (Example 2b12b): t_{R}=5.57 min (LC-A); MS (pos.): m/z 339.4 [M+H]⁺; MS (neg.): m/z 337.5 [M−H]⁻.

2a23) 4-(4-Cyclopropylmethoxy-3-methyl-benzylidene)-1-phenyl-pyrazolidine-3,5-dione, from 4-cyclopropylmethoxy-3-methyl-benzaldehyde (Example 2b13): t_{R}=7.39 min (LC-A); MS (pos.): m/z 349.1 [M+H]⁺; MS (neg.): m/z 347.3 [M−H]⁻.

2a24) 4-(4-Cyclopentyloxy-2,3-dimethyl-benzylidene)-1-phenyl-pyrazolidine-3,5-dione, from 4-cyclopentyloxy-2,3-dimethyl-benzaldehyde (Example 2b14): t_{R}=7.79 min (LC-A); MS (pos.): m/z 377.4 [M+H]⁺; MS (neg.): m/z 375.6 [M−H]⁻.

2a25) 1-Phenyl-4-(4-propoxy-5,6,7,8-tetrahydro-naphthalen-1-ylmethylene)-pyrazolidine-3,5-dione, from 4-propoxy-5,6,7,8-tetrahydro-naphthalene-1-carbaldehyde (Example 2b15c): t_{R}=7.97 min (LC-A); MS (pos.): m/z 377.2 [M+H]⁺; MS (neg.): m/z 375.4 [M−H]⁻.

2a26) 4-(2,3-Dimethyl-4-pent-1-ynyl-benzylidene)-1-phenyl-pyrazolidine-3,5-dione, from 2,3-dimethyl-4-pent-1-ynyl-benzaldehyde (Example 2b16b): t_{R}=7.77 min (LC-A); MS (pos.): m/z 359.2 [M+H]⁺; MS (neg.): m/z 357.3 [M−H]⁻.

2a27) 4-(2,3-Dimethyl-4-pentyl-benzylidene)-1-phenyl-pyrazolidine-3,5-dione, from 2,3-dimethyl-4-pentyl-benzaldehyde (Example 2b16c): t_{R}=8.10 min (LC-A); MS (pos.): m/z 363.3 [M+H]⁺; MS (neg.): m/z 361.4 [M−H]⁻.

2a28) 1-Phenyl-4-(4-propoxy-naphthalen-1-ylmethylene)-pyrazolidine-3,5-dione, from 4-propoxy-1-naphtaldehyde (Example 2b17): t_{R}=7.63 min (LC-A); MS (pos.): m/z 373.1 [M+H]⁺; MS (neg.): m/z 371.3 [M−H]⁻.

2a29) 1-Phenyl-4-[1-(7-propoxy-indan-4-yl)-methylidene]-pyrazolidine-3,5-dione, from 7-propoxy-indan-4-carbaldehyde (Example 2b18e): t_{R}=7.64 min (LC-A); MS (pos.): m/z 363.3 [M+H]⁺; MS (neg.): m/z 361.4 [M−H]⁻.

2a30) 1-Phenyl-4-[1-(5-propoxy-isoquinolin-8-yl)-methylidene]-pyrazolidine-3,5-dione, from 5-propoxy-isoquinoline-8-carbaldehyde (Example 2b19c): t_{R}=6.03 min (LC-A); MS (pos.): m/z 374.3 [M+H]⁺; MS (neg.): m/z 372.4 [M−H]⁻.

2a31) 1-Phenyl-4-[1-(8-propoxy-isoquinolin-5-yl)-methylidene]-pyrazolidine-3,5-dione, from 8-propoxy-isoquinoline-5-carbaldehyde (Example 2b20d): t_{R}=5.61 min (LC-A); MS (pos.): m/z 374.2 [M+H]⁺; MS (neg.): m/z 372.2 [M−H]⁻.

2a32) 4-[1-(2-tert-Butoxycarbonyl-5-propoxy-1,2,3,4-tetrahydro-isoquinolin-8-yl)-methylidene]-1-phenyl-pyrazolidine-3,5-dione, from 2-tert-butyloxycarbonyl-8-formyl-5-propoxy-1,2,3,4-tetrahydroisoquinoline (Example 2b21d): t_{R}=2.69 min (LC-B); MS (neg.): m/z 476.5 [M−H]⁻.

2a33) 4-(2,3-Dimethyl-4-ethanesulfonyloxy-benzylidene)-1-phenyl-pyrazolidine-3,5-dione, from 2,3-dimethyl-4-ethanesulfonyloxybenzaldehyde (Example 2b22): t_{R}=6.47 min (LC-A); MS (pos.): m/z 401.3 [M+H]⁺; MS (neg.): m/z 399.2 [M−H]⁻.

2a34) 4-[1-(2,4-Dipropoxy-phenyl)-methylidene]-1-phenyl-pyrazolidine-3,5-dione, from 2,4-dipropoxy-benzaldehyde (Example 2b23): t_{R}=1.18 min (LC-C); MS (pos.): m/z 381.1 [M+H]⁺; MS (neg.): m/z 379.3 [M−H]⁻.

2a35) 4-[1-(2,6-Dipropoxy-pyridin-3-yl)-methylidene]-1-phenyl-pyrazolidine-3,5-dione, from 2,6-dipropoxy-pyridine-3-carbaldehyde (Example 2b24b): t_{R}=1.21 min (LC-C); MS (pos.): m/z 382.0 [M+H]⁺; MS (neg.): m/z 380.3 [M−H]⁻.

2a36) 4-[1-(2-Hydroxy-3-methyl-4-propoxy-phenyl)-methylidene]-1-phenyl-pyrazolidine-3,5-dione, from 2-hydroxy-3-methyl-4-propoxy-benzaldehyde (Example 2b25): t_{R}=7.09 min (LC-A); MS (pos.): m/z 353.3 [M+H]⁺; MS (neg.): m/z 351.2 [M−H]⁻.

2a37) 4-[1-(2-Methoxy-3-methyl-4-propoxy-phenyl)-methylidene]-1-phenyl-pyrazolidine-3,5-dione, from 2-methoxy-3-methyl-4-propoxy-benzaldehyde (Example 2b26): t_{R}=7.39 min (LC-A); MS (pos.): m/z 367.3 [M+H]⁺; MS (neg.): m/z 365.4 [M−H]⁻.

2a38) 4-[1-(3-Methyl-2,4-dipropoxy-phenyl)-methylidene]-1-phenyl-pyrazolidine-3,5-dione, from 3-methyl-2,4-dipropoxy-benzaldehyde (Example 2b27): t_{R}=8.07 min (LC-A); MS (pos.): m/z 395.4 [M+H]⁺; MS (neg.): m/z 393.5 [M−H]⁻.

2a39) 4-[1-[2-(2-Methoxy-ethoxy)-3-methyl-4-propoxy-phenyl]-methylidene]-1-phenyl-pyrazolidine-3,5-dione, from 2-(2-methoxy-ethoxy)-3-methyl-4-propoxy-benzaldehyde (Example 2b28): t_{R}=7.32 min (LC-A); MS (pos.): m/z 411.3 [M+H]⁺; MS (neg.): m/z 409.3 [M−H]⁻.

2a40) 4-[1-[2-(2-Hydroxy-ethoxy)-3-methyl-4-propoxy-phenyl]-methylidene]-1-phenyl-pyrazolidine-3,5-dione, from 2-(2-hydroxy-ethoxy)-3-methyl-4-propoxy-benzaldehyde (Example 2b29b): t_{R}=6.67 min (LC-A); MS (pos.): m/z 397.4 [M+H]⁺; MS (neg.): m/z 395.3 [M−H]⁻.

2a41) 4-[1-[2-(3-Hydroxy-propoxy)-3-methyl-4-propoxy-phenyl]-methylidene]-1-phenyl-pyrazolidine-3,5-dione, from 2-(3-hydroxy-propoxy)-3-methyl-4-propoxy-benzaldehyde (Example 2b30b): t_{R}=6.96 min (LC-A); MS (pos.): m/z 411.2 [M+H]⁺; MS (neg.): m/z 409.2 [M−H]⁻.

2a42) 4-[1-[2-(4-Acetoxy-butoxy)-3-methyl-4-propoxy-phenyl]-methylidene]-1-phenyl-pyrazolidine-3,5-dione, from 2-(4-acetoxy-butoxy)-3-methyl-4-propoxy-benzaldehyde (Example 2b31): t_{R}=7.72 min (LC-A); MS (pos.): m/z 467.2 [M+H]⁺; MS (neg.): m/z 465.3 [M−H]⁻.

2a43) 4-[1-[2-(4-Hydroxy-butoxy)-3-methyl-4-propoxy-phenyl]-methylidene]-1-phenyl-pyrazolidine-3,5-dione, from 2-(4-hydroxy-butoxy)-3-methyl-4-propoxy-benzaldehyde (Example 2b32): t_{R}=6.85 min, 6.98 min (LC-A); MS (pos.): m/z 425.2 [M+H]⁺; MS (neg.): m/z 423.3 [M−H]⁻.

2a44) 4-[1-[2-(Ethoxycarbonyl-methoxy)-3-methyl-4-propoxy-phenyl]-methylidene]-1-phenyl-pyrazolidine-3,5-dione, from ethyl (6-formyl-2-methyl-3-propoxy-phenoxy)-acetate (Example 2b33): t_{R}=7.44 min (LC-A); MS (pos.): m/z 439.3 [M+H]⁺; MS (neg.): m/z 437.3 [M−H]⁻.

2a45) 4-[1-[2-(Carboxy-methoxy)-3-methyl-4-propoxy-phenyl]-methylidene]-1-phenyl-pyrazolidine-3,5-dione, from (6-formyl-2-methyl-3-propoxy-phenoxy)-acetic acid (Example 2b34): t_{R}=6.58 min (LC-A); MS (pos.): m/z 411.4 [M+H]⁺; MS (neg.): m/z 409.3 [M−H]⁻.

2a46) 4-[1-[2-(2-Amino-2-oxo-ethyloxy)-3-methyl-4-propoxy-phenyl]-methylidene]-1-phenyl-pyrazolidine-3,5-dione, from 2-(6-formyl-2-methyl-3-propoxy-phenoxy)-acetamide (Example 2b35): t_{R}=6.34 min (LC-A); MS (pos.): m/z 410.2 [M+H]⁺; MS (neg.): m/z 408.3 [M−H]⁻.

2a47) 4-[1-[2-(3-Ethoxycarbonyl-propoxy)-3-methyl-4-propoxy-phenyl]-methylidene]-1-phenyl-pyrazolidine-3,5-dione, from ethyl 4-(6-formyl-2-methyl-3-propoxy-phenoxy)-butanoate (Example 2b36): t_{R}=7.86 min (LC-A); MS (pos.): m/z 467.4 [M+H]⁺; MS (neg.): m/z 465.3 [M−H]⁻.

2a48) 4-[1-[2-(3-Carboxy-propoxy)-3-methyl-4-propoxy-phenyl]-methylidene]-1-phenyl-pyrazolidine-3,5-dione, from 4-(6-formyl-2-methyl-3-propoxy-phenoxy)-butanoic acid (Example 2b37): t_{R}=6.95 min (LC-A); MS (pos.): m/z 439.4 [M+H]⁺; MS (neg.): m/z 437.3 [M−H]⁻.

2a49) 4-[1-[2-(4-ethylamino-4-oxo-butoxy)-3-methyl-4-propoxy-phenyl]-methylidene]-1-phenyl-pyrazolidine-3,5-dione, from 4-(6-formyl-2-methyl-3-propoxy-phenoxy)-butanoic acid ethylamide (Example 2b38): t_{R}=7.16 min (LC-A); MS (pos.): m/z 466.2 [M+H]⁺; MS (neg.): m/z 464.3 [M−H]⁻.

2a50) 4-[1-[3-Methyl-2-(4-morpholin-4-yl-4-oxo-butoxy)-4-propoxy-phenyl]-methylidene]-1-phenyl-pyrazolidine-3,5-dione, from 3-methyl-2-(4-morpholin-4-yl-4-oxo-butoxy)-4-propoxy-benzaldehyde (Example 2b39): t_{R}=7.16 min (LC-A); MS (pos.): m/z 508.2 [M+H]⁺; MS (neg.): m/z 506.4 [M−H]⁻.

2a51) 4-[1-[2-(4-Ethoxycarbonyl-butoxy)-3-methyl-4-propoxy-phenyl]-methylidene]-1-phenyl-pyrazolidine-3,5-dione, from ethyl 5-(6-formyl-2-methyl-3-propoxy-phenoxy)-pentanoate (Example 2b40): t_{R}=8.37 min (LC-A); MS (pos.): m/z 481.2 [M+H]⁺; MS (neg.): m/z 479.4 [M−H]⁻.

2a52) 4-[1-[2-(4-Carboxy-butyloxy)-3-methyl-4-propoxy-phenyl]-methylidene]-1-phenyl-pyrazolidine-3,5-dione, from 5-(6-formyl-2-methyl-3-propoxy-phenoxy)-pentanoic acid (Example 2b41): t_{R}=7.34 min (LC-A); MS (pos.): m/z 453.2 [M+H]⁺; MS (neg.): m/z 451.3 [M−H]⁻.

2a53) 4-[1-[2-(5-Ethylamino-5-oxo-pentyloxy)-3-methyl-4-propoxy-phenyl]-methylidene]-1-phenyl-pyrazolidine-3,5-dione, from 5-(6-formyl-2-methyl-3-propoxy-phenoxy)-pentanoic acid ethylamide (Example 2b42): t_{R}=7.42 min (LC-A); MS (pos.): m/z 480.2 [M+H]⁺; MS (neg.): m/z 478.4 [M−H]⁻.

2a54) 4-[1-[3-Methyl-2-(5-morpholin-4-yl-5-oxo-pentyloxy)-4-propoxy-phenyl]-methylidene]-1-phenyl-pyrazolidine-3,5-dione, from 3-methyl-2-(5-morpholin-4-yl-5-oxo-pentyloxy)-4-propoxy-benzaldehyde (Example 2b43): t_{R}=7.29 min (LC-A); MS (pos.): m/z 522.3 [M+H]⁺; MS (neg.): m/z 520.4 [M−H]⁻.

2a55) 4-[1-[2-(2-Dimethylamino-ethoxy)-3-methyl-4-propoxy-phenyl]-methylidene]-1-phenyl-pyrazolidine-3,5-dione hydrochloride, from 2-(2-dimethylamino-ethoxy)-3-methyl-4-propoxy-benzaldehyde hydrochloride (Example 2b44): t_{R}=4.93 min (LC-A); MS (pos.): m/z 424.3 [M+H]⁺; MS (neg.): m/z 422.3 [M−H]⁻.

2a56) 4-[1-[3-Methyl-2-(2-morpholin-4-yl-ethoxy)-4-propoxy-phenyl]-methylidene]-1-phenyl-pyrazolidine-3,5-dione hydrochloride, from 3-methyl-2-(2-morpholin-4-yl-ethoxy)-4-propoxy-benzaldehyde hydrochloride (Example 2b45): t_{R}=5.05 min (LC-A); MS (pos.): m/z 466.3 [M+H]⁺; MS (neg.): m/z 464.3 [M−H]⁻.

2a57) 4-[1-[3-Methyl-2-(2-piperidin-1-yl-ethoxy)-4-propoxy-phenyl]-methylidene]-1-phenyl-pyrazolidine-3,5-dione hydrochloride, from 3-methyl-2-(2-piperidin-1-yl-ethoxy)-4-propoxy-benzaldehyde hydrochloride (Example 2b46): t_{R}=5.20 min (LC-A); MS (pos.): m/z 464.4 [M+H]⁺; MS (neg.): m/z 462.3 [M−H]⁻.

2a58) 4-[1-[2-(3-Dimethylamino-propoxy)-3-methyl-4-propoxy-phenyl]-methylidene]-1-phenyl-pyrazolidine-3,5-dione hydrochloride, from 2-(3-dimethylamino-propoxy)-3-methyl-4-propoxy-benzaldehyde hydrochloride (Example 2b47): t_{R}=5.09 min, 5.27 min (LC-A); MS (pos.): m/z 438.4 [M+H]⁺; MS (neg.): m/z 436.3 [M−H]⁻.

2a59) 4-[1-[2-(2,2-Dimethyl-[1,3]dioxolan-4-ylmethoxy)-3-methyl-4-propoxy-phenyl]-methylidene]-1-phenyl-pyrazolidine-3,5-dione, from 2-(2,2-dimethyl-[1,3]dioxolan-4-ylmethoxy)-3-methyl-4-propoxy-benzaldehyde (Example 2b48): t_{R}=7.68 min (LC-A); MS (pos.): m/z 467.2 [M+H]⁺; MS (neg.): m/z 465.3 [M−H]⁻.

2a60) 4-[1-[2-((4R)-2,2-Dimethyl-[1,3]dioxolan-4-ylmethoxy)-3-methyl-4-propoxy-phenyl]-methylidene]-1-phenyl-pyrazolidine-3,5-dione, from 2-((4R)-2,2-dimethyl-[1,3]dioxolan-4-ylmethoxy)-3-methyl-4-propoxy-benzaldehyde (Example 2b49): t_{R}=7.65 min (LC-A); MS (pos.): m/z 467.3 [M+H]⁺; MS (neg.): m/z 465.3 [M−H]⁻.

2a61) 4-[1-[2-((4S)-2,2-Dimethyl-[1,3]dioxolan-4-ylmethoxy)-3-methyl-4-propoxy-phenyl]-methylidene]-1-phenyl-pyrazolidine-3,5-dione, from 2-((4S)-2,2-dimethyl-[1,3]dioxolan-4-ylmethoxy)-3-methyl-4-propoxy-benzaldehyde (Example 2b50): t_{R}=7.66 min (LC-A); MS (pos.): m/z 467.3 [M+H]⁺; MS (neg.): m/z 465.3 [M−H]⁻.

2a62) 4-[1-[2-((2R)-3-Ethoxycarbonyl-2-hydroxy-propoxy)-3-methyl-4-propoxy-phenyl]-methylidene]-1-phenyl-pyrazolidine-3,5-dione, from ethyl (3R)-4-(6-formyl-2-methyl-3-propoxy-phenoxy)-3-hydroxy-butanoate (Example 2b51b): t_{R}=6.97 min, 7.11 min (LC-A); MS (pos.): m/z 483.3 [M+H]⁺; MS (neg.): m/z 481.3 [M−H]⁻.

2a63) 4-[1-[2-((2R)-3-Carboxy-2-hydroxy-propoxy)-3-methyl-4-propoxy-phenyl]-methylidene]-1-phenyl-pyrazolidine-3,5-dione, from (3R)-4-(6-formyl-2-methyl-3-propoxy-phenoxy)-3-hydroxy-butanoic acid (Example 2b52): t_{R}=6.18 min, 6.41 min (LC-A); MS (pos.): m/z 455.2 [M+H]⁺; MS (neg.): m/z 453.3 [M−H]⁻.

2a64) 4-[1-[2-((2S)-3-Carboxy-2-hydroxy-propoxy)-3-methyl-4-propoxy-phenyl]-methylidene]-1-phenyl-pyrazolidine-3,5-dione, from (3S)-4-(6-formyl-2-methyl-3-propoxy-phenoxy)-3-hydroxy-butanoic acid (Example 2b54): t_{R}=6.15 min, 6.39 min (LC-A); MS (pos.): m/z 455.1 [M+H]⁺; MS (neg.): m/z 453.2 [M−H]⁻.

2a65) 4-[1-[2-((4R,5S)-4-carboxy-2,2-dimethyl-[1,3]dioxolan-5-ylmethoxy)-3-methyl-4-propoxy-phenyl]-methylidene]-1-phenyl-pyrazolidine-3,5-dione, from (4R,5S)-5-(6-formyl-2-methyl-3-propoxy-phenoxymethyl)-2,2-dimethyl-[1,3]dioxolane-4-carboxylic acid (Example 2b56): t_{R}=6.92 min, 7.09 min (LC-A); MS (pos.): m/z 511.3 [M+H]⁺; MS (neg.): m/z 509.3 [M−H]⁻.

### 2a66) 1-Phenyl-4-[1-(5-propoxy-1,2,3,4-tetrahydro-isoquinolin-8-yl)-methylidene]-pyrazolidine-3,5-dione formate

To a mixture of 4-[1-(2-tert-butoxycarbonyl-5-propoxy-1,2,3,4-tetrahydro-isoquinolin-8-yl)-methylidene]-1-phenyl-pyrazolidine-3,5-dione (Example 2a32, 30 mg, 0.063 mmol), in THF (5 ml) was added a 2N solution of HCl in diethyl ether (628 μl, 1.25 mmol). After 1 h at room temperature, the volatiles were evaporated in vacuo and the resulting residue was purified by preparative HPLC to yield the title compound (2 mg, 8%) as an off-white solid: t_{R}=1.68 min (LC-B); MS (pos.): m/z 378.3 [M+H]⁺; MS (neg.): m/z 376.3 [M−H]⁻.

### 2a67) 4-[1-[2-(2,3-Dihydroxy-propoxy)-3-methyl-4-propoxy-phenyl]-methylidene]-1-phenyl-pyrazolidine-3,5-dione

To a mixture of 25% aqueous HCl (280 μl, 2.25 mmol) and EtOH (6 ml) was added 4-[1-[2-(2,2-dimethyl-[1,3]dioxolan-4-ylmethoxy)-3-methyl-4-propoxy-phenyl]-methylidene]-1-phenyl-pyrazolidine-3,5-dione (Example 2a59, 210 mg, 0.45 mmol). The reaction mixture was heated at 80° C. for 1 h. After cooling to room temperature, the formed precipitate was collected by filtration. The solid was washed with EtOH (2×3 ml) and dried in vacuo to give the title compound (98 mg, 51%) as an orange solid: t_{R}=6.04 min, 6.26 min (LC-A); MS (pos.): m/z 427.2 [M+H]⁺; MS (neg.): m/z 425.2 [M−H]⁻.

### 2a68) 4-[1-[2-((2S)-2,3-Dihydroxy-propoxy)-3-methyl-4-propoxy-phenyl]-methylidene]-1-phenyl-pyrazolidine-3,5-dione

Proceeding in a similar manner to the method described in Example 2a67, but using 4-[1-[2-((4R)-2,2-dimethyl-[1,3]dioxolan-4-ylmethoxy)-3-methyl-4-propoxy-phenyl]-methylidene]-1-phenyl-pyrazolidine-3,5-dione (Example 2a60) in place of 4-[1-[2-(2,2-dimethyl-[1,3]dioxolan-4-ylmethoxy)-3-methyl-4-propoxy-phenyl]-methylidene]-1-phenyl-pyrazolidine-3,5-dione gave the title compound: t_{R}=6.05 min, 6.27 min (LC-A); MS (pos.): m/z 427.2 [M+H]⁺; MS (neg.): m/z 425.2 [M−H]⁻.

### 2a69) 4-[1-[2-((2R)-2,3-Dihydroxy-propoxy)-3-methyl-4-propoxy-phenyl]-methylidene]-1-phenyl-pyrazolidine-3,5-dione

Proceeding in a similar manner to the method described in Example 2a67, but using 4-[1-[2-((4S)-2,2-dimethyl-[1,3]dioxolan-4-ylmethoxy)-3-methyl-4-propoxy-phenyl]-methylidene]-1-phenyl-pyrazolidine-3,5-dione (Example 2a61) in place of 4-[1-[2-(2,2-dimethyl-[1,3]dioxolan-4-ylmethoxy)-3-methyl-4-propoxy-phenyl]methylidene]-1-phenyl-pyrazolidine-3,5-dione gave the title compound: t_{R}=6.02 min, 6.24 min (LC-A), MS (pos.): m/z 427.3 [M+H]⁺; MS (neg.): m/z 425.2 [M−H]⁻.

### 2a70) 4-[1-[2-((2S,3R)-2,3-Dihydroxy-3-ethoxycarbonyl-propoxy)-3-methyl-4-propoxy-phenyl]-methylidene]-1-phenyl-pyrazolidine-3,5-dione

Proceeding in a similar manner to the method described in Example 2a67, but using 4-[1-[2-((4R,5S)-4-carboxy-2,2-dimethyl-[1,3]dioxolan-5-ylmethoxy)-3-methyl-4-propoxy-phenyl]-methylidene]-1-phenyl-pyrazolidine-3,5-dione (Example 2a65) in place of 4-[1-[2-(2,2-dimethyl-[1,3]dioxolan-4-ylmethoxy)-3-methyl-4-propoxy-phenyl]-methylidene]-1-phenyl-pyrazolidine-3,5-dione gave the title compound: t_{R}=6.72 min, 6.85 min (LC-A); MS (pos.): m/z 499.3 [M+H]⁺; MS (neg.): m/z 497.3 [M−H]⁻.

### 2a71) 4-[1-[2-((2S,3R)-3-carboxy-2,3-dihydroxy-propoxy)-3-methyl-4-propoxy-phenyl]-methylidene]-1-phenyl-pyrazolidine-3,5-dione

To a mixture of 1N aqueous HCl (160 μl, 0.16 mmol) and THF (1 ml) was added 4-[1-[2-((4R,5S)-4-carboxy-2,2-dimethyl-[1,3]dioxolan-5-ylmethoxy)-3-methyl-4-propoxy-phenyl]-methylidene]-1-phenyl-pyrazolidine-3,5-dione (Example 2a65, 20 mg, 0.04 mmol). The reaction mixture was heated at 70° C. for 16 h. After cooling to room temperature, the volatiles were evaporated in vacuo and the resulting residue was triturated with diisopropyl ether (4 ml). The suspended precipitate was collected by filtration and the solid washed with diisopropyl ether (2×4 ml) and dried in vacuo to give the title compound (12.5 mg, 80%) as a red solid: t_{R}=6.04 min, 6.31 min (LC-A), MS (pos.): m/z 471.2 [M+H]⁺; MS (neg.): m/z 469.3 [M−H]⁻.

2b) Aldehydes used in the Examples 1 and 2a12-2a65 were prepared as follows:

### 2b1) Preparation of 2,3-dimethyl-4-propoxybenzaldehyde

### 2b1a) 2,3-Dimethyl-4-hydroxybenzaldehyde

A solution of 2,3-dimethyl-4-methoxybenzaldehyde (6.40 g, 40 mmol) in anhydrous CH₂Cl₂ (160 ml) at −78° C. was treated with neat boron tribromide (7.56 ml, 80 mmol) dropwise via syringe. The solution was stirred at −78° C. for 10 min, warmed to room temperature and stirred for 48 h. The reaction mixture was cooled to 0° C. and quenched by the addition of water (100 ml). The separated aqueous phase was extracted with CH₂Cl₂ (3×100 ml). The combined organic phases were washed with brine (100 ml), dried over Na₂SO₄, filtered and concentrated. The resulting residue was purified by flash chromatography on silica gel using a gradient of EtOAc in heptane as eluent to yield the title compound (2.43 g, 40w) as a pale yellow solid: t_{R}=5.15 min (LC-A); MS (pos.): m/z 151.2 [M+H]⁺; MS (neg.): m/z 149.3 [M−H]⁻.

### 2b1b) 2,3-Dimethyl-4-propoxybenzaldehyde

To a solution of 2,3-dimethyl-4-hydroxy-benzaldehyde (Example 2b1a, 2.43 g, 16.2 mmol), in anhydrous DMF (33 ml) was added K₂CO₃ (2.24 g, 24.3 mmol). The mixture was stirred 10 min at room temperature and 1-bromopropane (1.47 ml, 17.8 mmol) was added. The mixture was heated at 50° C. overnight. After cooling to room temperature, the mixture was diluted with H₂O (150 ml) and extracted with EtOAc (3×100 ml). The combined organic phases were washed with H₂O (100 ml), brine (100 ml), dried over Na₂SO₄, filtered and concentrated. The resulting residue was purified by flash chromatography on silica gel using a gradient of EtOAc in heptane as eluent to yield the title compound (2.74 g, 88w) as a white solid: t_{R}=7.24 min (LC-A); MS (pos.): m/z 193.3 [M+H]⁺.

### 2b2) 4-Ethoxy-3-methylbenzaldehyde

Proceeding in a similar manner to the method described in Example 2b1b, but using 4-hydroxy-3-methylbenzaldehyde (Aldrich) in place of 2,3-dimethyl-4-hydroxy-benzaldehyde and bromoethane in place of 1-bromopropane, gave the title compound: t_{R}=6.36 min (LC-A); MS (pos.): m/z 165.2 [M+H]⁺.

### 2b3) 3-Methyl-4-propoxybenzaldehyde

Proceeding in a similar manner to the method described in Example 2b1b, but using 4-hydroxy-3-methylbenzaldehyde in place of 2,3-dimethyl-4-hydroxy-benzaldehyde, gave the title compound: t_{R}=6.89 min (LC-A); MS (pos.): m/z 179.3 [M+H]⁺.

### 2b4) 4-Butoxy-3-methylbenzaldehyde

Proceeding in a similar manner to the method described in Example 2b1b, but using 4-hydroxy-3-methylbenzaldehyde in place of 2,3-dimethyl-4-hydroxy-benzaldehyde, and 1-bromobutane in place of 1-bromopropane, gave the title compound: t_{R}=7.42 min (LC-A); MS (pos.): m/z 193.2 [M+H]⁺.

### 2b5) 4-Hexyloxy-3-methylbenzaldehyde

Proceeding in a similar manner to the method described in Example 2b1b, but using 4-hydroxy-3-methylbenzaldehyde in place of 2,3-dimethyl-4-hydroxy-benzaldehyde, and 1-bromohexane in place of 1-bromopropane, gave the title compound: t_{R}=8.25 min (LC-A); MS (pos.): m/z 221.3 [M+H]⁺.

### 2b6) 4-Pentyloxy-3-methylbenzaldehyde

Proceeding in a similar manner to the method described in Example 2b1b, but using 4-hydroxy-3-methylbenzaldehyde in place of 2,3-dimethyl-4-hydroxy-benzaldehyde, and 1-bromopentane in place of 1-bromopropane, gave the title compound: t_{R}=7.85 min (LC-A); MS (pos.): m/z 207.3 [M+H]⁺.

### 2b7) 4-Cyclobutylmethoxy-3-methylbenzaldehyde

Proceeding in a similar manner to the method described in Example 2b1b, but using 4-hydroxy-3-methylbenzaldehyde in place of 2,3-dimethyl-4-hydroxy-benzaldehyde, and bromomethylcyclobutane in place of 1-bromopropane, gave the title compound: t_{R}=7.57 min (LC-A); MS (pos.): m/z 205.2 [M+H]⁺.

### 2b8) 3-Methyl-4-(3-methyl-butoxy)benzaldehyde

Proceeding in a similar manner to the method described in Example 2b1b, but using 4-hydroxy-3-methylbenzaldehyde in place of 2,3-dimethyl-4-hydroxy-benzaldehyde, and 1-bromo-3-methylbutane in place of 1-bromopropane, gave the title compound: t_{R}=7.73 min (LC-A); MS (pos.): m/z 207.3 [M+H]⁺.

### 2b9) 4-iso-Butoxy-3-methylbenzaldehyde

Proceeding in a similar manner to the method described in Example 2b1b, but using 4-hydroxy-3-methylbenzaldehyde in place of 2,3-dimethyl-4-hydroxy-benzaldehyde, and 1-bromo-2-methylpropane in place of 1-bromopropane, gave the title compound: t_{R}=7.38 min (LC-A); MS (pos.): m/z 193.2 [M+H]⁺.

### 2b10) 4-(2-Methoxy-ethoxy)-3-methylbenzaldehyde

Proceeding in a similar manner to the method described in Example 2b1b, but using 4-hydroxy-3-methylbenzaldehyde in place of 2,3-dimethyl-4-hydroxy-benzaldehyde, and 1-bromo-2-methoxy-ethane in place of 1-bromopropane, gave the title compound: t_{R}=5.71 min (LC-A); MS (pos.): m/z 195.2 [M+H]⁺.

### 2b11) 3-Chloro-4-propoxybenzaldehyde

Proceeding in a similar manner to the method described in Example 2b1b, but using 4-hydroxy-3-chlorobenzaldehyde (ABCR) in place of 2,3-dimethyl-4-hydroxy-benzaldehyde, gave the title compound: t_{R}=6.80 min (LC-A); MS (pos.): m/z 199.2 [M+H]⁺.

### 2b12) Preparation of 4-(2-hydroxyethoxy)-3-methylbenzaldehyde

### 2b12a) 4-[2-(tert-Butyl-dimethylsilanyloxy)-ethoxy]-3-methylbenzaldehyde

Proceeding in a similar manner to the method described in Example 2b1b, but using 4-hydroxy-3-methylbenzaldehyde in place of 2,3-dimethyl-4-hydroxy-benzaldehyde, and (2-bromethoxy)-tert-butyl-dimethylsilane in place of 1-bromopropane, gave the title compound: t_{R}=8.39 min (LC-A); MS (pos.): m/z 295.3 [M+H]⁺.

### 2b12b) 4-(2-Hydroxyethoxy)-3-methylbenzaldehyde

To a solution of 4-[2-(tert-butyl-dimethylsilanyloxy)-ethoxy]-3-methylbenzaldehyde (Example 2b12a, 500 mg, 1.70 mmol), and AcOH (60 μl, 1.0 mmol) in THF (5 ml) was added dropwise a 1N solution of TBAF (2 ml, 2 mmol) in THF. The reaction mixture was stirred overnight at room temperature. The mixture was then diluted with H₂O (20 ml) and extracted with EtOAc (3×20 ml). The combined organic phases were washed with H₂O (20 ml), brine (20 ml), dried over Na₂SO₄, filtered and concentrated. The resulting residue was purified by flash chromatography on silica gel using a gradient of EtOAc in heptane as eluent to yield the title compound as colorless oil (259 mg, 85%): t_{R}=4.68 min (LC-A); MS (pos.): m/z 181.2 [M+H]⁺.

### 2b13) 4-Cyclopropylmethoxy-3-methyl-benzaldehyde

Proceeding in a similar manner to the method described in Example 2b1b, but using 4-hydroxy-3-methylbenzaldehyde in place of 2,3-dimethyl-4-hydroxy-benzaldehyde, and (bromomethyl)cyclopropane in place of 1-bromopropane, gave the title compound: t_{R}=6.89 min (LC-A); MS (pos.): m/z 191.2 [M+H]⁺.

### 2b14) 4-Cyclopentyloxy-2,3-dimethyl-benzaldehyde

Proceeding in a similar manner to the method described in Example 2b1b, but using cyclopentyl bromide in place of 1-bromopropane, gave the title compound: t_{R}=7.68 min (LC-A); MS (pos.): m/z 219.6 [M+H]⁺.

### 2b15) Preparation of 4-propoxy-5,6,7,8-tetrahydro-naphthalene-1-carbaldehyde

### 2b15a) 5-Propoxy-1,2,3,4-tetrahydro-naphthalene

Proceeding in a similar manner to the method described in Example 2b1b, but using 5,6,7,8-tetrahydronaphtalen-1-ol (Acros) in place of 2,3-dimethyl-4-hydroxy-benzaldehyde, gave the title compound: t_{R}=8.40 min (LC-A); MS (pos.): m/z 191.4 [M+H]⁺.

### 2b15b) 5-Bromo-8-propoxy-1,2,3,4-tetrahydro-naphthalene

To a stirred solution of 5-propoxy-1,2,3,4-tetrahydro-naphthalene (Example 2b15a, 1.14 g, 6 mmol), in acetonitrile (30 ml) was added N-bromosuccinimide (1.17 g, 6.6 mmol). The reaction mixture was stirred at room temperature for 2 h. The solvent was then evaporated under reduced pressure and water (20 ml) was added to the resulting residue. The aqueous solution was then extracted with EtOAc (3×25 ml). The combined organic phases were washed with H₂O (20 ml), brine (20 ml), dried over Na₂SO₄, filtered and concentrated. The resulting residue was purified by flash chromatography on silica gel using a gradient of EtOAc in heptane as eluent to yield the title compound as colorless oil (1.60 g, 99%): t_{R}=9.02 min (LC-A).

### 2b15c) 4-Propoxy-5,6,7,8-tetrahydro-naphthalene-1-carbaldehyde

To solution of 5-bromo-8-propoxy-1,2,3,4-tetrahydro-naphthalene (Example 2b15b, 1.60 g, 5.9 mmol), in THF (12.9 ml) at −78° C. was added dropwise in 5 min a 1.6N solution of n-BuLi in hexanes (4.4 ml, 7.1 mmol). The reaction mixture was stirred at −78° C. for 5 min and DMF (2.5 ml, 32.2 mmol) was then added. After warming to room temperature in 30 min and stirring at this temperature for 30 min, the reaction mixture was diluted with water (20 ml) and extracted with EtOAc (3×50 ml). The combined organic phases were washed with brine (50 ml), dried over Na₂SO₄, filtered and concentrated. The resulting residue was purified by flash chromatography on silica gel using a gradient of EtOAc in heptane as eluent to yield the title compound as pale yellow oil (800 mg, 57%): t_{R}=7.81 min (LC-A); MS (pos.): m/z 219.1 [M+H]⁺.

### 2b16) Preparation of 2,3-diethyl-4-pentyl-benzaldehyde

### 2b16a) 2,3-Dimethyl-4-trifluoromethanesulfonyloxybenzaldehyde

A mixture of 2,3-dimethyl-4-hydroxybenzaldehyde (Example 2b1a, 1.0 g, 6.7 mmol), N-phenylbis(trifluoromethanesulphonimide) (2.38 g, 6.7 mmol) and DIPEA (1.14 ml, 6.7 mmol) in CH₂Cl₂ (10 ml) was stirred overnight at room temperature. The reaction mixture was diluted with CH₂Cl₂ (50 ml). The resulting organic phase was washed consecutively with saturated aqueous NaHCO₃ (2×30 ml) and brine (30 ml), dried over MgSO₄, filtered and concentrated. The resulting residue was purified by flash chromatography on silica gel using a gradient of EtOAc in heptane as eluent to yield the title compound as colorless oil (1.50 g, 80w): t_{R}=7.09 min (LC-A); MS (pos.): m/z 283.0 [M+H]⁺.

### 2b16b) 2,3-Dimethyl-4-pent-1-ynyl-benzaldehyde

To a degassed solution containing Pd(PPh₃)₂Cl₂ (362 mg, 0.52 mmol), copper iodide (98 mg, 0.52 mmol), 2,3-dimethyl-4-trifluoromethanesulfonyloxybenzaldehyde (Example 2b16a, 1.47 g, 5.2 mmol), and DIPEA (2.83 ml, 16.5 mmol) in DMF (5 ml), was added 1-pentyne (1.02 ml, 10.3 mmol). After 24 h at room temperature, the reaction mixture was poured into water (50 ml) and extracted with EtOAc (2×50 ml). The combined organic phases were washed with brine (50 ml), dried over MgSO₄, filtered and concentrated. The resulting residue was purified by flash chromatography on silica gel using a gradient of EtOAc in heptane as eluent to yield the title compound as pale yellow oil (971 mg, 94%): t_{R}=7.78 min (LC-A); MS (pos.): m/z 201.4 [M+H]⁺.

### 2b16c) 2,3-Dimethyl-4-pentyl-benzaldehyde

A mixture of 2,3-dimethyl-4-pent-1-ynyl-benzaldehyde (Example 2b16b, 300 mg, 1.50 mmol), and 10% Pd/C (45 mg) in EtOAc (4 ml) was stirred for 1 h 30 under hydrogen at room temperature. After filtration on a Celite pad, which was washed with EtOAc, the filtrate was concentrated in vacuo to yield the title compound as colorless oil (285 mg, 93%): t_{R}=8.12 min (LC-A); MS (pos.): m/z 205.4 [M+H]⁺.

### 2b17) 4-Propoxy-1-naphtaldehyde

Proceeding in a similar manner to the method described in Example 2b1b, but using 4-hydroxy-1-naphtaldehyde (Aldrich) in place of 2,3-dimethyl-4-hydroxy-benzaldehyde, gave the title compound: t_{R}=7.38 min (LC-A); MS (pos.): m/z 215.3 [M+H]⁺.

### 2b18) Preparation of 7-Propoxy-indan-4-carbaldehyde

### 2b18a) 4-Propoxy-indan-1-one

Proceeding in a similar manner to the method described in Example 2b1b, but using 4-hydroxy-indan-1-one (TCI) in place of 2,3-dimethyl-4-hydroxy-benzaldehyde, gave the title compound: R=6.50 min (LC-A); MS (pos.): m/z 191.4 [M+H]⁺.

### 2b18b) 4-Propoxy-indan-1-ol

Sodium borohydride (275 mg, 7.3 mmol) was added in small portions to a solution of 4-propoxy-indan-1-one (Example 2b18a, 2.77 g, 14.6 mmol), in MeOH (10 ml) at 0° C. The solution was stirred at 0° C. for 10 min, allowed to warm to room temperature. After 48 h at room temperature, the excess of sodium borohydride was destroyed by addition of acetic acid (2.5 ml). The reaction mixture was then diluted with water (100 ml) and extracted with tert-butyl methyl ether (2×50 ml). The combined organic phases were washed with H₂O (50 ml), brine (50 ml), dried over Na₂SO₄, filtered and concentrated to yield the title compound (1.80 g, 64%) as a pale yellow solid: t_{R}=6.00 min (LC-A).

### 2b18c) 4-Propoxy-indane

A mixture of 4-propoxy-indan-1-ol (Example 2b18b, 2.35 g, 12.2 mmol), and 10% Pd/C (235 mg) in an ethanolic 0.5N HCl solution (50 ml) was stirred for 1 h under hydrogen at room temperature. After filtration on a Celite pad, which was washed with EtOAc, the filtrate was concentrated in vacuo to yield the title compound as a pale brown oil (1.81 g, 84w): t_{R}=8.01 min (LC-A); MS (pos.): m/z 177.3 [M+H]⁺.

### 2b18d) 4-Bromo-7-propoxy-indane

Proceeding in a similar manner to the method described in Example 2b15b, but using 4-propoxy-indane (Example 2b18c) in place of 5-propoxy-1,2,3,4-tetrahydro-naphtalene, gave the title compound: t_{R}=8.59 min (LC-A).

### 2b18e) 7-Propoxy-indan-4-carbaldehyde

Proceeding in a similar manner to the method described in Example 2b15c, but using 4-bromo-7-propoxy-indane (Example 2b18d) in place of 5-bromo-8-propoxy-1,2,3,4-tetrahydro-naphtalene, gave the title compound: t_{R}=7.40 min (LC-A); MS (pos.): m/z 205.3 [M+H]⁺.

### 2b19) 5-Propoxy-isoquinoline-8-carbaldehyde

### 2b19a) 5-Propoxy-isoquinoline

Sodium hydride (779 mg, 32.4 mmol) was added in small portions to a solution of 5-hydroxy-isoquinoline (3.0 g, 20.6 mmol, Aldrich) in anhydrous DMF (45 ml) at 0° C. The mixture was stirred 5 min at 0° C. and 1-bromopropane (2.10 ml, 20.6 mmol) was added. The mixture was then allowed to warm to room temperature. After 14 h at room temperature, the mixture was diluted with H₂O (50 ml) and extracted with EtOAc (2×50 ml). The combined organic phases were washed with 1N aqueous NaOH (2×50 ml), brine (50 ml), dried over Na₂SO₄, filtered and concentrated. The resulting residue was purified by flash chromatography on silica gel using a gradient of EtOAc in heptane to yield the title compound (3.23 g, 83%) as a violet oil: t_{R}=1.63 min (LC-B); MS (pos.): m/z 188.3 [M+H]⁺.

### 2b19b) 8-Bromo-5-propoxy-isoquinoline

Proceeding in a similar manner to the method described in Example 2b15b, but using 5-propoxy-isoquinoline (Example 2b19a) in place of 5-propoxy-1,2,3,4-tetrahydro-naphtalene, gave the title compound: t_{R}=2.37 min (LC-B); MS (pos.): m/z 266.2, 268.2 [M+H]⁺.

### 2b19c) 5-Propoxy-isoquinoline-8-carbaldehyde

To solution of 8-bromo-5-propoxy-isoquinoline (Example 2b19b, 174 mg, 0.65 mol)₁ in THF (5 ml) at −78° C. was added dropwise in 5 min a 2.5N solution of n-BuLi in hexanes (262 μl, 0.65 mmol). The reaction mixture was stirred at −78° C. for 5 min and anhydrous methyl formate (65 μl, 1.05 mmol) was then added. After stirring at −78° C. for 20 min, saturated aqueous NH₄Cl was added and the reaction mixture was allowed to warm to room temperature. The reaction mixture was then diluted with water (50 ml) and extracted with EtOAc (2×50 ml). The combined organic phases were washed with water (50 ml), brine (50 ml), dried over Na₂SO₄, filtered and concentrated. The resulting residue was purified by flash chromatography on silica gel using a gradient of EtOAc in heptane as eluent to yield the title compound as brown solid (50 mg, 36%): t_{R}=1.67 min (LC-B); MS (pos.): m/z 216.3 [M+H]⁺.

### 2b20) Preparation of 8-propoxy-isoquinoline-5-carbaldehyde

### 2b20a) 2-Propoxy-benzaldehyde

Proceeding in a similar manner to the method described in Example 2b1b, but using 2-hydroxybenzaldehyde (Acros) in place of 2,3-dimethyl-4-hydroxy-benzaldehyde, gave the title compound: t_{R}=6.59 min (LC-A); MS (pos.): m/z 165.3 [M+H]⁺.

### 2b20b) 8-Propoxy-isoquinoline

Proceeding in a similar manner to the method described by Hendrickson J. B. and Rodriguez C., J. Org. Chem. 1983, 3346-3347, but using 2-propoxy-benzaldehyde (Example 2b20a) as starting material, gave the title compound: t_{R}=1.44 min (LC-B); MS (pos.): m/z 188.4 [M+H]⁺.

### 2b20c) 5-Bromo-8-propoxy-isoquinoline

Proceeding in a similar manner to the method described in Example 2b15b, but using 8-propoxy-isoquinoline (Example 2b20b) in place of 5-propoxy-1,2,3,4-tetrahydro-naphtalene, gave the title compound: t_{R}=2.11 min (LC-B); MS (pos.): m/z 266.2, 268.2 [M+H]⁺.

### 2b20d) 8-Propoxy-isoquinoline-5-carbaldehyde

Proceeding in a similar manner to the method described in Example 2b19c, but using 5-bromo-8-propoxy-isoquinoline (Example 2b20c) in place of 8-bromo-5-propoxy-isoquinoline, gave the title compound: t_{R}=1.67 min (LC-B); MS (pos.): m/z 216.6 [M+H]⁺.

### 2b21) Preparation of 2-tert-butyloxycarbonyl-8-formyl-5-propoxy-1,2,3,4-tetrahydroisoquinoline

### 2b21a) 5-Propoxy-1,2,3,4-tetrahydro-isoquinoline hydrochloride

A mixture of 5-propoxy-isoquinoline (Example 2b19a, 2.0 g, 10.7 mmol), concentrated aqueous HCl (1.5 ml) and platinum oxide in ethanol was hydrogenated overnight at room temperature. After filtration on a Celite pad, which was washed with ethanol, the filtrate was concentrated in vacuo to yield the title compound as an off-white solid (2.32 g, 95%): t_{R}=0.37 min, 1.46 min (LC-B); MS (pos.): m/z 192.3 [M+H]⁺.

### 2b21b) 2-tert-Butyloxycarbonyl-5-propoxy-1,2,3,4-tetrahydro-isoquinoline

A mixture of 5-propoxy-1,2,3,4-tetrahydro-isoquinoline hydrochloride (Example 2b21a, 1.16 g, 5.1 mmol), Et₃N (0.71 ml, 5.1 mmol), di-tert-butyl dicarbonate (1.12 g, 5.40 mmol) in 1,4-dioxane-H₂O (12.5 ml-12.5 ml) was stirred for 18 h at room temperature, then concentrated in vacuo. The residue was dissolved in EtOAc (100 ml), washed with H₂O (50 ml) and brine (50 ml). The organic phase was dried over Na₂SO₄, filtered and concentrated. The resulting residue was purified by flash chromatography on silica gel using a gradient of EtOAc in heptane as eluent to yield the title compound as a white solid (1.05 g, 75%): t_{R}=2.75 min (LC-B); MS (pos.): m/z 292.4 [M+H]⁺.

### 2b21c) 8-Bromo-2-tert-butyloxycarbonyl-5-propoxy-1,2,3,4-tetrahydroisoquinoline

Proceeding in a similar manner to the method described in Example 2b15b, but using 2-tert-butyloxycarbonyl-5-propoxy-1,2,3,4-tetrahydro-isoquinoline (Example 2b21b) in place of 5-propoxy-1,2,3,4-tetrahydro-naphtalene, gave the title compound: t_{R}=2.97 min (LC-B).

### 2b21d) 2-tert-Butyloxycarbonyl-8-formyl-5-propoxy-1,2,3,4-tetrahydroisoquinoline

Proceeding in a similar manner to the method described in Example 2b19c, but using 8-bromo-2-tert-butyloxycarbonyl-5-propoxy-1,2,3,4-tetrahydroisoquinoline (Example 2b21c) in place of 8-bromo-5-propoxy-isoquinoline, gave the title compound: t_{R}=2.63 min (LC-B); MS (pos.): m/z 320.3 [M+H]⁺.

### 2b22) 2,3-Dimethyl-4-ethanesulfonyloxybenzaldehyde

To a solution of 2,3-dimethyl-4-hydroxybenzaldehyde (Example 2b1b, 1 g, 6.7 mmol), and Et₃N (4.5 ml, 32.6 mmol) in CH₂Cl₂ (30 ml) at 0° C. was added dropwise ethansulfonyl chloride. The reaction mixture was allowed to warm to room temperature. After 2 h, the reaction mixture was diluted with CH₂Cl₂ (50 ml). The resulting organic phase was washed consecutively with 1N aqueous NaOH (30 ml), water (2×30 ml), dried over MgSO₄, filtered and concentrated. The resulting residue was purified by flash chromatography on silica gel using a gradient of EtOAc in heptane as eluent to yield the title compound as colorless oil (1.50 g, 80%): t_{R}=6.04 min (LC-A); MS (pos.): m/z 243.3 [M+H]⁺.

### 2b23) 2,4-Dipropoxy-benzaldehyde

To a solution of 2,4-dihydroxy-benzaldehyde (300 mg, 2.2 mmol, Acros) in anhydrous DMF (2 ml) was added K₂CO₃ (913 mg, 6.6 mmol). The mixture was stirred 10 min at room temperature and 1-bromopropane (454 μl, 5 mmol) was added. The mixture was heated at 60° C. overnight. After cooling to room temperature, the mixture was diluted with H₂O (10 ml) and extracted with EtOAc (3×10 ml). The combined organic phases were washed with H₂O (2×20 ml), brine (20 ml), dried over Na₂SO₄, filtered and concentrated. The resulting residue was purified by flash chromatography on silica gel using a gradient of EtOAc in heptane as eluent to yield the title compound (412 mg, 84%) as a pale yellow solid: t_{R}=1.13 min (LC-C); MS (pos.): m/z 223.1 [M+H]⁺.

### 2b24) Preparation of 2,6-dipropoxy-pyridine-3-carbaldehyde

### 2b24a) 2,6-Dipropoxy-pyridine

Sodium hydride (1.08 g, 27.0 mmol) was added portionwise to a solution of n-propanol (2.03 ml, 27.0 mmol) in anhydrous DMF (8 ml) at 0° C. The reaction mixture was then allowed to warm to room temperature. After 30 min at this temperature, a solution of 2,6-dichloropyridine (1 g, 6.8 mmol) in DMF (5 ml) was added and the reaction mixture was heated at 100° C. for 1 h 30. After cooling and dilution with H₂O (50 ml), the reaction mixture was extracted with EtOAc (3×50 ml). The combined organic phases were washed with H₂O (2×50 ml), brine (2×20 ml), dried over Na₂SO₄, filtered and concentrated. The resulting residue was purified by flash chromatography on silica gel using a gradient of EtOAc in heptane as eluent to yield the title compound as a pale brown oil: t_{R}=1.15 min (LC-C); MS (pos.): m/z 196.6 [M+H]⁺, used in the next step without further purification.

### 2b24b) 2,6-Dipropoxy-pyridine-3-carbaldehyde

To anhydrous DMF (1.03 ml, 13.3 mmol) at 0° C. was added dropwise phosphorous oxychloride (1.22 ml, 13.3 mmol). The reaction mixture was then allowed to warm to room temperature. After 30 min, a solution of 2,6-dipropoxy-pyridine (Example 2b24a) in DMF (3 ml) was added dropwise. The reaction mixture was then heated at 70° C. for 1 h 30, allowed to stir at room temperature overnight and then poured in a 2N aqueous NaOH solution (25 ml). The aqueous solution was extracted with EtOAc (50 ml). The resulting aqueous phase was washed with H₂O (2×25 ml), brine (2×25 ml), dried over Na₂SO₄, filtered and concentrated. The resulting residue was purified by flash chromatography on silica gel using a gradient of EtOAc in heptane as eluent to yield the title compound (230 mg, 15%) as a yellow oil: t_{R}=1.17 min (LC-C); MS (pos.): m/z 224.0 [M+H]⁺.

### 2b25) 2-Hydroxy-3-methyl-4-propoxy-benzaldehyde

To a solution of 3-methyl-2,4-dihydroxybenzaldehyde (9.2 g, 60.4 mmol), prepared according to Nielsen S. F. et al., J. Med. Chem. 1998, 4819-4832, in anhydrous acetonitrile (300 ml) was added K₂CO₃ (8.76 g, 63.5 mmol). The mixture was stirred 10 min at room temperature and 1-bromopropane (11.0 ml, 121 mmol) was added. The mixture was heated at 50° C. for 60 h. After cooling to room temperature, the volatiles were evaporated in vacuo. The resulting residue was dissolved with H₂O (200 ml) and the pH of the aqueous solution was adjusted to 1 by addition of 1N aqueous HCl. The mixture was then extracted with EtOAc (2×200 ml). The combined organic phases were washed with H₂O (2×50 ml), brine (2×50 ml), dried over Na₂SO₄, filtered and concentrated. The resulting residue was purified by flash chromatography on silica gel using a gradient of EtOAc in heptane as eluent to yield the title compound (8.34 g, 85%) as a pale yellow solid: t_{R}=7.26 min (LC-A); MS (pos.): m/z 195.3 [M+H]⁺; MS (neg.): m/z 193.2 [M−H]⁻.

### 2b26) 2-Methoxy-3-methyl-4-propoxy-benzaldehyde

To a solution of 2-hydroxy-3-methyl-4-propoxy-benzaldehyde (Example 2b25, 400 mg, 2.06 mmol), in anhydrous DMF (6 ml) was added K₂CO₃ (427 mg, 3.09 mmol). The mixture was stirred 10 min at room temperature and methyl iodide (193 μl, 3.09 mmol) was added. The mixture was stirred at room temperature for 3 h then diluted with H₂O (40 ml) and extracted with CH₂Cl₂ (3×20 ml). The combined organic phases were washed with H₂O (2×20 ml), brine (20 ml), dried over MgSO₄, filtered and concentrated. The resulting residue was purified by flash chromatography on silica gel using a gradient of EtOAc in heptane as eluent to yield the title compound (332 mg, 77%) as a pale yellow oil: t_{R}=6.92 min (LC-A); MS (pos.): m/z 209.4 [M+H]⁺.

### 2b27) 3-Methyl-2,4-dipropoxy-benzaldehyde

Proceeding in a similar manner to the method described in Example 2b23, but using 3-methyl-2,4-dihydroxybenzaldehyde, prepared according to Nielsen S. F. et al., J. Med. Chem. 1998, 4819-4832 in place of 2,4-dihydroxy-benzaldehyde gave the title compound: t_{R}=7.76 min (LC-A); MS (pos.): m/z 237.4 [M+H]⁺.

### 2b28) 2-(2-Methoxy-ethoxy)-3-methyl-4-propoxy-benzaldehyde

Proceeding in a similar manner to the method described in Example 2b1b, but using 2-hydroxy-3-methyl-4-propoxy-benzaldehyde (Example 2b25) in place of 2,3-dimethyl-4-hydroxy-benzaldehyde, and 1-bromo-2-methoxy-ethane in place of 1-bromopropane, gave the title compound: t_{R}=6.90 min (LC-A); MS (pos.): m/z 253.3 [M+H]⁺.

### 2b29) Preparation of 2-(2-Hydroxy-ethoxy)-3-methyl-4-propoxy-benzaldehyde

### 2b29a) 2-[2-(tert-Butyl-dimethyl-silanyloxy)-ethoxy]-3-methyl-4-propoxy-benzaldehyde

Proceeding in a similar manner to the method described in Example 2b1b, but using 2-hydroxy-3-methyl-4-propoxy-benzaldehyde (Example 2b25) in place of 2,3-dimethyl-4-hydroxy-benzaldehyde, and (2-bromethoxy)-tert-butyl-dimethylsilane in place of 1-bromopropane, gave the title compound: t_{R}=9.15 min (LC-A); MS (pos.): m/z 353.6 [M+H]⁺.

### 2b29b) 2-(2-Hydroxy-ethoxy)-3-methyl-4-propoxy-benzaldehyde

Proceeding in a similar manner to the method described in Example 2b12b, but using 2-[2-(tert-butyl-dimethyl-silanyloxy)-ethoxy]-3-methyl-4-propoxy-benzaldehyde (Example 2b29a) in place of 4-[2-(tert-butyl-dimethylsilanyloxy)-ethoxy]-3-methylbenzaldehyde gave the title compound: t_{R}=6.06 min (LC-A); MS (pos.): m/z 239.3 [M+H]⁺.

### 2b30) Preparation of 2-(3-hydroxy-propoxy)-3-methyl-4-propoxy-benzaldehyde

### 2b30a) 2-[3-(tert-Butyl-dimethyl-silanyloxy)-propoxy]-3-methyl-4-propoxy-benzaldehyde

Proceeding in a similar manner to the method described in Example 2b1b, but using 2-hydroxy-3-methyl-4-propoxy-benzaldehyde (Example 2b25) in place of 2,3-dimethyl-4-hydroxy-benzaldehyde, and (3-bromopropoxy)-tert-butyl-dimethylsilane in place of 1-bromopropane, gave the title compound: t_{R}=3.24 min (LC-B); MS (pos.): m/z 367.2 [M+H]⁺.

### 2b30b) 2-(3-Hydroxy-propoxy)-3-methyl-4-propoxy-benzaldehyde

Proceeding in a similar manner to the method described in Example 2b12b, but using 2-[3-(tert-butyl-dimethyl-silanyloxy)-propoxy]-3-methyl-4-propoxy-benzaldehyde (Example 2b30a) in place of 4-[2-(tert-butyl-dimethylsilanyloxy)-ethoxy]-3-methylbenzaldehyde gave the title compound: t_{R}=6.14 min (LC-A); MS (pos.): m/z 253.2 [M+H]⁺.

### 2b31) 2-(4-Acetoxy-butoxy)-3-methyl-4-propoxy-benzaldehyde

Proceeding in a similar manner to the method described in Example 2b1b, but using 2-hydroxy-3-methyl-4-propoxy-benzaldehyde (Example 2b25) in place of 2,3-dimethyl-4-hydroxy-benzaldehyde, and 4-bromobutyl acetate in place of 1-bromopropane, gave the title compound: t_{R}=2.46 min (LC-B); MS (pos.): m/z 331.3 [M+Na]⁺.

### 2b32) 2-(4-Hydroxy-butoxy)-3-methyl-4-propoxy-benzaldehyde

To a mixture of 2-(4-acetoxy-butoxy)-3-methyl-4-propoxy-benzaldehyde (Example 2b31, 200 mg, 0.65 mmol), in THF (2 ml) was added a 2N aqueous LiOH solution (648 μl, 1.3 mmol). The reaction mixture was stirred at room temperature for 12 h, heated at 60° C. for 3 h and then cooled to room temperature. After acidification by addition of 1N HCl aqueous solution, the reaction mixture was diluted with H₂O (10 ml) and extracted with EtOAc (10 ml). The organic phase was washed with H₂O (1×10 ml), brine (2×10 ml), dried over Na₂SO₄, filtered and concentrated. The resulting residue was purified by flash chromatography on silica gel using a gradient of EtOAc in heptane as eluent to yield the title compound (124 mg, 72%) as a pale yellow oil: t_{R}=2.15 min (LC-B); MS (pos.): m/z 289.1 [M+Na]⁺.

### 2b33) Ethyl (6-Formyl-2-methyl-3-propoxy-phenoxy)-acetate

Proceeding in a similar manner to the method described in Example 2b1b, but using 2-hydroxy-3-methyl-4-propoxy-benzaldehyde (Example 2b25) in place of 2,3-dimethyl-4-hydroxy-benzaldehyde, and ethyl bromoacetate in place of 1-bromopropane, gave the title compound: t_{R}=7.00 min (LC-A); MS (pos.): m/z 281.3 [M+H]⁺.

### 2b34) (6-Formyl-2-methyl-3-propoxy-phenoxy)-acetic acid

Proceeding in a similar manner to the method described in Example 2b32, but using ethyl (6-formyl-2-methyl-3-propoxy-phenoxy)-acetate (Example 2b33) in place of 2-(4-acetoxy-butoxy)-3-methyl-4-propoxy-benzaldehyde gave the title compound: t_{R}=6.00 min (LC-A); MS (pos.): m/z 253.3 [M+H]⁺; MS (neg.): m/z 251.3 [M−H]⁻.

### 2b35) 2-(6-Formyl-2-methyl-3-propoxy-phenoxy)-acetamide

Proceeding in a similar manner to the method described in Example 2b1b, but using 2-hydroxy-3-methyl-4-propoxy-benzaldehyde (Example 2b25) in place of 2,3-dimethyl-4-hydroxy-benzaldehyde, and 2-bromoacetamide in place of 1-bromopropane, gave the title compound: t_{R}=1.95 min (LC-B); MS (pos.): m/z 274.3 [M+Na]⁺.

### 2b36) Ethyl 4-(6-Formyl-2-methyl-3-propoxy-phenoxy)-butanoate

Proceeding in a similar manner to the method described in Example 2b1b, but using 2-hydroxy-3-methyl-4-propoxy-benzaldehyde (Example 2b25) in place of 2,3-dimethyl-4-hydroxy-benzaldehyde, and ethyl 4-bromobutanoate in place of 1-bromopropane, gave the title compound: t_{R}=2.56 min (LC-A); MS (pos.): m/z 331.2 [M+Na]⁺.

### 2b37) 4-(6-Formyl-2-methyl-3-propoxy-phenoxy)-butanoic acid

Proceeding in a similar manner to the method described in Example 2b32, but using ethyl 4-(6-formyl-2-methyl-3-propoxy-phenoxy)-butanoate (Example 2b36) in place of 2-(4-acetoxy-butoxy)-3-methyl-4-propoxy-benzaldehyde gave the title compound: t_{R}=2.16 min (LC-B); MS (pos.): m/z 303.2 [M+Na]⁺; MS (neg.): m/z 279.3 [M−H]⁻.

### 2b38) 4-(6-Formyl-2-methyl-3-propoxy-phenoxy)-butanoic acid ethylamide

To a mixture of 4-(6-formyl-2-methyl-3-propoxy-phenoxy)-butanoic acid (Example 2b37, 50 mg, 0.18 mmol), in anhydrous DMF (0.5 ml), were added consecutively DIPEA (122 μl, 0.71 mmol) and HATU (71 mg, 0.19 mmol). After 10 min, ethylamine hydrochloride (18 mg, 0.22 mmol) was added and the reaction mixture was stirred overnight. The reaction mixture was then diluted with H₂O (10 ml) and extracted with EtOAc (10 ml). The organic phase was washed with H₂O (1×10 ml), brine (2×5 ml), dried over Na₂SO₄, filtered and concentrated. The resulting residue was purified by flash chromatography on silica gel using a gradient of EtOAc in heptane as eluent to yield the title compound (124 mg, 72%) as a pale yellow oil: t_{R}=2.18 min (LC-B); MS (pos.): m/z 308.2 [M+H]⁺.

### 2b39) 3-Methyl-2-(4-morpholin-4-yl-4-oxo-butoxy)-4-propoxy-benzaldehyde

Proceeding in a similar manner to the method described in Example 2b38, but using morpholine in place of ethyl amine hydrochloride gave the title compound: t_{R}=2.22 min (LC-B); MS (pos.): m/z 372.2 [M+Na]⁺.

### 2b40) Ethyl 5-(6-Formyl-2-methyl-3-propoxy-phenoxy)-pentanoate

Proceeding in a similar manner to the method described in Example 2b1b, but using 2-hydroxy-3-methyl-4-propoxy-benzaldehyde (Example 2b25) in place of 2,3-dimethyl-4-hydroxy-benzaldehyde, and ethyl 5-bromovalerate in place of 1-bromopropane, gave the title compound: t_{R}=2.62 min (LC-B); MS (pos.): m/z 345.2 [M+Na]⁺.

### 2b41) 5-(6-Formyl-2-methyl-3-propoxy-phenoxy)-pentanoic acid

Proceeding in a similar manner to the method described in Example 2b32, but using ethyl 5-(6-formyl-2-methyl-3-propoxy-phenoxy)-pentanoate (Example 2b40) in place of 2-(4-acetoxy-butoxy)-3-methyl-4-propoxy-benzaldehyde gave the title compound: t_{R}=2.27 min (LC-B); MS (pos.): m/z 317.1 [M+Na]⁺.

### 2b42) 5-(6-Formyl-2-methyl-3-propoxy-phenoxy)-pentanoic acid ethylamide

Proceeding in a similar manner to the method described in Example 2b38, but using 5-(6-formyl-2-methyl-3-propoxy-phenoxy)-pentanoic acid (Example 2b41) in place of 4-(6-formyl-2-methyl-3-propoxy-phenoxy)-butanoic acid gave the title compound: t_{R}=2.24 min (LC-B); MS (pos.): m/z 322.2 [M+H]⁺.

### 2b43) 3-Methyl-2-(5-morpholin-4-yl-5-oxo-pentyloxy)-4-propoxy-benzaldehyde

Proceeding in a similar manner to the method described in Example 2b38, but using 5-(6-formyl-2-methyl-3-propoxy-phenoxy)-pentanoic acid (Example 2b41) in place of 4-(6-formyl-2-methyl-3-propoxy-phenoxy)-butanoic acid and morpholine in place of ethyl amine hydrochloride gave the title compound: t_{R}=2.24 min (LC-B); MS (pos.): m/z 386.2 [M+Na]⁺.

### 2b44) 2-(2-Dimethylamino-ethoxy)-3-methyl-4-propoxy-benzaldehyde hydrochloride

To a solution of 2-hydroxy-3-methyl-4-propoxy-benzaldehyde (Example 2b25, 200 mg, 1.0 mmol), in anhydrous DMF (3 ml) were added consecutively K₂CO₃ (356 mg, 2.58 mmol), potassium iodide (17 mg, 0.1 mmol) and (2-chloro-ethyl)-dimethyl-amine hydrochloride (163 mg, 1.10 mmol). The mixture was heated at 60° C. overnight. After cooling to room temperature, the mixture was diluted with H₂O (10 ml) and extracted with EtOAc (2×100 ml). The combined organic phases were washed with H₂O (2×5 ml), brine (2×5 ml), dried over Na₂SO₄, filtered and concentrated. The resulting residue was purified by thick layer chromatography on silica gel using a mixture of CH₂Cl₂/MeOH/Et₃N 100/10/1 as eluent to yield 2-(2-dimethylamino-ethoxy)-3-methyl-4-propoxy-benzaldehyde (139 mg, 50%) as a pale yellow oil.

To a solution of 2-(2-dimethylamino-ethoxy)-3-methyl-4-propoxy-benzaldehyde (139 mg, 0.52 mmol) in 1,4-dioxane was added a 4N HCl solution in 1,4-dioxane (0.20 ml, 0.8 mmol). The volatiles were then evaporated in vacuo to yield the title compound (159 mg, 100%) as a brown solid: t_{R}=0.99 min, 4.44 min (LC-A); MS (pos.): m/z 266.2 [M+H]⁺.

### 2b45) 3-Methyl-2-(2-morpholin-4-yl-ethoxy)-4-propoxy-benzaldehyde hydrochloride

Proceeding in a similar manner to the method described in Example 2b44, but using 4-(2-chloro-ethyl)-morpholine hydrochloride in place of (2-chloro-ethyl)-dimethyl-amine hydrochloride gave the title compound: t_{R}=0.37 min, 1.52 min (LC-B); MS (pos.): m/z 308.2 [M+H]⁺; MS (neg.): m/z 306.3 [M−H]⁻.

### 2b46) 3-Methyl-2-(2-piperidin-1-yl-ethoxy)-4-propoxy-benzaldehyde hydrochloride

Proceeding in a similar manner to the method described in Example 2b44, but using 1-(2-chloro-ethyl)-piperidine hydrochloride in place of (2-chloro-ethyl)-dimethyl-amine hydrochloride gave the title compound: Method B t_{R}=0.36 min, 1.56 min (LC-B); MS (pos.): m/z 306.3 [M+H]⁺.

### 2b47) 2-(3-Dimethylamino-propoxy)-3-methyl-4-propoxy-benzaldehyde hydrochloride

Proceeding in a similar manner to the method described in Example 2b44, but using (3-chloro-propyl)-dimethyl-amine hydrochloride in place of (2-chloro-ethyl)-dimethyl-amine hydrochloride gave the title compound: Method B t_{R}=0.37 min, 1.58 min (LC-B); MS (pos.): m/z 280.3 [M+H]⁺.

### 2b48) 2-(2,2-Dimethyl-[1,3]dioxolan-4-ylmethoxy)-3-methyl-4-propoxy-benzaldehyde

To a solution of 2-hydroxy-3-methyl-4-propoxy-benzaldehyde (Example 2b25, 400 mg, 2.1 mmol), in anhydrous DMF (4 ml) were added consecutively K₂CO₃ (427 mg, 3.09 mmol), potassium iodide (35 mg, 0.21 mmol) and 2,2-dimethyl-1,3-dioxolan-4-ylmethyl p-toluenesulfonate (650 mg, 2.27 mmol). The reaction mixture was heated at 90° C. overnight. After cooling to room temperature, the mixture was diluted with H₂O (20 ml) and extracted with EtOAc (2×30 ml). The combined organic phases were washed with H₂O (2×10 ml), brine (2×10 ml), dried over Na₂SO₄, filtered and concentrated. The resulting residue was purified by flash chromatography on silica gel using a gradient of EtOAc in heptane as eluent to yield the title compound (340 mg, 54%) as a pale yellow oil: t_{R}=2.51 min (LC-B); MS (pos.): m/z 331.2 [M+Na]⁺.

### 2b49) 2-((4R)-2,2-Dimethyl-[1,3]dioxolan-4-ylmethoxy)-3-methyl-4-propoxy-benzaldehyde

Proceeding in a similar manner to the method described in Example 2b48, but using (4S)-2,2-dimethyl-1,3-dioxolan-4-ylmethyl p-toluenesulfonate in place of 2,2-dimethyl-1,3-dioxolan-4-ylmethyl p-toluenesulfonate gave the title compound: t_{R}=2.45 (LC-B); MS (pos.): m/z 331.2 [M+Na]⁺.

### 2b50) 2-((4S)-2,2-Dimethyl-[1,3]dioxolan-4-ylmethoxy)-3-methyl-4-propoxy-benzaldehyde

Proceeding in a similar manner to the method described in Example 2b48, but using (4R)-2,2-dimethyl-1,3-dioxolan-4-ylmethyl p-toluenesulfonate in place of 2,2-dimethyl-1,3-dioxolan-4-ylmethyl p-toluenesulfonate gave the title compound: t_{R}=2.46 (LC-B); MS (pos.): m/z 331.2 [M+Na]⁺.

### 2b51) Preparation of ethyl (3R)-4-(6-formyl-2-methyl-3-propoxy-phenoxy)-3-hydroxy-butanoate

### 2b51a) Ethyl (3R)-3-(tert-butyl-dimethylsilanyloxy)-4-(6-formyl-2-methyl-3-propoxy-phenoxy)-butanoate

Proceeding in a similar manner to the method described in Example 2b1b, but using 2-hydroxy-3-methyl-4-propoxy-benzaldehyde (Example 2b25) in place of 2,3-dimethyl-4-hydroxy-benzaldehyde, and ethyl (3R)-3-(tert-butyl-dimethyl-silanyloxy)-4-iodo-butanoate, prepared according to Hareau G. P-J. et al., J. Am. Chem. Soc. 1999, 3640-3650, in place of 1-bromopropane, gave the title compound: t_{R}=3.15 min (LC-B); MS (pos.): m/z 461.4 [M+Na]⁺.

### 2b51b) Ethyl (3R)-4-(6-formyl-2-methyl-3-propoxy-phenoxy)-3-hydroxy-butanoate

To a solution of ethyl (3R)-3-(tert-butyldimethylsiloxy)-4-(6-formyl-2-methyl-3-propoxy-phenoxy)-butanoate (Example 2b51a, 200 mg, 0.46 mmol), in THF (0.8 ml) at room temperature was added a 1M solution of TBAF in THF (684 μl, 0.68 mmol). The reaction mixture was stirred overnight and then diluted with H₂O (15 ml). After extraction with EtOAc (25 ml), the organic phase was washed with H₂O (2×5 ml), brine (2×5 ml), dried over Na₂SO₄, filtered and concentrated. The resulting residue was purified by flash chromatography on silica gel using a gradient of EtOAc in heptane as eluent to yield the title compound (78 mg, 53%) as a pale yellow oil: t_{R}=2.24 min (LC-B); MS (pos.): m/z 347.3 [M+Na]⁺.

### 2b52) (3R)-4-(6-Formyl-2-methyl-3-propoxy-phenoxy)-3-hydroxy-butanoic acid

Proceeding in a similar manner to the method described in Example 2b32, but using ethyl (3R)-4-(6-formyl-2-methyl-3-propoxy-phenoxy)-3-hydroxy-butanoate (Example 2b51b) in place of 2-(4-acetoxy-butoxy)-3-methyl-4-propoxy-benzaldehyde gave the title compound: t_{R}=1.96 min (LC-B); MS (pos.): m/z 319.2 [M+Na]⁺; MS (neg.): m/z 295.2 [M−H]⁻.

### 2b53) Preparation of ethyl (3S)-4-(6-formyl-2-methyl-3-propoxy-phenoxy)-3-hydroxy-butanoate

### 2b53a) Ethyl (3S)-3-(tert-butyl-dimethylsilanyloxy)-4-(6-formyl-2-methyl-3-propoxy-phenoxy)-butanoate

Proceeding in a similar manner to the method described in Example 2b1b, but using 2-hydroxy-3-methyl-4-propoxy-benzaldehyde) (Example 2b25) in place of 2,3-dimethyl-4-hydroxy-benzaldehyde, and ethyl (3S)-3-(tert-butyl-dimethylsilanyloxy)-4-bromobutanoate in place of 1-bromopropane, gave the title compound: t_{R}=3.19 min (LC-B); MS (pos.): m/z 461.4 [M+Na]⁺.

### 2b53b) Ethyl (3S)-4-(6-formyl-2-methyl-3-propoxy-phenoxy)-3-hydroxy-butanoate

Proceeding in a similar manner to the method described in Example 2b51b, but using ethyl (3S)-3-(tert-butyl-dimethylsilanyloxy)-4-(6-formyl-2-methyl-3-propoxy-phenoxy)-butanoate (Example 2b53a) in place of ethyl (3R)-3-(tert-butyl-dimethylsilanyloxy)-4-(6-formyl-2-methyl-3-propoxy-phenoxy)-butanoate gave the title compound: t_{R}=2.24 min (LC-B); MS (pos.): m/z 347.3 [M+Na]⁺.

### 2b54) (3S)-4-(6-Formyl-2-methyl-3-propoxy-phenoxy)-3-hydroxy-butanoic acid

Proceeding in a similar manner to the method described in Example 2b32, but using ethyl (3S)-4-(6-formyl-2-methyl-3-propoxy-phenoxy)-3-hydroxy-butanoate (Example 2b53b) in place of 2-(4-acetoxy-butoxy)-3-methyl-4-propoxy-benzaldehyde gave the title compound: t_{R}=2.33 min (LC-B); MS (pos.): m/z 319.2 [M+Na]⁺; MS (neg.): m/z 295.2 [M−H]⁻.

### 2b55) Methyl (4R,5S)-5-(6-formyl-2-methyl-3-propoxy-phenoxymethyl)-2,2-dimethyl-[1,3]dioxolane-4-carboxylate

Proceeding in a similar manner to the method described in Example 2b1b, but using 2-hydroxy-3-methyl-4-propoxy-benzaldehyde (Example 2b25) in place of 2,3-dimethyl-4-hydroxy-benzaldehyde, and methyl (4R,5S)-5-tosyloxymethyl-2,2-dimethyl-1,3-dioxolane-4-carboxylate, prepared from dimethyl 2,3-O-isopropylidene-L-tartrate in two steps according to Batsanov A. S. et al., J. Chem. Soc., Perkin Trans. 1, 1995, 1281-1294 and Ortuno R. M. et al., Tetrahedron 1997, 2191-2198, in place of 1-bromopropane, gave the title compound: t_{R}=2.48 min (LC-B); MS (pos.): m/z 389.2 [M+Na]⁺.

### 2b56) (4R,5S)-5-(6-Formyl-2-methyl-3-propoxy-phenoxymethyl)-2,2-dimethyl-[1,3]dioxolane-4-carboxylic acid

Proceeding in a similar manner to the method described in Example 2b32, but using methyl (4R,5S)-5-(6-formyl-2-methyl-3-propoxy-phenoxymethyl)-2,2-dimethyl-[1,3]dioxolane-4-carboxylate (Example 2b55) in place of 2-(4-acetoxy-butoxy)-3-methyl-4-propoxy-benzaldehyde gave the title compound: t_{R}=2.48 min (LC-B); MS (neg.): m/z 351.3 [M−H]⁻.

### Example 3

### R₁ is 4-Ethoxycarbonylphenyl

3a) The following products 3a1-3a7 were prepared by proceeding in a similar manner to the method described in Example 1, but using ethyl 4-(3,5-dioxo-pyrazolidin-1-yl)-benzoate (Example 3b2) in place of 1-phenyl-pyrazolidine-3,5-dione and the respective aldehydes:

3a1) Ethyl 4-(4-benzylidene-3,5-dioxo-pyrazolidin-1-yl)-benzoate, from benzaldehyde (Fluka): t_{R}=6.73 nm (LC-A); MS (pos.): m/z 337.3 [M+H]⁺; MS (neg.): m/z 335.5 [M−H]⁻.

3a2) Ethyl 4-[4-(2-hydroxy-3-methoxy-benzylidene)-3,5-dioxo-pyrazolidin-1-yl]-benzoate, from 2-hydroxy-3-methoxybenzaldehyde (Acros): t_{R}=6.34 min (LC-A); MS (pos.): m/z 383.3 [M+H]⁺; MS (neg.): m/z 381.5 [M−H]⁻.

3a3) Ethyl 4-[4-(2-methoxy-benzylidene)-3,5-dioxo-pyrazolidin-1-yl]-benzoate, from 2-methoxybenzaldehyde (Acros): t_{R}=6.77 min (LC-A); MS (pos.): m/z 367.3 [M+H]⁺; MS (neg.): m/z 365.5 [M−H]⁻.

3a4) Ethyl 4-[4-(3-methoxy-benzylidene)-3,5-dioxo-pyrazolidin-1-yl]-benzoate, from 3-methoxybenzaldehyde (Acros): MS (pos.): t_{R}=6.84 min (LC-A); m/z 367.3 [M+H]⁺; MS (neg.): m/z 365.5 [M−H]⁻.

3a5) Ethyl 4-(3,5-dioxo-4-pyridin-3-ylmethylene-pyrazolidin-1-yl)-benzoate, from 3-pyridinecarboxaldehyde (Acros): t_{R}=5.64 min (LC-A); MS (pos.): m/z 338.5 [M+H]⁺; MS (neg.): m/z 336.5 [M−H]⁻.

3a6) Ethyl 4-(3,5-dioxo-4-thiophen-3-ylmethylene-pyrazolidin-1-yl)-benzoate, from 3-thiophencarboxaldehyde (Aldrich) t_{R}=6.59 min (LC-A); MS (pos.): m/z 343.3 [M+H]⁺; MS (neg.): m/z 341.5 [M−H]⁻.

3a7) Ethyl 4-[4-(2,3-dimethyl-4-propoxy-benzylidene)-3,5-dioxo-pyrazolidin-1-yl]-benzoate, from 2,3-dimethyl-4-propoxybenzaldehyde (Example 2b1b): t_{R}=7.89 min (LC-A); MS (pos.): m/z 423.4 [M+H]⁺; MS (neg.): m/z 421.6 [M−H]⁻.

### 3b) Preparation of ethyl 4-(3,5-dioxo-pyrazolidin-1-yl)-benzoate

### 3b1) Ethyl 4-[N′-(2-ethoxycarbonyl-acetyl)-hydrazino]-benzoate

(4-Hydrazino)ethylbenzoate hydrochoride (12 g, 551.4 mmol, prepared according to Coquet G. et al., Tetrahedron 2000, 56, 2975-2984) was stirred for 15 min at room temperature in a mixture of 10% aqueous Na₂CO₃ solution (100 ml) and CH₂Cl₂ (200 ml). The separated aqueous solution was extracted with CH₂Cl₂ (3×200 ml). The combined organic phases were dried over Na₂SO₄, filtered and concentrated. The residue was dissolved in anhydrous THF (100 ml) and Et₃N (8.11 ml, 58.3 mmol) was added. The reaction mixture was cooled to −10° C. and a solution of ethyl malonyl chloride (7.12 ml, 56.6 mmol) in anhydrous THF (50 ml) was added dropwise. The reaction mixture was allowed to warm to room temperature and stirred overnight. The reaction mixture was diluted with H₂O (100 ml) and extracted with EtOAc (3×100 ml). The combined organic phases were washed with H₂O (100 ml), brine (100 ml), dried over Na₂SO₄, filtered and concentrated to yield crude title compound as a brown residue (16.3 g): t_{R}=5.34 min (LC-A); MS (pos.): m/z 295.1 [M+H]⁺; MS (neg.): m/z 293.3 [M−H]⁻.

### 3b2) Ethyl 4-(3,5-dioxo-pyrazolidin-1-yl)-benzoate

Crude ethyl 4-[N′-(2-ethoxycarbonyl-acetyl)-hydrazino]-benzoate (Example 3b1, 16.3 g) was dissolved in EtOH (100 ml) and an ethanolic 1N NaOH solution (110 ml, 110 mmol) was added. The reaction mixture was stirred 30 min at room temperature. The reaction mixture was then acidified by addition of aqueous 1N HCl. The precipitate formed was collected by filtration, washed with H₂O (3×20 ml), dried on the sintered glass and in vacuo to yield the title compound as an off-white solid (8.27 g, 60%): t_{R}=4.50 min (LC-A); MS (pos.): m/z 249.2 [M+H]⁺; MS (neg.): m/z 247.3 [M−H]⁻.

### Example 4

### R₁ is 2-Pyridyl

4a) The following products 4a1 and 4a2 were prepared by proceeding in a similar manner to the method described in Example 1, but using 1-pyridin-2-yl-pyrazolidine-3,5-dione (Example 4b2) in place of 1-phenyl-pyrazolidine-3,5-dione, and the respective aldehydes:

4a1) 4-(3-Methyl-4-propoxy-benzylidene)-1-pyridin-2-yl-pyrazolidine-3,5-dione, from 3-methyl-4-propoxybenzaldehyde (Example 2b3): t_{R}=7.30 min (LC-A); MS (pos.): m/z 338.3 [M+H]⁺; MS (neg.): m/z 336.3 [M−H]⁻.

4a2) 4-(2,3-Dimethyl-4-propoxy-benzylidene)-1-pyridin-2-yl-pyrazolidine-3,5-dione, from 2,3-dimethyl-4-propoxybenzaldehyde (Example 2b1b): t_{R}=7.44 min (LC-A); MS (pos.): m/z 352.2 [M+H]⁺; MS (neg.): m/z 350.3 [M−H]⁻.

### 4b) Preparation of 1-pyridin-2-yl-pyrazolidine-3,5-dione

### 4b1) Ethyl (N′-pyridin-2-yl-hydrazinocarbonyl)-acetate

To a solution of 2-hydrazinopyridine (2.0 g, 18.3 mmol) and Et₃N (2.68 ml, 19.2 mmol) in anhydrous THF (30 ml) cooled at −10° C. was added dropwise ethyl malonyl chloride (2.35 ml, 18.7 mmol). The reaction mixture was allowed to warm to room temperature and stirred overnight. The reaction mixture was diluted with H₂O (20 ml) and extracted with EtOAc (2×20 ml). The combined organic phases were washed with brine (20 ml), dried over Na₂SO₄, filtered and concentrated. The resulting residue was purified by flash chromatography on silica gel using a gradient of EtOAc in heptane as eluent to yield the title compound (2.11 g, 52%) as a brown solid: t_{R}=0.98 min (LC-A); MS (pos.): m/z 224.5 [M+H]⁺; MS (neg.): m/z 222.5 [M−H]⁻.

### 4b2) 1-Pyridin-2-yl-pyrazolidine-3,5-dione

Ethyl (N′-pyridin-2-yl-hydrazinocarbonyl)-acetate (Example 4b1, 1.0 g, 4.48 mmol) was dissolved in a 1N ethanolic NaOH solution (9 ml, 9 mmol). The reaction mixture was stirred 30 min at room temperature, acidified by addition of AcOH, then diluted with H₂O (20 ml) and extracted with CH₂Cl₂ (2×30 ml). The combined organic phases were washed with H₂O (30 ml), dried over Na₂SO₄, filtered and concentrated to yield the title compound (500 mg, 63%) as a yellow solid: t_{R}=2.26 min (LC-A); MS (pos.): m/z 178.2 [M+H]⁺; MS (neg.): m/z 176.3 [M−H]⁻.

### Example 5

### R₁ is 4-Bromophenyl

### 5a) 1-(4-Bromo-phenyl)-4-(2,3-dimethyl-4-propoxy-benzylidene)-pyrazolidine-3,5-dione

Proceeding in a similar manner to the method described in Example 1, but using 1-(4-bromo-phenyl)-pyrazolidine-3,5-dione (Example 5b2) in place of 1-phenyl-pyrazolidine-3,5-dione, gave the title compound: t_{R}=7.95 min (LC-A); MS (pos.): m/z 429.3, 431.2 [M+H]⁺; MS (neg.): m/z 427.5, 429.4 [M−H]⁻.

### 5b) Preparation of 1-(4-bromo-phenyl)-pyrazolidine-3,5-dione

### 5b1) Ethyl [N′-(4-bromo-phenyl)-hydrazinocarbonyl]-acetate

Proceeding in a similar manner to the method described in Example 4b1, but using 4-bromophenylhydrazine hydrochloride in place of 2-hydrazinopyridine, gave the title compound: t_{R}=5.53 min (LC-A); MS (pos.): m/z 301.0, 303.1 [M+H]⁺; MS (neg.): m/z 299.1, 301.2 [M−H]⁻.

### 5b2) 1-(4-Bromo-phenyl)-pyrazolidine-3,5-dione

Proceeding in a similar manner to the method described in Example 3b2, but using ethyl [N′-(4-bromo-phenyl)-hydrazinocarbonyl]-acetate (Example 5b1) in place of ethyl 4-[N′-(2-ethoxycarbonyl-acetyl)-hydrazino]-benzoate, gave the title: t_{R}=4.96 min (LC-A); MS (pos.): m/z 255.3, 257.3 [M+H]⁺; MS (neg.): m/z 253.4, 255.4 [M−H]⁻.

### Example 6

### R₁ is 4-Methoxyphenyl

### 6a) 1-(4-Methoxy-phenyl)-4-(2,3-dimethyl-4-propoxy-benzylidene)-pyrazolidine-3,5-dione

Proceeding in a similar manner to the method described in Example 1, but using 1-(4-methoxy-phenyl)-pyrazolidine-3,5-dione (Example 6b2) in place of 1-phenyl-pyrazolidine-3,5-dione gave the title compound: t_{R}=7.22 min (LC-A); MS (pos.): m/z 381.4 [M+H]⁺; MS (neg.): m/z 379.6 [M−H]⁻.

### 6b) Preparation of 1-(4-methoxy-phenyl)-pyrazolidine-3,5-dione

### 6b1) Ethyl [N′-(4-methoxy-phenyl)-hydrazinocarbonyl]-acetate

Proceeding in a similar manner to the method described in Example 4b1, but using 4-methoxyphenylhydrazine hydrochloride in place of 2-hydrazinopyridine, gave the title compound: MS (pos.): m/z 253.1 [M+H]⁺.

### 6b2) 1-(4-Methoxy-phenyl)-pyrazolidine-3,5-dione

Proceeding in a similar manner to the method described in Example 4b2, but using ethyl [N′-(4-methoxy-phenyl)-hydrazinocarbonyl]-acetate (Example 6b1) in place of ethyl (N′-pyridin-2-yl-hydrazinocarbonyl)-acetate, gave the title compound: t_{R}=3.85 min (LC-A); MS (pos.): m/z 206.9 [M+H]⁺.

### Example 7

### R₁ is 4-Cyanophenyl

### 7a) 4-[4-(2,3-Dimethyl-4-propoxy-benzylidene)-3,5-dioxo-pyrazolidin-1-yl]-benzonitrile

Proceeding in a similar manner to the method described in Example 1, but using 4-(3,5-dioxo-pyrazolidin-1-yl)-benzonitrile (Example 7b2) in place of 1-phenyl-pyrazolidine-3,5-dione, gave the title compound: t_{R}=7.58 min (LC-A); MS (pos.): m/z 376.4 [M+H]⁺; MS (neg.): m/z 374.6 [M−H]⁻.

### 7b) Preparation of 4-(3,5-Dioxo-pyrazolidin-1-yl)-benzonitrile

### 7b1) Ethyl [N′-(4-cyano-phenyl)-hydrazinocarbonyl]-acetate

Proceeding in a similar manner to the method described in Example 4b1, but using 4-cyanophenylhydrazine hydrochloride in place of 2-hydrazinopyridine, gave the title compound: t_{R}=4.67 min (LC-A); MS (pos.): m/z 247.8 [M+H]⁺; MS (neg.): m/z 245.9 [M−H]⁻.

### 7b2) 4-(3,5-Dioxo-pyrazolidin-1-yl)-benzonitrile

Proceeding in a similar manner to the method described in Example 4b2, but using ethyl [N′-(4-cyano-phenyl)-hydrazinocarbonyl]-acetate (Example 7b1) in place of ethyl (N′-pyridin-2-yl-hydrazinocarbonyl)-acetate, gave the title compound: t_{R}=4.17 min (LC-A); MS (pos.): m/z 201.9 [M+H]⁺; MS (neg.): m/z 199.9 [M−H]⁻.

### Example 8

### R₁ is 4-Fluorophenyl

### 8a) 4-(2,3-Dimethyl-4-propoxy-benzylidene)-1-(4-fluoro-phenyl)-pyrazolidine-3,5-dione

Proceeding in a similar manner to the method described in Example 1, but using 1-(4-fluoro-phenyl)-pyrazolidine-3,5-dione (Example 8b2) in place of 1-phenyl-pyrazolidine-3,5-dione, gave the title compound: t_{R}=7.51 min (LC-A); MS (pos.): m/z 369.4 [M+H]⁺; MS (neg.): m/z 367.6 [M−H]⁻.

### 8b) Preparation of 1-(4-Fluoro-phenyl)-pyrazolidine-3,5-dione

### 8b1) Ethyl [N′-(4-fluoro-phenyl)-hydrazinocarbonyl]-acetate

Proceeding in a similar manner to the method described in Example 4b1, but using 4-fluorophenylhydrazine hydrochloride in place of 2-hydrazinopyridine, gave the title compound: t_{R}=4.55 min (LC-A); MS (neg.): m/z 238.9 [M−H]⁻.

### 8b2) 1-(4-Fluoro-phenyl)-pyrazolidine-3,5-dione

Proceeding in a similar manner to the method described in Example 4b2, but using ethyl [N′-(4-fluoro-phenyl)-hydrazinocarbonyl]-acetate (Example 8b1) in place of ethyl (N′-pyridin-2-yl-hydrazinocarbonyl)-acetate, gave the title compound: t_{R}=3.93 min (LC-A); MS (pos.): m/z 195.0 [M+H]⁺; MS (neg.): m/z 193.0 [M−H]⁻.

### Example 9

### R₁ is 4-Methylphenyl

### 9a) 4-(2,3-Dimethyl-4-propoxy-benzylidene)-1-(4-methyl-phenyl)-pyrazolidine-3,5-dione

Proceeding in a similar manner to the method described in Example 1, but using 1-(4-methyl-phenyl)-pyrazolidine-3,5-dione (Example 9b2) in place of 1-phenyl-pyrazolidine-3,5-dione, gave the title compound: t_{R}=7.65 min (LC-A); MS (pos.): m/z 365.4 [M+H]⁺; MS (neg.): m/z 363.6 [M−H]⁻.

### 9b) Preparation of 1-(4-methyl-phenyl)-pyrazolidine-3,5-dione

### 9b1) Ethyl [N′-(4-methyl-phenyl)-hydrazinocarbonyl]-acetate

Proceeding in a similar manner to the method described in Example 4b1, but using 4-methylphenylhydrazine hydrochloride in place of 2-hydrazinopyridine, gave the title compound: t_{R}=5.18 min (LC-A); MS (neg.): m/z 235.0 [M−H]⁻.

### 9b2) 1-(4-Methyl-phenyl)-pyrazolidine-3,5-dione

Proceeding in a similar manner to the method described in Example 4b2, but using ethyl [N′-(4-methyl-phenyl)-hydrazinocarbonyl]-acetate (Example 9b1) in place of ethyl (N′-pyridin-2-yl-hydrazinocarbonyl)-acetate, gave the title compound: t_{R}=4.34 min (LC-A); MS (pos.): m/z 190.8 [M+H]⁺; MS (neg.): m/z 188.9 [M−H]⁻.

### Example 10

### R₁ is 2-Chlorophenyl

### 10a) 1-(2-Chloro-phenyl)-4-(2,3-dimethyl-4-propoxy-benzylidene)-pyrazolidine-3,5-dione

A mixture of 1-(2-chloro-phenyl)-pyrazolidine-3,5-dione (Example 10b, 64 mg, 0.3 mmol) and 2,3-dimethyl-4-propoxybenzaldehyde (Example 2b1, 87 mg, 0.45 mmol) in absolute ethanol (4 ml) was heated at reflex for 16 h under inort atmosphere. After cooling to room temperature, the solvent was evaporated in vacuo. The resulting residue was purified by flash chromatography on silica gel using a gradient of EtOAc in heptane as eluent to yield the title compound (62 mg, 54%) as an orange-red solid: t_{R}=7.16 min (LC-A); MS (pos.): m/z 385.4 [M+H]⁺; MS (neg.): m/z 383.5 [M−H]⁻.

### 10b) 1-(2-Chloro-phenyl)-pyrazolidine-3,5-dione

To a solution of sodium ethoxide (42 mmol) in absolute ethanol (22 ml) were added diethylmalonate (2.12 ml, 14.0 mmol) and 2-chlorophenylhydrazine hydrochloride (2.5 g, 14.0 mmol). The volatiles were immediately distilled at atmospheric pressure and the resulting residue was further heated at 140° C. to dryness. After cooling to room temperature, the residue was dissolved in water (50 ml). Neutral was removed from the aqueous solution by extraction with diethyl ether (2×100 ml). The aqueous phase was then acidified to pH 1 by addition of 1N aqueous HCl and extracted with EtOAc (3×100 ml). The combined organic phases were washed with H₂O (50 ml), brine (50 ml), dried over MgSO₄, filtered and concentrated. The resulting residue was purified by flash chromatography on silica gel using a gradient of MeOH in CH₂Cl₂ as eluent to yield the title compound (500 mg, 17%) as a pale brown solid: t_{R}=3.48 min (LC-A); MS (pos.): m/z 211.1 [M+H]⁺; MS (neg.): m/z 208.8 [M−H]⁻.

### Example 11

### R₁ is 2-Methylphenyl

### 11a) 4-(2,3-Dimethyl-4-propoxy-benzylidene)-1-(2-methyl-phenyl)-pyrazolidine-3,5-dione

Proceeding in a similar manner to the method described in Example 10a, but using 1-(2-methyl-phenyl)-pyrazolidine-3,5-dione (Example 11b) in place of 1-(2-chloro-phenyl)-pyrazolidine-3,5-dione, gave the title compound: t_{R}=7.16 min (LC-A); MS (pos.): m/z 365.5 [M+H]⁺; MS (neg.): m/z 363.6 [M−H]⁻.

### 11b) 1-(2-Methyl-phenyl)-pyrazolidine-3,5-dione

Proceeding in a similar manner to the method described in Example 10b, but using 2-methylphenylhydrazine hydrochloride in place of 2-chlorophenylhydrazine hydrochloride, gave the title compound: t_{R}=3.45 min (LC-A); MS (pos.): m/z 191.3 [M+H]⁺.

### Example 12

### R₁ is H

The following products 12a1-12a9 were prepared by proceeding in a similar manner to the method described in Example 1, but using pyrazolidine-3,5-dione (prepared according to Fritsch G. et al., Arch. Pharm., Weinheim Ger. 1986, 70-78) in place of 1-phenyl-pyrazolidine-3,5-dione and the respective aldehydes:

12a1) 4-(2,3-Dimethyl-4-propoxy-benzylidene)-pyrazolidine-3,5-dione, from 2,3-dimethyl-4-propoxybenzaldehyde (Example 2b1b): t_{R}=5.91 min (LC-A); MS (pos.): m/z 275.4 [M+H]⁺; MS (neg.): m/z 273.5 [M−H]⁻.

12a2) 4-(4-Cyclopentyloxy-2,3-dimethyl-benzylidene)-pyrazolidine-3,5-dione, from 4-cyclopentyloxy-2,3-dimethyl-benzaldehyde (Example 2b14): t_{R}=6.29 min (LC-A); MS (pos.): m/z 301.5 [M+H]⁺; MS (neg.): m/z 299.6 [M−H]⁻.

12a3) 4-(4-Propoxy-5,6,7,8-tetrahydro-naphthalen-1-ylmethylene)-pyrazolidine-3,5-dione, from 4-propoxy-5,6,7,8-tetrahydro-naphthalene-1-carbaldehyde (Example 2b15c): t_{R}=6.36 min (LC-A); MS (pos.): m/z 301.2 [M+H]⁺; MS (neg.): m/z 299.3 [M−H]⁻.

12a4) 4-(2,3-Dimethyl-4-pent-1-ynyl-benzylidene)-pyrazolidine-3,5-dione, from 2,3-dimethyl-4-pent-1-ynyl-benzaldehyde (Example 2b16b): t_{R}=6.32 min (LC-A); MS (pos.): m/z 283.2 [M+H]⁺; MS (neg.): m/z 281.3 [M−H]⁻.

12a5) 4-(2,3-Dimethyl-4-pentyl-benzylidene)-pyrazolidine-3,5-dione, from 2,3-dimethyl-4-pentyl-benzaldehyde (Example 2b16c): t_{R}=6.64 min (LC-A); MS (pos.): m/z 287.2 [M+H]⁺; MS (neg.): m/z 285.3 [M−H]⁻.

12a6) 4-(4-Propoxy-naphthalen-1-ylmethylene)-pyrazolidine-3,5-dione, from 4-propoxy-1-naphtaldehyde (Example 2b17): t_{R}=6.13 min (LC-A); MS (pos.): m/z 297.2 [M+H]⁺; MS (neg.): m/z 295.3 [M−H]⁻.

12a7) 4-(7-Propoxy-indan-4-ylmethylene)-pyrazolidine-3,5-dione, from 7-propoxy-indan-4-carbaldehyde (Example 2b18e): t_{R}=6.11 min (LC-A); MS (pos.): m/z 287.3 [M+H]⁺; MS (neg.): m/z 285.3 [M−H]

12a8) 4-(2-Methoxy-3-methyl-4-propoxy-benzylidene)-pyrazolidine-3,5-dione, from 2-methoxy-3-methyl-4-propoxy-benzaldehyde (Example 2b26): t_{R}=6.02 min (LC-A); MS (pos.): m/z 291.3 [M+H]⁺; MS (neg.): m/z 289.4 [M−H]⁻.

12a9) 4-(3-Methyl-2,4-dipropoxy-benzylidene)-pyrazolidine-3,5-dione, from 3-methyl-2,4-dipropoxy-benzaldehyde (Example 2b27): t_{R}=6.59 min (LC-A); MS (pos.): m/z 319.3 [M+H]⁺; MS (neg.): m/z 317.4 [M−H]⁻.

### Example 13

### R₁ is Methyl

### 13a) 4-(2,3-Dimethyl-4-propoxy-benzylidene)-1-methyl-pyrazolidine-3,5-dione

Proceeding in a similar manner to the method described in Example 1, but using 1-methyl-pyrazolidine-3,5-dione (Example 13b) in place of 1-phenyl-pyrazolidine-3,5-dione, gave the title compound: t_{R}=6.15 min (LC-A); MS (pos.): m/z 289.5 [M+H]⁺; MS (neg.): m/z 287.5 [M−H]⁻.

### 13b) 1-Methyl-pyrazolidine-3,5-dione

Proceeding in a similar manner to the method described in Example 10b, but using methylhydrazine in place of 2-chlorophenylhydrazine hydrochloride, gave the title compound: t_{R}=0.93 min (LC-A); MS (pos.): m/z 115.2 [M+H]⁺; MS (neg.): m/z 113.3 [M−H]⁻.

### Example 14

### R₁ is 4-Pyridyl

### 14a) 4-(2,3-Dimethyl-4-propoxy-benzylidene)-1-pyridin-4-yl-pyrazolidine-3,5-dione

Proceeding in a similar manner to the method described in Example 10a, but using 1-pyridin-4-yl-pyrazolidine-3,5-dione (Example 14b) in place of 1-(2-chloro-phenyl)-pyrazolidine-3,5-dione, gave the title compound: t_{R}=5.56 min (LC-A); MS (pos.): m/z 352.5 [M+H]⁺; MS (neg.): m/z 350.6 [M−H]⁻.

### 14b) 1-Pyridin-4-yl-pyrazolidine-3,5-dione

Proceeding in a similar manner to the method described in Example 10b, but using 4-pyridylhydrazine hydrochloride (prepared according to Mann F. G. et al., J. Chem. Soc. 1959, 3830-3834) in place of 2-chlorophenylhydrazine hydrochloride, gave the title compound: t_{R}=0.93 min (LC-A); MS (neg.): m/z 176.4 [M−H]⁻.

### 15) 1-Acetyl-4-[1-(2,3-dimethyl-4-propoxy-phenyl)-methylidene]-pyrazolidine-3,5-dione

A mixture of pyrazolidine-3,5-dione (50 mg, 0.5 mmol, prepared according to Fritsch G. et al., Arch. Pharm., Weinheim Ger. 1986, 70-78) and 2,3-dimethyl-4-propoxybenzaldehyde (Example 2b1b, 96 mg, 0.5 mmol) in acetic acid (1.5 ml) and acetic anhydride (200 μl) was stirred at room temperature for 3 days. The formed precipitate was collected by filtration. The solid was washed with H₂O (2×4 ml), with ethanol (2×4 ml) and dried in vacuo to give the title compound (26 mg, 16%) as a yellow solid: t_{R}=6.87 min (LC-A); MS (pos.): m/z 317.3 [M+H]⁺; MS (neg.): m/z 315.4 [M−H]⁻.

NMR data of selected compounds are given below.

| Example | Chemical shifts (δ) in parts per million (ppm) | Solvent |
|---|---|---|
| 1 | 1.00(t, 3H), 1.77(m, 2H), 2.15(s, 3H), 2.37(s, | DMSO-d₆ |
| | 1.5H), 2.38(s, 1.5H), 3.30(br s, 1H | |
| | exchangeable), 4.04(t, 2H), 6.92(m, 1H), | |
| | 7.15(t, 1H), 7.38(m, 2H), 7.69(d, 2H), 8.08(s, | |
| | 0.5H), 8.15(s, 0.5H), 8.73(br s, 0.5H), 8.85(d, | |
| | 0.5H) | |
| 2a25 | 1.00(t, 3H), 1.73(m, 6H), 2.58(m, 2H), 2.88(m, | DMSO-d₆ |
| | 2H), 4.04(t, 2H), 6.85(m, 1H), 7.15(t, 1H), | |
| | 7.40(m, 2H), 7.70(d, 2H), 8.08(s, 0.5H), | |
| | 8.10(s, 0.5H), 8.85(br s, 0.5H), 8.96(d, 0.5H), | |
| | 11.10(br s, 1H) | |
| 2a34 | 1.00(t, 3H), 1.04(t, 3H), 1.80(m, 4H), 3.30(br | DMSO-d₆ |
| | s, 1H exchangeable), 4.08(m, 4H), 6.60(s, 1H), | |
| | 6.70(m, 1H), 7.15(t, 1H), 7.40(t, 2H), 7.70(m, | |
| | 2H), 8.20(s, 0.4H), 8.25(s, 0.6H), 9.25(br s, | |
| | 0.6H), 9.30(d, 0.4H) | |
| 2a39 | 1.00(t, 3H), 1.78(m, 2H), 2.14(s, 3H), 3.27(s, | DMSO-d₆ |
| | 3H), 3.30(br s, 1H exchangeable), 3.65(m, 2H), | |
| | 3.98(m, 2H), 4.08(t, 2H), 6.94(m, 1H), 7.16(t, | |
| | 1H), 7.40(t, 2H), 7.71(m, 2H), 8.20(s, 0.5H), | |
| | 8.25(s, 0.5H), 9.16(br s, 0.5H), 9.24(d, 0.5H) | |
| 2a40 | 1.04(t, 3H), 1.80(m, 2H), 2.13(s, 3H), 3.30(br | DMSO-d₆ |
| | s, 1H exchangeable), 3.73(m, 2H), 3.85(m, 2H), | |
| | 4.10(t, 2H), 4.96(t, 1H), 6.92(m, 1H), 7.15(t, | |
| | 1H), 7.40(t, 2H), 7.69(m, 2H), 8.15(s, 0.5H), | |
| | 8.20(s, 0.5H), 9.15(br s, 0.5H), 9.23(d, 0.5H) | |
| 2a41 | 1.00(t, 3H), 1.79(m, 2H), 1.96(m, 2H), 2.10(s, | DMSO-d₆ |
| | 3H), 3.30(br s, 1H exchangeable), 3.65(m, 2H), | |
| | 3.88(t, 2H), 4.12(t, 2H), 4.54(t, 1H), 7.00(m, | |
| | 1H), 7.15(t, 1H), 7.42(t, 2H), 7.69(m, 2H), | |
| | 8.08(br s, 0.4H), 8.13(br s, 0.6H), 9.20(br s, | |
| | 0.6H), 9.25(d, 0.4H) | |
| 2a42 | 1.00(t, 3H), 1.80(m, 6H), 2.00(s, 3H), 2.10(s, | DMSO-d₆ |
| | 3H), 3.85(m, 2H), 4.10(m, 4H), 6.95(m, 1H), | |
| | 7.20(t, 1H), 7.40(t, 2H), 7.70(m, 2H), 8.06(s, | |
| | 0.5H), 8.15(s, 0.5H), 9.12(br s, 0.5H), 9.25 | |
| | (d, 0.5H), 11.15(br s, 1H) | |
| 2a43 | 1.00(t, 3H), 1.65(m, 2H), 1.80(m, 4H), 2.12(s, | DMSO-d₆ |
| | 3H), 3.30(br s, 1H exchangeable), 3.44(m, 2H), | |
| | 3.80(t, 2H), 4.08(t, 2H), 4.42(t, 1H), 6.96(m, | |
| | 1H), 7.20(t, 1H), 7.40(t, 2H), 7.69(m, 2H), | |
| | 8.08(s, 0.5H), 8.13(s, 0.5H), 9.20(br s, 0.5H), | |
| | 9.23(d, 0.5H) | |
| 2a44 | 1.00(t, 3H), 1.23(t, 3H), 1.80(m, 2H), 2.12(s, | DMSO-d₆ |
| | 3H), 3.30(br s, 1H exchangeable), 4.10(t, 2H), | |
| | 4.20(q, 2H), 4.60(s, 2H), 7.00(m, 1H), 7.20(t, | |
| | 1H), 7.40(t, 2H), 7.70(m, 2H), 8.20(s, 0.5H), | |
| | 8.27(s, 0.5H), 9.12(br s, 0.5H), 9.23(d, 0.5H) | |
| 2a46 | 1.00(t, 3H), 1.80(m, 2H), 2.13(s, 3H), 3.30(br | DMSO-d₆ |
| | s, 1H exchangeable), 4.10(t, 2H), 4.20(s, 2H), | |
| | 7.00(m, 1H), 7.17(t, 1H), 7.40(t, 2H), 7.50(br | |
| | s, 1H), 7.70(m, 3H), 8.04(br s, 0.5H), 8.08(s, | |
| | 0.5H), 9.09(br s, 0.5H), 9.11(d, 0.5H) | |
| 2a47 | 1.00(t, 3H), 1.18(m, 3H), 1.78(m, 2H), 2.02(m, | DMSO-d₆ |
| | 2H), 2.10(s, 3H), 2.55(t, 2H), 3.30(br s, 1H), | |
| | 3.82(t, 2H), 4.04(m, 4H), 6.98(m, 1H), 7.17(t, | |
| | 1H), 7.40(t, 2H), 7.70(m, 2H), 8.12(br s, 1H), | |
| | 9.18(br s, 0.5H), 9.22(d, 0.5H) | |
| 2a48 | 1.00(t, 3H), 1.73(m, 2H), 2.00(t, 2H), 2.10(s, | DMSO-d₆ |
| | 3H), 2.50(m, 2H), 3.30(br s, 2H exchangeable), | |
| | 3.81(t, 2H), 4.08(t, 2H), 6.96(m, 1H), 7.15(t, | |
| | 1H), 7.40(t, 2H), 7.65(m, 2H), 8.08(s, 0.4H), | |
| | 8.12(s, 0.6H), 9.15(br s, 0.6H), 9.27(d, 0.4H) | |
| 2a51 | 1.00(t, 3H), 1.16(t, 3H), 1.78(m, 6H), 2.08(s, | DMSO-d₆ |
| | 3H), 2.35(m, 2H), 3.30(br s, 1H exchangeable), | |
| | 3.80(m, 2H), 4.00(q, 2H), 4.06(t, 2H), 6.94(m, | |
| | 1H), 7.14(t, 1H), 7.40(t, 2H), 7.67(m, 2H), | |
| | 8.12(br s, 1H), 9.18(br s, 0.5H), 9.20(d, 0.5H) | |
| 2a52 | 1.00(t, 3H), 1.73(m, 6H), 2.08(s, 3H), 2.31(t, | DMSO-d₆ |
| | 2H), 3.30(br s, 2H exchangeable), 3.80(t, 2H), | |
| | 4.08(t, 2H), 6.92(m, 1H), 7.12(t, 1H), 7.40(t, | |
| | 2H), 7.65(m, 2H), 8.04(s, 0.4H), 8.12(s, 0.6H), | |
| | 9.12(br s, 0.6H), 9.21(d, 0.4H) | |
| 2a53 | 1.00(m, 6H), 1.79(m, 6H), 2.15(s, 3H), 2.16(m, | DMSO-d₆ |
| | 2H), 3.05(m, 2H), 3.80(t, 2H), 4.10(t, 2H), | |
| | 7.00(m, 1H), 7.20(t, 1H), 7.43(t, 2H), 7.73(m, | |
| | 3H), 8.15(br s, 1H), 9.15(m, 1H), 11.15(br s, 1H) | |
| 2a54 | 1.00(t, 3H), 1.78(m, 6H), 2.08(s, 3H), 2.40(t, | DMSO-d₆ |
| | 2H), 3.40(m, 4H), 3.50(m, 4H), 3.80(t, 2H), | |
| | 4.08(t, 2H), 6.94(m, 1H), 7.14(t, 1H), 7.40(t, | |
| | 2H), 7.70(m, 2H), 8.05(s, 0.5H), 8.12(s, 0.5H), | |
| | 9.17(br s, 0.5H), 9.20(d, 0.5H), 11.20(br s, 1H) | |
| 2a64 | 1.00(t, 3H), 1.80(m, 2H), 2.12(s, 3H), 2.60(m, | DMSO-d₆ |
| | 1H), 2.80(m, 1H), 3.30(br s, 2H exchangeable), | |
| | 3.73(d, 2H), 4.08(t, 2H), 6.23(m, 1H), 5.23(br | |
| | s, 1H), 7.00(d, 1H), 7.20(t, 1H), 7.40(t, 2H), | |
| | 7.70(d, 2H), 8.20(s, 1H), 9.20(br s, 1H) | |
| 2a65 | 1.00(t, 3H), 1.35(s, 3H), 1.42(s, 3H), 1.80(m, | DMSO-d₆ |
| | 2H), 2.13(s, 3H), 3.30(br s, 2H exchangeable), | |
| | 4.00(m, 2H), 4.12(t, 2H), 4.42(s, 2H), 7.00(m, | |
| | 1H), 7.16(t, 1H), 7.40(m, 2H), 7.70(m, 2H), | |
| | 8.20(s, 0.5H), 8.23(s, 0.5H), 8.12(br s, 0.5H), | |
| | 9.20(d, 0.5H) | |
| 2a67 | 1.00(t, 3H), 1.73(m, 2H), 2.12(s, 3H), 3.46(m, | DMSO-d₆ |
| | 2H), 3.73(m, 1H), 3.81(m, 2H), 4.08(t, 2H), | |
| | 4.62(t, 1H), 5.00(d, 1H), 6.92(m, 1H), 7.15(t, | |
| | 1H), 7.40(t, 2H), 7.65(d, 2H), 8.15(s, 0.5H), | |
| | 8.23(s, 0.5H), 9.12(br s, 0.5H), 9.21(d, 0.5H), | |
| | 11.10(br s, 1H) | |
| 2a68 | 1.00(t, 3H), 1.73(m, 2H), 2.12(s, 3H), 3.46(m, | DMSO-d₆ |
| | 2H), 3.73(m, 1H), 3.81(m, 2H), 4.08(t, 2H), | |
| | 4.62(t, 1H), 5.00(d, 1H), 6.92(m, 1H), 7.15(t, | |
| | 1H), 7.40(t, 2H), 7.65(d, 2H), 8.15(s, 0.5H), | |
| | 8.23(s, 0.5H), 9.12(br s, 0.5H), 9.21(d, 0.5H), | |
| | 11.10(br s, 1H) | |
| 2a69 | 1.00(t, 3H), 1.73(m, 2H), 2.12(s, 3H), 3.46(m, | DMSO-d₆ |
| | 2H), 3.73(m, 1H), 3.81(m, 2H), 4.08(t, 2H), | |
| | 4.62(t, 1H), 5.00(d, 1H), 6.92(m, 1H), 7.15(t, | |
| | 1H), 7.40(t, 2H), 7.65(d, 2H), 8.15(s, 0.5H), | |
| | 8.23(s, 0.5H), 9.12(br s, 0.5H), 9.21(d, 0.5H), | |
| | 11.10(br s, 1H) | |
| 2a70 | 1.00(t, 3H), 1.10(m, 3H), 1.80(m, 2H), 2.10(s, | DMSO-d₆ |
| | 3H), 3.70(m, 1H), 3.95(m, 1H), 4.10(m, 4H), | |
| | 4.20(m, 1H), 4.25(m, 1H), 5.25(m, 2H), 7.00(m, | |
| | 1H), 7.20(t, 1H), 7.40(t, 2H), 7.70(m, 2H), | |
| | 8.20(br s, 1H), 9.20(m, 1H), 11.10(br s, 1H) | |
| 2a71 | 1.00(t, 3H), 1.80(m, 2H), 2.16(s, 3H), 3.30(br | DMSO-d₆ |
| | s, 2H exchangeable), 3.73(m, 1H), 3.96(m, 1H), | |
| | 4.12(t, 2H), 4.23(s, 2H), 4.95(br s, 1H), | |
| | 5.20(br s, 1H), 7.00(m, 1H), 7.20(t, 1H), | |
| | 7.40(t, 2H), 7.70(m, 2H), 8.15(s, 0.5H), | |
| | 8.20(s, 0.5H), 9.20(br s, 0.5H), 9.21(d, 0.5H) | |
| 2b1a | 2.08(s, 3H), 2.50(s, 3H), 6.80(d, 1H), 7.50(d, | DMSO-d₆ |
| | 1H), 10.0(s, 1H), 10.35(br s, 1H) | |
| 2b1b | 1.08(t, 3H), 1.85(m, 2H), 2.20(s, 3H), 2.60(s, | CDCl₃ |
| | 3H), 4.00(t, 2H), 6.80(d, 1H), 7.62(d, 1H), | |
| | 10.12(s, 1H) | |
| 2b15c | 1.08(t, 3H), 1.77(m, 6H), 2.65(m, 2H), 3.15(m, | CDCl₃ |
| | 2H), 4.00(t, 2H), 6.73(d, 1H), 7.58(d, 1H), | |
| | 10.08(s, 1H) | |
| 2b16b | 1.04(t, 3H), 1.60(m, 2H), 2.40(s, 3H), 2.50(m, | DMSO-d₆ |
| | 2H), 2.54(s, 3H), 7.37(d, 1H), 7.60(d, 1H), | |
| | 10.25(s, 1H) | |
| 2b16c | 0.85(t, 3H), 1.30(m, 4H), 1.50(m, 2H), 2.20(s, | DMSO-d₆ |
| | 3H), 2.54(s, 3H), 2.65(m, 2H), 7.20(d, 1H), | |
| | 7.54(d, 1H) 10.20(s, 1H) | |
| 2b17 | 1.20(t, 3H), 2.00(m, 2H), 4.20(t, 2H), 6.90(d, | CDCl₃ |
| | 1H), 7.56(dd, 1H), 7.70(dd, 1H), 7.87(d, 1H), | |
| | 8.35(d, 1H), 9.27(d, 1H), 10.20(s, 1H) | |
| 2b18c | 1.08(t, 3H), 1.85(m, 2H), 2.08(m, 2H), 2.85(t, | CDCl₃ |
| | 2H), 2.96(t, 2H), 4.00(t, 2H), 6.65(d, 1H), | |
| | 6.85(d, 1H), 7.10(dd, 1H) | |
| 2b18e | 1.05(t, 3H), 1.81(m, 2H), 2.15(m, 2H), 2.85(t, | CDCl₃ |
| | 2H), 3.27(t, 2H), 4.00(t, 2H), 6.77(d, 1H), | |
| | 7.60(d, 1H), 10.00(s, 1H) | |
| 2b19a | 1.10(t, 3H), 1.95(m, 2H), 4.05(t, 2H), 6.95(d, | CDCl₃ |
| | 1H), 7.45(m, 2H), 8.00(d, 1H), 8.46(d, 1H), | |
| | 9.15(s, 1H) | |
| 2b19c | 1.20(t, 3H), 2.04(m, 2H), 4.20(t, 2H), 7.04(d, | CDCl₃ |
| | 1H), 8.00(d, 1H), 8.08(d, 1H), 8.65(d, 1H), | |
| | 10.20(s, 1H), 10.57(s, 1H) | |
| 2b20b | 1.04(t, 3H), 1.85(m, 2H), 4.00(t, 2H), 6.73(d, | CDCl₃ |
| | 1H), 7.20(d, 1H), 7.42(m, 2H), 8.40(d, 1H), | |
| | 9.50(s, 1H) | |
| 2b21c | 1.04(t, 3H), 1.50(s, 9H), 1.80(m, 2H), 2.75(t, | CDCl₃ |
| | 2H), 3.60(t, 2H), 3.90(t, 2H), 4.50(s, 2H), | |
| | 6.60(d, 1H), 7.31(d, 1H) | |
| 2b21d | 1.06(t, 3H), 1.50(s, 9H), 1.85(m, 2H), 2.80(t, | CDCl₃ |
| | 2H), 3.62(t, 2H), 4.00(t, 2H), 5.00(s, 2H), | |
| | 6.80(d, 1H), 7.62(d, 1H), 10.00(s, 1H) | |
| 2b23 | 1.00(t, 3H), 1.06(t, 3H), 1.82(m, 4H), 3.92(t, | CDCl₃ |
| | 2H), 4.00(t, 2H), 6.40(s, 1H), 6.50(d, 1H), | |
| | 7.80(d, 1H), 10.30(s, 1H) | |
| 2b25 | 1.06(t, 3H), 1.85(m, 2H), 2.12(s, 3H), 4.00(t, | CDCl₃ |
| | 2H), 6.50(d, 1H), 7.30(d, 1H), 9.65(s, 1H), | |
| | 11.40(s, 1H) | |
| 2b28 | 1.08(t, 3H), 1.86(m, 2H), 2.20(s, 3H), 3.45(s, | CDCl₃ |
| | 3H), 3.75(m, 2H), 4.00(t, 2H), 4.08(m, 2H), | |
| | 6.71(d, 1H), 7.71(s, 1H), 10.25(s, 1H) | |
| 2b29b | 1.10(t, 3H), 1.65(br s, 1H), 1.87(m, 2H), | CDCl₃ |
| | 2.20(s, 3H), 4.00(m, 6H), 6.69(d, 1H), 7.62(d, | |
| | 1H), 10.06(s, 1H) | |
| 2b32 | 1.08(t, 3H), 1.60(br s, 1H), 1.80(m, 4H), | CDCl₃ |
| | 1.92(m, 2H), 2.15(s, 3H), 3.73(t, 2H), 3.92(t, | |
| | 2H), 4.00(t, 2H), 6.65(d, 1H), 7.65(d, 1H), | |
| | 10.20(s, 1H) | |
| 2b35 | 1.04(t, 3H), 1.80(m, 2H), 2.12(s, 3H), 4.00(t, | CDCl₃ |
| | 2H), 4.31(s, 2H), 6.60(br s, 1H), 6.70(d, 1H), | |
| | 7.40(br s, 1H), 7.60(d, 1H), 9.90(s, 1H) | |
| 2b40 | 1.08(t, 3H), 1.27(t, 3H), 1.86(m, 6H), 2.16(s, | CDCl₃ |
| | 3H), 2.39(m, 2H), 3.90(m, 2H), 4.00(t, 2H), | |
| | 4.12(q, 2H), 6.67(d, 1H), 7.67(d, 1H), 10.20(s, | |
| | 1H) | |
| 2b41 | 1.00(t, 3H), 1.73(m, 6H), 2.08(s, 3H), 2.27(t, | DMSO-d₆ |
| | 2H), 3.85(t, 2H), 4.04(t, 2H), 6.88(d, 1H), | |
| | 7.58(d, 1H), 10.04(s, 1H), 12.00(br s, 1H) | |
| 2b54 | 1.00(t, 3H), 1.80(m, 3H), 2.10(s, 3H), 2.70(m, | CDCl₃ |
| | 2H), 3.85(m, 1H), 3.95(m, 3H), 4.35(m, 1H), | |
| | 6.70(d, 1H), 7.50(s, 1H), 9.80(s, 1H) | |
| 11b | 2.20(s, 3H), 3.46(s, 2H), 7.31(m, 4H), 11.0(br | DMSO-d₆ |
| | s, 1H) | |
| 12a1 | 1.00(t, 3H), 1.73(m, 2H), 2.13(s, 3H), 2.38(s, | DMSO-d₆ |
| | 3H), 4.04(t, 2H), 6.90(d, 1H), 8.00(s, 1H), | |
| | 8.85(d, 1H), 10.29(br s, 2H) | |
| 12a3 | 1.00(t, 3H), 1.69(m, 6H), 2.54(m, 2H), 2.85(m, | DMSO-d₆ |
| | 2H), 4.00(t, 2H), 6.85(d, 1H), 7.96(s, 1H), | |
| | 8.92(d, 1H), 10.27(br s, 2H) | |

### Example 16

### P2Y₁₂ Receptor Binding Assay

Chinese Hamster Ovary (CHO) cells with recombinant expression of the human P2Y₁₂ receptor were cultured in 24 well cell-culture plates. Cells were washed three times with binding buffer (50 mM Tris pH 7.4, 100 mM NaCl, 1 mM EDTA, 0.5% BSA). The cells were then incubated with 0.5 ml per well binding buffer containing tritium-labeled 2-methyl-thio-adenosine 5′-diphosphate (2-methyl-S-ADP) (between 100'000 and 300'000 dpm per well) and various concentrations of test compounds. After incubation at room temperature for 2 hours, cells were washed three times with binding buffer. Then, cells were solubilized by addition of 0.5 ml solubilization buffer (SDS, NaOH, EDTA). The content of each well was then transferred into beta-counter vials and 2.0 ml of Ultima Gold Scintillation liquid was added. After quantification of the cell-associated signal, extent of inhibition was calculated relative to maximal possible inhibition demonstrated by addition of excess of cold 2-methyl-S-ADP.

### Example 17

### Test for Antagonist Binding to the Platelet ADP Receptor P2Y₁₂

The test is conducted as described hereinabove. Compounds of formula I showed in this test IC₅₀ values ranging between about 0.001 and about 10 μM. Preferred compounds showed IC₅₀ values below 1 μM, particularly preferred compounds showed IC₅₀ values below 0.1 μM and still more preferred compounds showed IC₅₀ values below 0.01 μM. Exemplary IC₅₀ values are given below.

| Compound of Example | IC₅₀ (μM) |
|---|---|
| 1 | 0.024 |
| 2a7 | 0.19 |
| 2a12 | 0.47 |
| 2a25 | 0.03 |
| 2a30 | 0.01 |
| 2a35 | 0.37 |
| 2a39 | 0.015 |
| 2a43 | 0.005 |
| 2a52 | 0.0016 |
| 2a63 | 0.001 |
| 2a64 | 0.0008 |
| 2a71 | 0.003 |
| 4a2 | 0.14 |
| 8a | 0.083 |
| 12a3 | 0.055 |
| 12a9 | 0.04 |
| 15 | 9.6 |

### Example 18

### ADP Induced Platelet Aggregation

### 18a) Preparation of Platelet-Rich Plasma (PRP)

After obtaining informed consent, blood was obtained by vein puncture from healthy volunteers using trisodium citrate, at 108 mM final concentration, as the anticoagulant. Platelet-rich plasma (PRP) was separated by centrifugation at 20° C. for 10 minutes at 160 g. Part of the blood was centrifuged for 10 minutes at 5000 g to yield platelet poor plasma (PPP).

### 18b) ADP induced Platelet Aggregation

Platelet aggregation was measured in a Chronolog lumiaggregometer with stirring (900 rpm) at 37° C. PRP was placed into the cuvette and allowed to equilibrate at 37° C. for two min. In a first phase, the ADP concentration to give optimal extent of aggregation was determined for the PRP of each donor. In a second phase, PRP was incubated with inhibitors for 2 min at 37° C. prior to the addition of the agonist ADP at 1-5 μM final concentration.

The change in light absorbance, indicative of ongoing aggregation, was monitored during 5 min. The extent of platelet aggregation was calculated relative to light absorbance of PRP (not aggregated) and PPP (full aggregation).

### Example 19

### Functional Assay (FLIPR)

Chinese Hamster Ovary (CHO) cells stably expressing the human P2Y₁₂ receptor under the control of the cytomegalovirus promoter in the expression vector pcDNA3 (Invitrogen) were grown to near confluency in Ham's F-12 medium supplemented with 10% fetal calf serum (both Bioconcept, Switzerland) under standard mammalian cell culture conditions (37° C. and 5% carbon dioxide). Cells were treated with 0.02% EDTA in phosphate buffer saline (PBS, Gibco) for 10 min, detached by tapping, and collected by centrifugation for five minutes at 200 g, all at room temperature. They were incubated one hour stirring at 37° C. and 5% CO₂ with 4 μM Fluo-3, 0.04% Pluronic F-127 (both Molecular Probes), 5 mM probenecid (Sigma), 20 mM HEPES (Gibco) in assay buffer (equal parts of Hank's BSS (HBSS, Bioconcept) and Ham's F-12). They were then washed with and resuspended in assay buffer. 50,000 cells in 60 μl were transferred to each well of a 384-well FLIPR assay plate (Greiner) and sedimented by centrifugation. A FLIPR384 instrument (Molecular Devices) was operated following the manufacturer's standard instructions, adding 10 μl of compound dissolved at 10 mM in DMSO and diluted prior to the experiment in assay buffer to obtain the desired final concentration. 10 μl of ADP (Sigma) solution in assay buffer supplemented with bovine serum albumin (fatty acid content <0.02%, Sigma) was then added to obtain a final concentration of 3 μM and 0.1%, respectively. Fluorescence emission was recorded during both additions.

### Example 20

### Gelatin Solution

A sterile-filtered aqueous solution, with 2% cyclodextrins as solubilisers, of one of the compounds of formula (III) mentioned in the preceding Examples (e.g. Example 8a) as active ingredient, is so mixed under aseptic conditions, with heating, with a sterile gelatin solution containing phenol as preservative, that 1.0 ml of solution has the following composition:

| | | |
|---|---|---|
| active ingredient | 3 | mg |
| gelatin | 150.0 | mg |
| phenol | 4.7 | mg |
| dist. water with 20% cyclodextrins as solubilisers | 1.0 | ml |

### Example 21

### Sterile Dry Substance for Injection

5 mg of one of the compounds of formula (III) mentioned in the preceding Examples (e.g. Example 12a1) as active ingredient are dissolved in 1 ml of an aqueous solution with 20 mg of mannitol and 20% cyclodextrins as solubilisers. The solution is sterile-filtered and introduced under aseptic conditions into a 2 ml ampoule, deep-frozen and lyophilized. Before use, the lyophilisate is dissolved in 1 ml of distilled water or 1 ml of physiological saline solution. The solution is administered intramuscularly or intravenously. This formulation can also be introduced into a twin-chambered injection ampoule.

### Example 22

### Film-Coated Tablets

The following ingredients are used for the preparation of 10,000 tablets each containing 100 mg of active ingredient:

| | | |
|---|---|---|
| active ingredient | 1000 | g |
| corn starch | 680 | g |
| colloidal silica | 200 | g |
| magnesium stearate | 20 | g |
| stearic acid | 50 | g |
| sodium carboxymethyl starch | 250 | g |

| | |
|---|---|
| water | quantum satis |

A mixture of one of the compounds of formula (III) mentioned in the preceding Examples (e.g. Example 4a2) as active ingredient, 50 g of corn starch and the colloidal silica is processed with a starch paste, made from 250 g of corn starch and 2.2 kg of demineralised water, to form a moist mass. This is forced through a sieve having a mesh size of 3 mm, and dried at 45% C for 30 mm in a fluidized bed drier. The dry granulates are pressed through a sieve having a mesh size of 1 mm, mixed with a pre-sieved mixture (1 mm sieve) of 330 g of corn starch, the magnesium stearate, the stearic acid and the sodium carboxymethyl starch, and compressed to form slightly biconvex tablets.

### Example 23

### Soft Capsules

5000 soft gelatin capsules, each comprising as active ingredient 0.05 g of one of the compounds of formula (III) mentioned in the preceding Examples are prepared as follows:

| | | |
|---|---|---|
| active ingredient | 250 | g |
| lauroglycol ® | 2 | liters |

The pulverized active ingredient is suspenden in Lauroglykol® (propylene glycol laureate, Glattefossé S. A., Saint Priest, France) and ground in a wet pulverizer to produce a particle size of about 1 to 3 μm. 0.42 g portions of the mixture are then introduced into soft gelatin capsules using a capsule-filling machine.

1. A method for preventing or treating a disease or a condition associated with platelet aggregation, said method comprising administering to a mammal in need thereof at least one compound selected from the group consisting of:
a pyrazolidinedione derivative of formula (I), 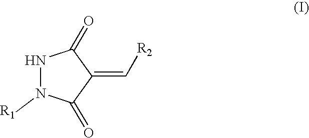
wherein
R₁ is hydrogen, optionally substituted alkyl, cycloalkyl, aryl, arylalkyl, heteroaryl, heteroarylalkyl or alkanoyl; and
R₂ is aryl or heteroaryl,
tautomers thereof,
geometric isomers thereof or tautomers of these geometric isomers; and
a mixture thereof: including mixtures of individual compounds of formula (I), or tautomers thereof, and their geometric isomers, or tautomers thereof; or
a pharmaceutically acceptable acid addition salts of compounds which are basic salt of said compound which is basic; or
a pharmaceutically acceptable salt of said compound containing an acidic group with a base; or
a pharmaceutically acceptable ester of said compound containing a hydroxy or carboxy group; or
a prodrug of said compound in which a prodrug forming group is present; or a hydrate or solvate thereof.

2. The method according to claim 1, wherein R₁ is other than alkanoyl.

3. The method according to claim 1, wherein the disease or condition is thrombosis.

4. The method according to claim 1, wherein R₁ is hydrogen, alkyl, aryl, heteroaryl or alkanoyl.

5. The method according to claim 4, wherein R₁ is hydrogen, alkyl, phenyl, bromophenyl, chlorophenyl, fluorophenyl, methylphenyl, methoxyphenyl, cyanophenyl, alkoxycarbonylphenyl, pyridinyl or alkanoyl.

6. The method according to claim 5, wherein R₁ is hydrogen, methyl, phenyl, 2-pyridinyl, 4-pyridinyl, 2-methylphenyl, 4-methylphenyl, 4-methoxyphenyl, 2-chlorophenyl, 4-fluorophenyl, 4-bromophenyl, 4-cyanophenyl, 4-ethoxycarbonylphenyl or acetyl.

7. The method according to claim 1, wherein R₂ is naphthalenyl, thienyl or pyridyl.

8. The method according to claim 7, wherein R₂ is naphthalen-2-yl, pyridin-3-yl or thiophen-3-yl.

9. The method according to claim 1, wherein said compound is at least one selected from the group consisting of:
a pyrazolidinedione derivative of formula (III), 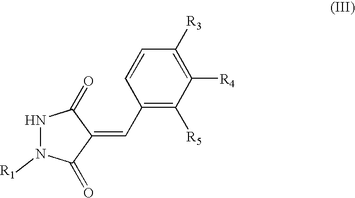
geometric isomers thereof, tautomers thereof and mixtures thereof;
or a salt, ester or prodrug of said compound,
wherein
R₃ is hydrogen, alkyl, alkenyl, alkynyl, alkoxy, hydroxyalkoxy, alkoxyalkoxy, alkenyloxy, cycloalkoxy, cycloalkylalkoxy or alkylsulfonyloxy;
R₄ is hydrogen, halogen, hydroxy, alkyl or alkoxy; and
R₅ is hydrogen, halogen, hydroxy, alkyl, alkoxy, alkoxyalkoxy, hydroxyalkoxy, dihydroxyalkoxy, alkanoyloxyalkoxy, carboxyalkoxy, carboxy-hydroxyalkoxy, carboxy-dihydroxyalkoxy, alkoxycarbonylalkoxy, alkoxycarbonyl-hydroxyalkoxy, alkoxycarbonyl-dihydroxyalkoxy, carbamoylalkoxy, N-alkylcarbamoylalkoxy, N,N-dialkyaminolalkoxy, morpholin-4-ylalkoxy, piperidin-1-ylalkoxy, morpholin-4-ylcarbonylalkoxy, 2,2-dialkyl[1,3]dioxolan-4-ylalkoxy or 2,2-dialkyl-4-carboxy[1,3]dioxolan-5-ylalkoxy; or
R₄ and R₅, together with the phenyl ring to which they are attached, form a fused, optionally substituted carbocyclic or heterocyclic ring system.

10. The method according to claim 9,
wherein
R₁ is other than alkanoyl;
R₃ is hydrogen, alkyl, alkenyl, alkynyl, alkoxy, hydroxyalkoxy, alkoxyalkoxy, alkenyloxy, cycloalkoxy or cycloalkylalkoxy; and
R₄ and R₅, each independently of the other, are hydrogen, halogen, hydroxy, alkyl or alkoxy; or
R₄ and R₅, together with the phenyl ring to which they are attached, form a fused, optionally substituted carbocyclic or heterocyclic ring system.

11. The method according to claim 9,
wherein
R₃ is alkyl, alkenyl, alkynyl, alkoxy, cycloalkoxy, cycloalkylalkoxy, hydroxyalkoxy or alkoxyalkoxy; and
R₄ and R₅ both are hydrogen, or R₄ is halogen, alkyl or alkoxy and R₅ is hydrogen, or R₄ and R₅ each independently are alkyl or alkoxy.

12. The method according to claim 11
wherein
R₃ is methyl, ethyl, propyl, iso-propyl, butyl, iso-butyl, tert-butyl, pentyl, hexyl, but-1-enyl, pent-1-enyl, but-1-ynyl, pent-1-ynyl, methoxy, ethoxy, propoxy, butoxy, iso-butoxy, 3-methyl-butoxy, pentyloxy, cyclopentyloxy, hexyloxy, cyclopropylmethoxy, cyclobutylmethoxy, 2-hydroxy-ethoxy, or 2-methoxy-ethoxy, and
R₄ and R₅ both are hydrogen or R₄ is chloro, bromo, methyl or methoxy and R₅ is hydrogen, or R₄ and R₅ each independently are methyl or methoxy.

13. The method according to claim 9,
wherein
R₃ is hydrogen or alkoxy; and
R₄ and R₅ together with the phenyl ring to which they are attached, form an optionally substituted naphthalene, tetrahydronaphthalene, indane, 1H-indene, isoquinoline, dihydro-benzo[1,4]dioxine or benzo[1,3]dioxole moiety.

14. The method according to claim 13,
wherein
R₃ is propoxy; and
R₄ and R₅ together with the phenyl ring to which they are attached, form a naphthalene-1-yl, indan-4-yl, isoquinolin-5-yl, isoquinolin-8-yl, 1,2,3,4-tetrahydroisoquinolin-8-yl, 2-alkoxycarbonyl-1,2,3,4-tetrahydroisoquinolin-8-yl or 5,6,7,8-tetrahydronaphthalen-1-yl moiety.

15. The method according to claim 9,
wherein
R₃, R₄ and R₅ each are hydrogen; or
R₃ and R₅ each are hydrogen and R₄ is methoxy; or R₃ and R₄ each are hydrogen and R₅ is methoxy; or
R₄ and R₅ each are hydrogen and R₃ is tert-butyl, ethoxy, propoxy or butoxy; or
R₃ is hydrogen, R₄ is methoxy, and R₅ is hydroxy; or
R₄ is hydrogen, R₃ is methoxy or propoxy and R₅ is methoxy or propoxy; or
R₅ is hydrogen, R₃ is methoxy, ethoxy, propoxy, butyloxy, iso-butyloxy, pentyloxy, hexyloxy, 3-methylbutoxy, 2-hydroxyethoxy, 2-methoxyethoxy, cyclopropylmethoxy or cyclobutylmethoxy and R₄ is methyl, methoxy, chloro or bromo.

16. The method according to claim 9,
wherein
R₃ is methoxy, propoxy, cyclopentyloxy, pent-1-ynyl or ethanesulfonyloxy;
R₄ is methyl;
R₅ is hydroxy, methyl, pentyl, methoxy, propoxy, 2-methoxyethoxy, 2-hydroxyethoxy, 3-hydroxypropoxy, 4-hydroxybutoxy, 2,3-dihydroxypropoxy, 4-acetoxybutoxy, carboxymethoxy, 3-carboxypropoxy, 4-carboxybutoxy, 3-carboxy-2-hydroxypropoxy, 3-carboxy-2,3-dihydroxypropoxy, ethoxycarbonylmethoxy, 3-ethoxycarbonylpropoxy, 4-ethoxycarbonylbutoxy, 3-ethoxycarbonyl-2-hydroxypropoxy, 3-ethoxycarbonyl-2,3-dihydroxypropoxy, carbamoylmethoxy 3-N-ethylcarbamoylpropoxy, 4-N-ethylcarbamoylbutoxy, 2-N,N-dimethylaminoethoxy, 3-N,N-dmethylaminopropoxy, 2-(morpholin-4-yl)-ethyoxy, 2-(piperidin-1-yl)-ethoxy, 3-(morpholin-4-yl)-carbonylpropoxy, 4-(morpholin-4-yl)-carbonylbutoxy, 2,2-dimethyl[1,3]dioxolan-4-ylmethoxy or 2,2-dimethyl-4-carboxy[1,3]dioxolan-5-ylmethoxy; or
R₄ and R₅, together with the phenyl ring to which they are attached, form a naphthalen-1-yl, 5,6,7,8-tetrahydronaphthalen-1-yl, indan-4-yl, 1,2,3,4,-tetraisoquinolin-8-yl or 2-tert-butoxycarbonyl-1,2,3,4,-tetraisoquinolin-8-yl moiety.

17. The method according to claim 1, wherein the compound is selected from the group consisting of:
4-(2,3-dimethyl-4-propoxy-benzylidene)-1-phenyl-pyrazolidine-3,5-dione;
4-(4-methoxy-3-methyl-benzylidene)-1-phenyl-pyrazolidine-3,5-dione;
4-(4-ethoxy-benzylidene)-1-phenyl-pyrazolidine-3,5-dione;
4-(4-ethoxy-3-methoxy-benzylidene)-1-phenyl-pyrazolidine-3,5-dione;
4-(2,4-dimethoxy-benzylidene)-1-phenyl-pyrazolidine-3,5-dione;
4-naphthalen-2-ylmethylene-1-phenyl-pyrazolidine-3,5-dione;
4-(4-tert-butyl-benzylidene)-1-phenyl-pyrazolidine-3,5-dione;
4-(2,3-dimethyl-4-methoxybenzylidene)-1-phenyl-pyrazolidine-3,5-dione;
4-(2,4-dimethoxy-3-methyl-benzylidene)-1-phenyl-pyrazolidine-3,5-dione;
4-(3-bromo-4-methoxy-benzylidene)-1-phenyl-pyrazolidine-3,5-dione;
1-phenyl-4-(4-propoxy-benzylidene)-pyrazolidine-3,5-dione;
4-(4-butoxy-benzylidene)-1-phenyl-pyrazolidine-3,5-dione;
4-(4-ethoxy-3-methyl-benzylidene)-1-phenyl-pyrazolidine-3,5-dione;
4-(3-methyl-4-propoxy-benzylidene)-1-phenyl-pyrazolidine-3,5-dione;
4-(4-butoxy-3-methyl-benzylidene)-1-phenyl-pyrazolidine-3,5-dione;
4-(4-hexyloxy-3-methyl-benzylidene)-1-phenyl-pyrazolidine-3,5-dione;
4-(3-methyl-4-pentyloxy-benzylidene)-1-phenyl-pyrazolidine-3,5-dione;
4-(4-cyclobutylmethoxy-3-methyl-benzylidene)-1-phenyl-pyrazolidine-3,5-dione;
4-[3-methyl-4-(3-methyl-butoxy)-benzylidene]-1-phenyl-pyrazolidine-3,5-dione;
4-(4-isobutoxy-3-methyl-benzylidene)-1-phenyl-pyrazolidine-3,5-dione;
4-[4-(2-methoxy-ethoxy)-3-methyl-benzylidene]-1-phenyl-pyrazolidine-3,5-dione;
4-(3-chloro-4-propoxy-benzylidene)-1-phenyl-pyrazolidine-3,5-dione;
4-[4-(2-hydroxy-ethoxy)-3-methyl-benzylidene]-1-phenyl-pyrazolidine-3,5-dione;
4-(4-cyclopropylmethoxy-3-methyl-benzylidene)-1-phenyl-pyrazolidine-3,5-dione;
4-(4-cyclopentyloxy-2,3-dimethyl-benzylidene)-1-phenyl-pyrazolidine-3,5-dione;
1-phenyl-4-(4-propoxy-5,6,7,8-tetrahydro-naphthalen-1-ylmethylene)-pyrazolidine-3,5-dione;
4-(2,3-dimethyl-4-pent-1-ynyl-benzylidene)-1-phenyl-pyrazolidine-3,5-dione;
4-(2,3-dimethyl-4-pentyl-benzylidene)-1-phenyl-pyrazolidine-3,5-dione;
1-phenyl-4-(4-propoxy-naphthalen-1-ylmethylene)-pyrazolidine-3,5-dione;
1-phenyl-4-[1-(7-propoxy-indan-4-yl)-methylidene]-pyrazolidine-3,5-dione;
1-phenyl-4-[1-(5-propoxy-isoquinolin-8-yl)-methylidene]-pyrazolidine-3,5-dione;
1-phenyl-4-[1-(8-propoxy-isoquinolin-5-yl)-methylidene]-pyrazolidine-3,5-dione;
4-[1-(2-tert-butoxycarbonyl-5-propoxy-1,2,3,4-tetrahydro-isoquinolin-8-yl)-methylidene]-1-phenyl-pyrazolidine-3,5-dione;
4-(2,3-dimethyl-4-ethanesulfonyloxy-benzylidene)-1-phenyl-pyrazolidine-3,5-dione;
4-[1-(2,4-dipropoxy-phenyl)-methylidene]-1-phenyl-pyrazolidine-3,5-dione;
4-[1-(2,6-dipropoxy-pyridin-3-yl)-methylidene]-1-phenyl-pyrazolidine-3,5-dione;
4-[1-(2-hydroxy-3-methyl-4-propoxy-phenyl)-methylidene]-1-phenyl-pyrazolidine-3,5-dione;
4-[1-(2-methoxy-3-methyl-4-propoxy-phenyl)-methylidene]-1-phenyl-pyrazolidine-3,5-dione;
4-[1-(3-methyl-2,4-dipropoxy-phenyl)-methylidene]-1-phenyl-pyrazolidine-3,5-dione;
4-[1-[2-(2-methoxy-ethoxy)-3-methyl-4-propoxy-phenyl]-methylidene]-1-phenyl-pyrazolidine-3,5-dione;
4-[1-[2-(2-hydroxy-ethoxy)-3-methyl-4-propoxy-phenyl]-methylidene]-1-phenyl-pyrazolidine-3,5-dione;
4-[1-[2-(3-hydroxy-propoxy)-3-methyl-4-propoxy-phenyl]-methylidene]-1-phenyl-pyrazolidine-3,5-dione;
4-[1-[2-(4-acetoxy-butoxy)-3-methyl-4-propoxy-phenyl]-methylidene]-1-phenyl-pyrazolidine-3,5-dione;
4-[1-[2-(4-hydroxy-butoxy)-3-methyl-4-propoxy-phenyl]-methylidene]-1-phenyl-pyrazolidine-3,5-dione;
4-[1-[2-(ethoxycarbonyl-methoxy)-3-methyl-4-propoxy-phenyl]-methylidene]-1-phenyl-pyrazolidine-3,5-dione;
4-[1-[2-(carboxy-methoxy)-3-methyl-4-propoxy-phenyl]-methylidene]-1-phenyl-pyrazolidine-3,5-dione;
4-[1-[2-(2-amino-2-oxo-ethyloxy)-3-methyl-4-propoxy-phenyl]-methylidene]-1-phenyl-pyrazolidine-3,5-dione;
4-[1-[2-(3-ethoxycarbonyl-propoxy)-3-methyl-4-propoxy-phenyl]-methylidene]-1-phenyl-pyrazolidine-3,5-dione;
4-[1-[2-(3-carboxy-propoxy)-3-methyl-4-propoxy-phenyl]-methylidene]-1-phenyl-pyrazolidine-3,5-dione;
4-[1-[2-(4-ethylamino-4-oxo-butoxy)-3-methyl-4-propoxy-phenyl]-methylidene]-1-phenyl-pyrazolidine-3,5-dione;
4-[1-[3-methyl-2-(4-morpholin-4-yl-4-oxo-butoxy)-4-propoxy-phenyl]-methylidene]-1-phenyl-pyrazolidine-3,5-dione;
4-[1-[2-(4-ethoxycarbonyl-butoxy)-3-methyl-4-propoxy-phenyl]-methylidene]-1-phenyl-pyrazolidine-3,5-dione;
4-[1-[2-(4-carboxy-butyloxy)-3-methyl-4-propoxy-phenyl]-methylidene]-1-phenyl-pyrazolidine-3,5-dione;
4-[1-[2-(5-ethylamino-5-oxo-pentyloxy)-3-methyl-4-propoxy-phenyl]-methylidene]-1-phenyl-pyrazolidine-3,5-dione;
4-[1-[3-methyl-2-(5-morpholin-4-yl-5-oxo-pentyloxy)-4-propoxy-phenyl]-methylidene]-1-phenyl-pyrazolidine-3,5-dione;
4-[1-[2-(2-dimethylamino-ethoxy)-3-methyl-4-propoxy-phenyl]-methylidene]-1-phenyl-pyrazolidine-3,5-dione and its hydrochloride salt;
4-[1-[3-methyl-2-(2-morpholin-4-yl-ethoxy)-4-propoxy-phenyl]-methylidene]-1-phenyl-pyrazolidine-3,5-dione and its hydrochloride salt;
4-[1-[3-methyl-2-(2-piperidin-1-yl-ethoxy)-4-propoxy-phenyl]-methylidene]-1-phenyl-pyrazolidine-3,5-dione and its hydrochloride salt;
4-[1-[2-(3-dimethylamino-propoxy)-3-methyl-4-propoxy-phenyl]-methylidene]-1-phenyl-pyrazolidine-3,5-dione and its hydrochloride salt;
4-[1-[2-(2,2-dimethyl-[1,3]dioxolan-4-ylmethoxy)-3-methyl-4-propoxy-phenyl]-methylidene]-1-phenyl-pyrazolidine-3,5-dione;
4-[1-[2-((4R)-2,2-dimethyl-[1,3]dioxolan-4-ylmethoxy)-3-methyl-4-propoxy-phenyl]-methylidene]-1-phenyl-pyrazolidine-3,5-dione;
4-[1-[2-((4S)-2,2-dimethyl-[1,3]dioxolan-4-ylmethoxy)-3-methyl-4-propoxy-phenyl]-methylidene]-1-phenyl-pyrazolidine-3,5-dione;
4-[1-[2-((2R)-3-ethoxycarbonyl-2-hydroxy-propoxy)-3-methyl-4-propoxy-phenyl]-methylidene]-1-phenyl-pyrazolidine-3,5-dione;
4-[1-[2-((2R)-3-carboxy-2-hydroxy-propoxy)-3-methyl-4-propoxy-phenyl]-methylidene]-1-phenyl-pyrazolidine-3,5-dione;
4-[1-[2-((2S)-3-carboxy-2-hydroxy-propoxy)-3-methyl-4-propoxy-phenyl]-methylidene]-1-phenyl-pyrazolidine-3,5-dione;
4-[1-[2-((4R,5S)-4-carboxy-2,2-dimethyl-[1,3]dioxolan-5-ylmethoxy)-3-methyl-4-propoxy-phenyl]-methylidene]-1-phenyl-pyrazolidine-3,5-dione;
1-phenyl-4-[1-(5-propoxy-1,2,3,4-tetrahydro-isoquinolin-8-yl)-methylidene]-pyrazolidine-3,5-dione and its formate salt;
4-[1-[2-(2,3-dihydroxy-propoxy)-3-methyl-4-propoxy-phenyl]-methylidene]-1-phenyl-pyrazolidine-3,5-dione;
4-[1-[2-((2S)-2,3-dihydroxy-propoxy)-3-methyl-4-propoxy-phenyl]-methylidene]-1-phenyl-pyrazolidine-3,5-dione;
4-[1-[2-((2R)-2,3-dihydroxy-propoxy)-3-methyl-4-propoxy-phenyl]-methylidene]-1-phenyl-pyrazolidine-3,5-dione;
4-[1-[2-((2S,3R)-2,3-dihydroxy-3-ethoxycarbonyl-propoxy)-3-methyl-4-propoxy-phenyl]-methylidene]-1-phenyl-pyrazolidine-3,5-dione;
4-[1-[2-((2S,3R)-3-carboxy-2,3-dihydroxy-propoxy)-3-methyl-4-propoxy-phenyl]-methylidene]-1-phenyl-pyrazolidine-3,5-dione;
ethyl 4-(4-benzylidene-3,5-dioxo-pyrazolidin-1-yl)-benzoate;
ethyl 4-[4-(2-hydroxy-3-methoxy-benzylidene)-3,5-dioxo-pyrazolidin-1-yl]-benzoate;
ethyl 4-[4-(2-methoxy-benzylidene)-3,5-dioxo-pyrazolidin-1-yl]-benzoate;
ethyl 4-[4-(3-methoxy-benzylidene)-3,5-dioxo-pyrazolidin-1-yl]-benzoate;
ethyl 4-(3,5-dioxo-4-pyridin-3-ylmethylene-pyrazolidin-1-yl)-benzoate;
ethyl 4-(3,5-dioxo-4-thiophen-3-ylmethylene-pyrazolidin-1-yl)-benzoate;
ethyl 4-[4-(2,3-dimethyl-4-propoxy-benzylidene)-3,5-dioxo-pyrazolidin-1-yl]-benzoate;
4-(3-methyl-4-propoxy-benzylidene)-1-pyridin-2-yl-pyrazolidine-3,5-dione;
4-(2,3-dimethyl-4-propoxy-benzylidene)-1-pyridin-2-yl-pyrazolidine-3,5-dione;
1-(4-bromo-phenyl)-4-(2,3-dimethyl-4-propoxy-benzylidene)-pyrazolidine-3,5-dione;
4-(2,3-dimethyl-4-propoxy-benzylidene)-1-(4-methoxy-phenyl)-pyrazolidine-3,5-dione;
4-[4-(2,3-dimethyl-4-propoxy-benzylidene)-3,5-dioxo-pyrazolidin-1-yl]-benzonitrile;
4-(2,3-dimethyl-4-propoxy-benzylidene)-1-(4-fluoro-phenyl)-pyrazolidine-3,5-dione;
4-(2,3-dimethyl-4-propoxy-benzylidene)-1-(4-methyl-phenyl)-pyrazolidine-3,5-dione;
1-(2-chloro-phenyl)-4-(2,3-dimethyl-4-propoxy-benzylidene)-pyrazolidine-3,5-dione;
4-(2,3-dimethyl-4-propoxy-benzylidene)-1-(2-methyl-phenyl)-pyrazolidine-3,5-dione;
4-(2,3-dimethyl-4-propoxy-benzylidene)-pyrazolidine-3,5-dione;
4-(4-cyclopentyloxy-2,3-dimethyl-benzylidene)-pyrazolidine-3,5-dione;
4-(4-propoxy-5,6,7,8-tetrahydro-naphthalen-1-ylmethylene)-pyrazolidine-3,5-dione;
4-(2,3-dimethyl-4-pent-1-ynyl-benzylidene)-pyrazolidine-3,5-dione;
4-(2,3-dimethyl-4-pentyl-benzylidene)-pyrazolidine-3,5-dione;
4-(4-propoxy-naphthalen-1-ylmethylene)-pyrazolidine-3,5-dione;
4-(7-propoxy-indan-4-ylmethylene)-pyrazolidine-3,5-dione;
4-(2-methoxy-3-methyl-4-propoxy-benzylidene)-pyrazolidine-3,5-dione;
4-(3-methyl-2,4-dipropoxy-benzylidene)-pyrazolidine-3,5-dione;
4-(2,3-dimethyl-4-propoxy-benzylidene)-1-methyl-pyrazolidine-3,5-dione;
4-(2,3-dimethyl-4-propoxy-benzylidene)-1-pyridin-4-yl-pyrazolidine-3,5-dione; and
1-acetyl-4-[1-(2,3-dimethyl-4-propoxy-phenyl)-methylidene]-pyrazolidine-3,5-dione.

18. A compound selected from the group consisting of:
a pyrazolidinedione derivative of formula (III′), 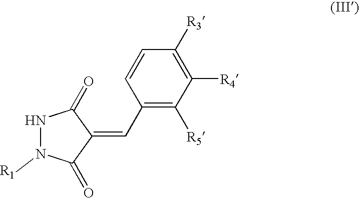
geometric isomers thereof, tautomers thereof, and mixtures thereof; or a salt, ester or prodrug of said compound.
wherein
R₁ is hydrogen, optionally substituted alkyl, cycloalkyl, aryl, arylalkyl, heteroaryl, heteroarylalkyl or alkanoyl;
R₃′ is alkyl, alkenyl, alkynyl, alkoxy, hydroxyalkoxy, alkoxyalkoxy, alkenyloxy, cycloalkoxy, cycloalkylalkoxy or alkylsulfonyloxy;
R₄′ is halogen, hydroxy, alkyl or alkoxy; and
R₅′ is halogen, hydroxy, alkyl, alkoxy, alkoxyalkoxy, hydroxyalkoxy, dihydroxyalkoxy, alkanoyloxyalkoxy, carboxyalkoxy, carboxy-hydroxyalkoxy, carboxy-dihydroxyalkoxy, alkoxycarbonylalkoxy, alkoxycarbonyl-hydroxyalkoxy, alkoxycarbonyl-dihydroxyalkoxy, carbamoylalkoxy, N-alkylcarbamoylalkoxy, N,N-dialkyaminolalkoxy, morpholin-4-ylalkoxy, piperidin-1-ylalkoxy, morpholin-4-ylcarbonylalkoxy, 2,2-dialkyl[1,3]dioxolan-4-ylalkoxy or 2,2-dialkyl-4-carboxy[1,3]dioxolan-5-ylalkoxy; or
R₄′ and R₅′, together with the phenyl ring to which they are attached, form a fused, optionally substituted carbocyclic or heterocyclic ring system;
with the proviso that if R₁ is phenyl and R₃′ is methoxy, R₄′ and R₅′ may not both be methoxy.

19. The compound according to claim 18,
wherein
R₁ is other than alkanoyl;
R₃′ is alkyl, alkenyl, alkynyl, alkoxy, hydroxyalkoxy, alkoxyalkoxy, alkenyloxy, cycloalkoxy or cycloalkylalkoxy; and
R₄′ and R₅′, each independently of the other, are halogen, hydroxy, alkyl or alkoxy; or
R₄′ und R₅′, together with the phenyl ring to which they are attached, form a fused, optionally substituted carbocyclic or heterocyclic ring system.

20. The compound according to claim 18,
wherein
R₃′ is alkyl, alkenyl, alkynyl, alkoxy, cycloalkoxy, cycloalkylalkoxy, hydroxyalkoxy or alkoxyalkoxy,
R₄′ is halogen, alkyl or alkoxy, and
R₅′ is alkyl or alkoxy.

21. The compound according to claim 20,
wherein
R₃′ is methyl, ethyl, propyl, iso-propyl, butyl, iso-butyl, tert-butyl, pentyl, hexyl, but-1-enyl, pent-1-enyl, but-1-ynyl, pent-1-ynyl, methoxy, ethoxy, propoxy, butoxy, iso-butoxy, 3-methyl-butoxy, pentyloxy, cyclopentyloxy, hexyloxy, cyclopropylmethoxy, cyclobutylmethoxy, 2-hydroxy-ethoxy, 2-methoxy-ethoxy,
R₄′ is chloro, bromo, methyl or methoxy, and
R₅′ is methyl or methoxy.

22. The compound according to claim 18,
wherein
R₃′ is alkoxy, and
R₄′ and R₅′ together with the phenyl ring to which they are attached, form an optionally substituted naphthalene, tetrahydronaphthalene, indane, 1H-indene, isoquinoline, dihydro-benzo[1,4]dioxine or benzo[1,3]dioxole ring system.

23. The compound according to claim 22, wherein R₃′ is propoxy, and
R₄′ and R₅′ together with the phenyl ring to which they are attached, form a naphthalene-1-yl, indan-4-yl, isoquinolin-5-yl, isoquinolin-8-yl, 1,2,3,4-tetrahydroisoquinolin-8-yl, 2-alkoxycarbonyl-1,2,3,4-tetrahydroisoquinolin-8-yl or 5,6,7,8-tetrahydronaphthalen-1-yl moiety.

24. The compound according to claim 18,
wherein
R₃′ is methoxy, propoxy, cyclopentyloxy, pent-1-ynyl or ethanesulfonyloxy;
R₄′ is methyl;
R₅′ is hydroxy, methyl, pentyl, methoxy, propoxy, 2-methoxyethoxy, 2-hydroxyethoxy, 3-hydroxypropoxy, 4-hydroxybutoxy, 2,3-dihydroxypropoxy, 4-acetoxybutoxy, carboxymethoxy, 3-carboxypropoxy, 4-carboxybutoxy, 3-carboxy-2-hydroxypropoxy, 3-carboxy-2,3-dihydroxypropoxy, ethoxycarbonylmethoxy, 3-ethoxycarbonylpropoxy, 4-ethoxycarbonylbutoxy, 3-ethoxycarbonyl-2-hydroxypropoxy, 3-ethoxycarbonyl-2,3-dihydroxypropoxy, carbamoylmethoxy 3-N-ethylcarbamoylpropoxy, 4-N-ethylcarbamoylbutoxy, 2-N,N-dimethylaminoethoxy, 3-N,N-dmethylaminopropoxy, 2-(morpholin-4-yl)-ethyoxy, 2-(piperidin-1-yl)-ethoxy, 3-(morpholin-4-yl)-carbonylpropoxy, 4-(morpholin-4-yl)-carbonylbutoxy, 2,2-dimethyl[1,3]dioxolan-4-ylmethoxy or 2,2-dimethyl-4-carboxy[1,3]dioxolan-5-ylmethoxy; or
R₄′ and R₅′, together with the phenyl ring to which they are attached, form a naphthalen-1-yl, 5,6,7,8-tetrahydronaphthalen-1-yl, indan-4-yl, 1,2,3,4,-tetraisoquinolin-8-yl or 2-tert-butoxycarbonyl-1,2,3,4,-tetraisoquinolin-8-yl moiety.

25. A compound selected from
4-[1-[2-((2S)-3-carboxy-2-hydroxy-propoxy)-3-methyl-4-propoxy-phenyl]-methylidene]-1-phenyl-pyrazolidine-3,5-dione;
4-[1-[2-((2R)-3-carboxy-2-hydroxy-propoxy)-3-methyl-4-propoxy-phenyl]-methylidene]-1-phenyl-pyrazolidine-3,5-dione;
4-[1-[2-(3-carboxy-propoxy)-3-methyl-4-propoxy-phenyl]-methylidene]-1-phenyl-pyrazolidine-3,5-dione;
4-[1-[2-(4-carboxy-butyloxy)-3-methyl-4-propoxy-phenyl]-methylidene]-1-phenyl-pyrazolidine-3,5-dione;
4-[1-[2-(2,3-dihydroxy-propoxy)-3-methyl-4-propoxy-phenyl]-methylidene]-1-phenyl-pyrazolidine-3,5-dione;
4-[1-[2-((2S)-2,3-dihydroxy-propoxy)-3-methyl-4-propoxy-phenyl]-methylidene]-1-phenyl-pyrazolidine-3,5-dione;
4-[1-[2-((2R)-2,3-dihydroxy-propoxy)-3-methyl-4-propoxy-phenyl]-methylidene]-1-phenyl-pyrazolidine-3,5-dione;
4-[1-[2-((2S,3R)-3-carboxy-2,3-dihydroxy-propoxy)-3-methyl-4-propoxy-phenyl]-methylidene]-1-phenyl-pyrazolidine-3,5-dione;
4-[1-[2-(5-ethylamino-5-oxo-pentyloxy)-3-methyl-4-propoxy-phenyl]-methylidene]-1-phenyl-pyrazolidine-3,5-dione;
4-[1-[2-(3-hydroxy-propoxy)-3-methyl-4-propoxy-phenyl]-methylidene]-1-phenyl-pyrazolidine-3,5-dione;
4-[1-[2-(4-hydroxy-butoxy)-3-methyl-4-propoxy-phenyl]-methylidene]-1-phenyl-pyrazolidine-3,5-dione;
4-[1-[2-(2-hydroxy-ethoxy)-3-methyl-4-propoxy-phenyl]-methylidene]-1-phenyl-pyrazolidine-3,5-dione;
4-[1-[2-((2S,3R)-2,3-dihydroxy-3-ethoxycarbonyl-propoxy)-3-methyl-4-propoxy-phenyl]-methylidene]-1-phenyl-pyrazolidine-3,5-dione;
4-[1-[2-(ethoxycarbonyl-methoxy)-3-methyl-4-propoxy-phenyl]-methylidene]-1-phenyl-pyrazolidine-3,5-dione; and
4-[1-[3-methyl-2-(4-morpholin-4-yl-4-oxo-butoxy)-4-propoxy-phenyl]-methylidene]-1-phenyl-pyrazolidine-3,5-dione.

26. A compound selected from
1-phenyl-4-[1-(5-propoxy-isoquinolin-8-yl)-methylidene]-pyrazolidine-3,5-dione;
4-[1-[2-(4-ethylamino-4-oxo-butoxy)-3-methyl-4-propoxy-phenyl]-methylidene]-1-phenyl-pyrazolidine-3,5-dione;
4-[1-[2-(4-ethoxycarbonyl-butoxy)-3-methyl-4-propoxy-phenyl]-methylidene]-1-phenyl-pyrazolidine-3,5-dione;
4-[1-[2-((2R)-3-ethoxycarbonyl-2-hydroxy-propoxy)-3-methyl-4-propoxy-phenyl]-methylidene]-1-phenyl-pyrazolidine-3,5-dione;
4-[1-[2-(4-acetoxy-butoxy)-3-methyl-4-propoxy-phenyl]-methylidene]-1-phenyl-pyrazolidine-3,5-dione;
4-[1-[2-(3-ethoxycarbonyl-propoxy)-3-methyl-4-propoxy-phenyl]-methylidene]-1-phenyl-pyrazolidine-3,5-dione;
4-[1-[2-(2-methoxy-ethoxy)-3-methyl-4-propoxy-phenyl]-methylidene]-1-phenyl-pyrazolidine-3,5-dione;
4-[1-[3-methyl-2-(5-morpholin-4-yl-5-oxo-pentyloxy)-4-propoxy-phenyl]-methylidene]-1-phenyl-pyrazolidine-3,5-dione;
1-phenyl-4-[1-(8-propoxy-isoquinolin-5-yl)-methylidene]-pyrazolidine-3,5-dione;
4-[1-[2-(2-amino-2-oxo-ethyloxy)-3-methyl-4-propoxy-phenyl]-methylidene]-1-phenyl-pyrazolidine-3,5-dione;
4-[1-[3-methyl-2-(2-morpholin-4-yl-ethoxy)-4-propoxy-phenyl]-methylidene]-1-phenyl-pyrazolidine-3,5-dione and its hydrochloride salt;
4-[1-[2-(2,2-dimethyl-[1,3]dioxolan-4-ylmethoxy)-3-methyl-4-propoxy-phenyl]-methylidene]-1-phenyl-pyrazolidine-3,5-dione;
4-(2,3-dimethyl-4-propoxy-benzylidene)-1-phenyl-pyrazolidine-3,5-dione;
4-[1-[2-((4R,5S)-4-carboxy-2,2-dimethyl-[1,3]dioxolan-5-ylmethoxy)-3-methyl-4-propoxy-phenyl]-methylidene]-1-phenyl-pyrazolidine-3,5-dione;
4-[1-[2-((4R)-2,2-dimethyl-[1,3]dioxolan-4-ylmethoxy)-3-methyl-4-propoxy-phenyl]-methylidene]-1-phenyl-pyrazolidine-3,5-dione;
4-[1-[2-(3-dimethylamino-propoxy)-3-methyl-4-propoxy-phenyl]-methylidene]-1-phenyl-pyrazolidine-3,5-dione and its hydrochloride salt;
1-phenyl-4-[1-(7-propoxy-indan-4-yl)-methylidene]-pyrazolidine-3,5-dione;
1-phenyl-4-(4-propoxy-5,6,7,8-tetrahydro-naphthalen-1-ylmethylene)-pyrazolidine-3,5-dione;
1-phenyl-4-[1-(5-propoxy-1,2,3,4-tetrahydro-isoquinolin-8-yl)-methylidene]-pyrazolidine-3,5-dione and its formate salt;
4-[1-[2-(2-dimethylamino-ethoxy)-3-methyl-4-propoxy-phenyl]-methylidene]-1-phenyl-pyrazolidine-3,5-dione and its hydrochloride salt;
4-[1-[2-(carboxy-methoxy)-3-methyl-4-propoxy-phenyl]-methylidene]-1-phenyl-pyrazolidine-3,5-dione;
4-[1-[2-((4S)-2,2-dimethyl-[1,3]dioxolan-4-ylmethoxy)-3-methyl-4-propoxy-phenyl]-methylidene]-1-phenyl-pyrazolidine-3,5-dione;
4-(3-methyl-2,4-dipropoxy-benzylidene)-pyrazolidine-3,5-dione;
4-[1-(3-methyl-2,4-dipropoxy-phenyl)-methylidene]-1-phenyl-pyrazolidine-3,5-dione;
4-(4-propoxy-5,6,7,8-tetrahydro-naphthalen-1-ylmethylene)-pyrazolidine-3,5-dione;
4-[1-(2-methoxy-3-methyl-4-propoxy-phenyl)-methylidene]-1-phenyl-pyrazolidine-3,5-dione;
4-(2-methoxy-3-methyl-4-propoxy-benzylidene)-pyrazolidine-3,5-dione;
4-(7-propoxy-indan-4-ylmethylene)-pyrazolidine-3,5-dione;
4-[1-(2-tert-butoxycarbonyl-5-propoxy-1,2,3,4-tetrahydro-isoquinolin-8-yl)-methylidene]-1-phenyl-pyrazolidine-3,5-dione; and
4-(2,3-dimethyl-4-propoxy-benzylidene)-1-(4-fluoro-phenyl)-pyrazolidine-3,5-dione.

27. A compound selected from
1-phenyl-4-(4-propoxy-naphthalen-1-ylmethylene)-pyrazolidine-3,5-dione;
4-[1-[3-methyl-2-(2-piperidin-1-yl-ethoxy)-4-propoxy-phenyl]-methylidene]-1-phenyl-pyrazolidine-3,5-dione and its hydrochloride salt;
4-(2,4-dimethoxy-3-methyl-benzylidene)-1-phenyl-pyrazolidine-3,5-dione;
4-(2,3-dimethyl-4-ethanesulfonyloxy-benzylidene)-1-phenyl-pyrazolidine-3,5-dione;
4-(2,3-dimethyl-4-propoxy-benzylidene)-1-pyridin-2-yl-pyrazolidine-3,5-dione;
4-(2,3-dimethyl-4-propoxy-benzylidene)-pyrazolidine-3,5-dione;
4-(2,3-dimethyl-4-methoxybenzylidene)-1-phenyl-pyrazolidine-3,5-dione;
4-(2,3-dimethyl-4-pent-1-ynyl-benzylidene)-1-phenyl-pyrazolidine-3,5-dione;
4-(4-propoxy-naphthalen-1-ylmethylene)-pyrazolidine-3,5-dione;
4-[4-(2,3-dimethyl-4-propoxy-benzylidene)-3,5-dioxo-pyrazolidin-1-yl]-benzonitrile;
4-[1-(2-hydroxy-3-methyl-4-propoxy-phenyl)-methylidene]-1-phenyl-pyrazolidine-3,5-dione;
4-(2,3-dimethyl-4-pentyl-benzylidene)-1-phenyl-pyrazolidine-3,5-dione;
4-(2,3-dimethyl-4-pentyl-benzylidene)-pyrazolidine-3,5-dione;
1-(2-chloro-phenyl)-4-(2,3-dimethyl-4-propoxy-benzylidene)-pyrazolidine-3,5-dione;
4-(2,3-dimethyl-4-pent-1-ynyl-benzylidene)-pyrazolidine-3,5-dione;
4-(2,3-dimethyl-4-propoxy-benzylidene)-1-pyridin-4-yl-pyrazolidine-3,5-dione;
4-(2,3-dimethyl-4-propoxy-benzylidene)-1-(4-methyl-phenyl)-pyrazolidine-3,5-dione;
4-(4-cyclopentyloxy-2,3-dimethyl-benzylidene)-pyrazolidine-3,5-dione;
4-(4-cyclopentyloxy-2,3-dimethyl-benzylidene)-1-phenyl-pyrazolidine-3,5-dione;
4-(2,3-dimethyl-4-propoxy-benzylidene)-1-(4-methoxy-phenyl)-pyrazolidine-3,5-dione;
1-(4-bromo-phenyl)-4-(2,3-dimethyl-4-propoxy-benzylidene)-pyrazolidine-3,5-dione;
4-(2,3-dimethyl-4-propoxy-benzylidene)-1-(2-methyl-phenyl)-pyrazolidine-3,5-dione;
4-(2,3-dimethyl-4-propoxy-benzylidene)-1-methyl-pyrazolidine-3,5-dione;
ethyl 4-[4-(2,3-dimethyl-4-propoxy-benzylidene)-3,5-dioxo-pyrazolidin-1-yl]-benzoate; and
1-acetyl-4-[1-(2,3-dimethyl-4-propoxy-phenyl)-methylidene]-pyrazolidine-3,5-dione.

28. (canceled)

29. A pharmaceutical composition comprising the compound according to claim 18 and a pharmaceutically inert carrier.

30. A method for preventing or treating a disease or a condition associated with platelet aggregation, said method comprising administering to a mammal in need thereof the compound according to claim 18.

31. A process for the manufacture of the compound according to claim 18, comprising condensing a pyrazolidinedione of formula (IV), as shown in the following reaction scheme wherein R₁, R_{3′}, R_{4′} and R_{5′} are as defined in claim 18, 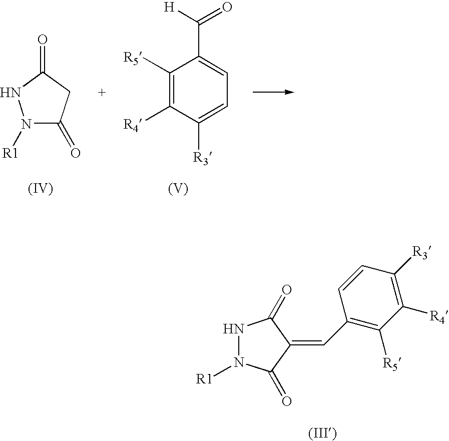
with an aldehyde of the above general formula (V), any reactive group which may be present in the compounds of formulae IV and V being appropriately protected, and, if necessary, splitting off from the condensation product any protecting group(s).

32. The process of claim 31, wherein the pyrazolidinedione of the general formula (IV) is selected from the group consisting of:
1-pyridin-2-yl-pyrazolidine-3,5-dione;
4-(3,5-dioxo-pyrazolidin-1-yl)-benzonitrile; and
1-pyridin-4-yl-pyrazolidine-3,5-dione.

33. The process of claim 31, wherein the aldehyde of the general formula (V) is selected from the group consisting of:
4-cyclopentyloxy-2,3-dimethyl-benzaldehyde;
4-propoxy-5,6,7,8-tetrahydro-naphthalene-1-carbaldehyde;
2,3-dimethyl-4-pent-1-ynyl-benzaldehyde;
2,3-dimethyl-4-pentyl-benzaldehyde;
7-propoxy-indan-4-carbaldehyde;
5-propoxy-isoquinoline-8-carbaldehyde;
8-propoxy-isoquinoline-5-carbaldehyde;
2-tert-butyloxycarbonyl-8-formyl-5-propoxy-1,2,3,4-tetrahydroisoquinoline;
2,3-dimethyl-4-ethanesulfonyloxybenzaldehyde;
2-hydroxy-3-methyl-4-propoxy-benzaldehyde;
2-methoxy-3-methyl-4-propoxy-benzaldehyde;
2-(2-methoxy-ethoxy)-3-methyl-4-propoxy-benzaldehyde;
2-(2-hydroxy-ethoxy)-3-methyl-4-propoxy-benzaldehyde;
2-(3-hydroxy-propoxy)-3-methyl-4-propoxy-benzaldehyde;
2-(4-acetoxy-butoxy)-3-methyl-4-propoxy-benzaldehyde;
2-(4-hydroxy-butoxy)-3-methyl-4-propoxy-benzaldehyde;
ethyl (6-formyl-2-methyl-3-propoxy-phenoxy)-acetate;
(6-formyl-2-methyl-3-propoxy-phenoxy)-acetic acid;
2-(6-formyl-2-methyl-3-propoxy-phenoxy)-acetamide;
ethyl 4-(6-formyl-2-methyl-3-propoxy-phenoxy)-butanoate;
4-(6-formyl-2-methyl-3-propoxy-phenoxy)-butanoic acid;
4-(6-formyl-2-methyl-3-propoxy-phenoxy)-butanoic acid ethylamide;
3-methyl-2-(4-morpholin-4-yl-4-oxo-butoxy)-4-propoxy-benzaldehyde;
ethyl 5-(6-formyl-2-methyl-3-propoxy-phenoxy)-pentanoate;
5-(6-formyl-2-methyl-3-propoxy-phenoxy)-pentanoic acid;
5-(6-formyl-2-methyl-3-propoxy-phenoxy)-pentanoic acid ethylamide;
3-methyl-2-(5-morpholin-4-yl-5-oxo-pentyloxy)-4-propoxy-benzaldehyde;
2-(2-dimethylamino-ethoxy)-3-methyl-4-propoxy-benzaldehyde and its hydrochloride salt;
3-methyl-2-(2-morpholin-4-yl-ethoxy)-4-propoxy-benzaldehyde and its hydrochloride salt;
3-methyl-2-(2-piperidin-1-yl-ethoxy)-4-propoxy-benzaldehyde and its hydrochloride salt;
2-(3-dimethylamino-propoxy)-3-methyl-4-propoxy-benzaldehyde and its hydrochloride salt;
2-(2,2-dimethyl-[1,3]dioxolan-4-ylmethoxy)-3-methyl-4-propoxy-benzaldehyde;
2-((4R)-2,2-dimethyl-[1,3]dioxolan-4-ylmethoxy)-3-methyl-4-propoxy-benzaldehyde;
2-((4S)-2,2-dimethyl-[1,3]dioxolan-4-ylmethoxy)-3-methyl-4-propoxy-benzaldehyde;
ethyl (3R)-4-(6-formyl-2-methyl-3-propoxy-phenoxy)-3-hydroxy-butanoate;
(3R)-4-(6-formyl-2-methyl-3-propoxy-phenoxy)-3-hydroxy-butanoic acid;
ethyl (3S)-4-(6-formyl-2-methyl-3-propoxy-phenoxy)-3-hydroxy-butanoate;
(3S)-4-(6-formyl-2-methyl-3-propoxy-phenoxy)-3-hydroxy-butanoic acid;
methyl (4R,5S)-5-(6-formyl-2-methyl-3-propoxy-phenoxymethyl)-2,2-dimethyl-[1,3]dioxolane-4-carboxylate;
(4R,5S)-5-(6-formyl-2-methyl-3-propoxy-phenoxymethyl)-2,2-dimethyl-[1,3]dioxolane-4-carboxylic acid.
